SEQUENCE LISTING (26 sequences)

SEQ ID NO: 1 - protein, 5 aa, Artificial Sequence
  Description of Artificial Sequence Synthetic peptide
VPGXG

SEQ ID NO: 2 - protein, 10 aa, Artificial Sequence
  Description of Artificial Sequence Synthetic peptide
VPGXGVPGXG

SEQ ID NO: 3 - protein, 350 aa, Artificial Sequence
  Description of Artificial Sequence Synthetic polypeptide
XVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGXXVPGXGX

SEQ ID NO: 4 - protein, 10 aa, Artificial Sequence
  Description of Artificial Sequence Synthetic peptide
VPGFGVPGFG

SEQ ID NO: 5 - protein, 10 aa, Artificial Sequence
  Description of Artificial Sequence Synthetic peptide
VPGIGVPGLG

SEQ ID NO: 6 - protein, 10 aa, Artificial Sequence
  Description of Artificial Sequence Synthetic peptide
VPGYGVPGFG

SEQ ID NO: 7 - protein, 10 aa, Artificial Sequence
  Description of Artificial Sequence Synthetic peptide
VPGFGVPGYG

SEQ ID NO: 8 - protein, 10 aa, Artificial Sequence
  Description of Artificial Sequence Synthetic peptide
VPGWGVPGFG

SEQ ID NO: 9 - protein, 10 aa, Artificial Sequence
  Description of Artificial Sequence Synthetic peptide
VPGFGVPGWG

SEQ ID NO: 10 - protein, 10 aa, Artificial Sequence
  Description of Artificial Sequence Synthetic peptide
VPGYGVPGYG

SEQ ID NO: 11 - protein, 10 aa, Artificial Sequence
  Description of Artificial Sequence Synthetic peptide
VPGWGVPGWG

SEQ ID NO: 12 - protein, 5 aa, Artificial Sequence
  Description of Artificial Sequence Synthetic peptide
VPGFG

SEQ ID NO: 13 - protein, 5 aa, Artificial Sequence
  Description of Artificial Sequence Synthetic peptide
VPGYG

SEQ ID NO: 14 - protein, 5 aa, Artificial Sequence
  Description of Artificial Sequence Synthetic peptide
VPGWG

SEQ ID NO: 15 - protein, 5 aa, Artificial Sequence
  Description of Artificial Sequence Synthetic peptide
VPAYG

SEQ ID NO: 16 - protein, 5 aa, Artificial Sequence
  Description of Artificial Sequence Synthetic peptide
APGYG

SEQ ID NO: 17 - protein, 5 aa, Artificial Sequence
  Description of Artificial Sequence Synthetic peptide
IPGYG

SEQ ID NO: 18 - protein, 5 aa, Artificial Sequence
  Description of Artificial Sequence Synthetic peptide
LPGYG

SEQ ID NO: 19 - protein, 5 aa, Artificial Sequence
  Description of Artificial Sequence Synthetic peptide
VPGVG

SEQ ID NO: 20 - protein, 10 aa, Artificial Sequence
  Description of Artificial Sequence Synthetic peptide
VPGVGVPGKG

SEQ ID NO: 21 - protein, 5 aa, Artificial Sequence
  Description of Artificial Sequence Synthetic peptide
VPGLG

SEQ ID NO: 22 - protein, 5 aa, Artificial Sequence
  Description of Artificial Sequence Synthetic peptide
VPGIG

SEQ ID NO: 23 - protein, 5 aa, Artificial Sequence
  Description of Artificial Sequence Synthetic peptide
VPGXG

SEQ ID NO: 24 - protein, 10 aa, Artificial Sequence
  Description of Artificial Sequence Synthetic peptide
VPGXGVPGXG

SEQ ID NO: 25 - protein, 776 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 26 - protein, 250 aa, Artificial Sequence
  Description of Artificial Sequence Synthetic polypeptide
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

(19) United States
(12) Patent Application Publication
(10) Pub. No.: US 2015/0218252 A1
(43) Pub. Date: Aug. 6, 2015

(54) SELF-ASSEMBLING PEPTIDES, PEPTIDE NANOSTRUCTURES AND USES THEREOF

(71) Applicant: President and Fellows of Harvard College, Cambridge, MA (US)

(72) Inventors: Donald E. Ingber, Boston, MA (US); Kenny Roberts, Brookline, MA (US)

(21) Appl. No.: 14/409,839

(22) PCT Filed: Jun. 20, 2013
(86) PCT No.: PCT/US2013/046821

§ 371 Date: Dec. 19, 2014

Related U.S. Application Data
(60) Provisional application No. 61/662,007, filed on Jun. 20, 2012.

(51) Int. Cl. C07K 14/78; A61K 9/16; A61K 47/48

(52) U.S. Cl. CPC C07K 14/78; A61K 47/48246; A61K 9/1658

(57) ABSTRACT

Provided herein relates to self-assembling peptides and various nanostructures self-assembled from the isolated peptides. In some embodiments, the self-assembling peptides can form a nanostructure, e.g., a nanoparticle or microparticle, for use in various biomedical applications such as drug delivery or tissue engineering. In some embodiments, the nanostructures can comprise an agent, e.g., a biological molecule. The agent can be encapsulated or entrapped in the nanostructures during formation of the nanostructures. Alternatively or additionally, the agent can be integrated directly or indirectly (e.g., via a linker or a conjugation or crosslinking agent) to the self-assembling peptide structure, prior to formation of the nanostructures. In some embodiments where the agent is a peptide-based agent, unitary peptide nano structures, rather than nanoparticles that are formed and later covalently modified, can be generated.

### CROSS REFERENCE TO RELATED APPLICATIONS

This application claims the benefit under 35 U.S.C. §119(e) of U.S. Provisional Application No. 61/662,007 filed Jun. 20, 2012, the content of which is incorporated herein by reference in its entirety.

### GOVERNMENT SUPPORT

This invention was made with government support under grant no. BC074986 awarded by Department of Defense. The government has certain rights in the invention.

### TECHNICAL FIELD

The present invention relates to isolated self-assembling peptides, nanostructures self-assembled from the isolated peptides, and fabrication methods and applications thereof.

### BACKGROUND

Stimuli-responsive polymers are “smart” materials that can adapt to surrounding environments, regulate transport of ions and molecules, change wettability and adhesion of different species on external stimuli, or convert chemical and biochemical signals into optical, electrical, thermal and mechanical signals, and vice versa. Thus, stimuli-responsive polymers can be used for various biomedical applications including drug delivery, tissue engineering, and biosensors as well as non-medical applications such as microelectromechanical systems, coatings and textiles.

Stimuli-responsive nanostructures or nanomaterials (e.g., nanoparticles or microparticles) composed of peptides are desirable for various biomedical applications including drug delivery and tissue engineering as they can degrade into single amino acids. In addition, unlike other nanostructure materials, e.g., polymer, products of peptide synthesis can be purified to up to 98%, avoiding molecular polydispersity and thus issues with the reproducibility of physicochemical properties. Further, properties of peptide structure can be readily modulated, e.g., by introduction of amino acid point mutations. Accordingly, there is still a strong need for engineering a biodegradable stimuli-responsive nanostructure or nanomaterial, which can be synthesized and purified in a simple process.

### SUMMARY

Various aspects provided herein relate to isolated short peptides, and peptide nanostructures that are self-assembled from the short peptides, as well as articles, compositions, and kits comprising the short peptides and/or self-assembled peptide nanostructures. Methods of forming the peptide nanostructures and using the short peptides and/or peptide nanostructures for various applications are also provided herein. In some embodiments, the short peptides and/or self-assembled nanostructures are stimuli-responsive, e.g., pH-responsive and/or temperature-responsive, and can thus be adapted for various applications such as drug delivery, biotechnology, bioengineering and/or tissue engineering. The inventors have discovered that, in some embodiments, short peptides (e.g., as short as 5 amino acid residues in length such as about 5-10 amino acid residues in length) can spontaneously self-assemble in aqueous media to form discrete spherical particles, for example, with a size in a range of about 50 nm to about 2 μm. The spherical particles can be polydisperse or monodisperse. In some embodiments, the stability of the peptide nanostructures can be tunable—for example, from hours to days to weeks to months—without the need for excipients, stabilizers, and/or crosslinkers. In addition, the inventors have demonstrated that, in some embodiments, the short peptides can be modified, for example, for conjugation to an agent or a substrate, such as a polymer, a ligand, a protein, or a nanoparticle. In some embodiments where the agent is a peptide-based agent, unitary peptide nanostructures, rather than nanoparticles that are formed and later covalently modified, can be generated. The inventors have also demonstrated the versatility of the short peptides to form different sizes and/or shapes of nanostructures, including but not limited to nanospheres, nanovesicles, nanorods, nanotubes, and nanofibers, based on different formulation and/or processing conditions.

Accordingly, one aspect provided herein is directed to an isolated peptide consisting essentially of an amino acid sequence of (Y₁-X₁-X₂-X₃-X₄-X₃-Y₂)ₙ conjugated to an entity, wherein X₁ is valine (Val) or a conservative substitution thereof; X₂ is proline (Pro) or a conservative substitution thereof; X₃ is glycine (Gly) or a conservative substitution thereof; X₄ in each n^{th} unit is independently an amino acid residue; and n is an integer from 1 to 50.

In some embodiments, n can be an integer from 1 to 25. In other embodiments, n can be an integer from 1 to 10. In other embodiments, n can be an integer from 1 to 2. In one embodiment, n is an integer of 1. In another embodiment, n is an integer of 2.

In some embodiments, when n is 4, at least one X₄ is not valine. In other embodiments, when n is 1, X₄ is not valine.

In embodiments of the isolated peptide described herein, Y₁ and Y₂ are each independently a linker. Exemplary linker can include, but is not limited to, a chemical linker (e.g., a bond), a peptidyl linker (e.g., one amino acid residue or a group of amino acid residues), and a combination thereof. In some embodiments, the sum of Y₁ and Y₂ has no more than 4 amino acid residues. In some embodiments, the combined amino acid sequence of Y₁ and Y₂ does not include a sequence or repeating units of (VPGX₄G).

The entity conjugated to the amino acid sequence of the isolated peptide can include, without limitations, —H, —OH, a chemical functional group, a ligand, a therapeutic agent, a binding molecule, a coupling molecule, a peptide-modifying molecule, a substrate, and any combinations thereof. In some embodiments, when the entity is a substrate and the amino acid sequence is VPGX₄G or (VPGX₄G)₂, the substrate is not a biodegradable non-amino acid moiety, e.g., a biodegradable non-protein polymer selected from the group consisting of monomers or homopolymers of hydroxy acids such as lactide, glycolide, valerolactone, hydroxybutyrate, caprolactone, hydroxyl fatty acids, poly(lactide); poly(glycolide); poly(caprolactone); poly(valerolactone); poly(hydroxybutyrate); poly(lactide-co-glycolide); poly(lactide-co-caprolactone); poly(lactide-co-valerolactone); poly(glycolide-co-caprolactone); poly(glycolide-co-valerolactone); poly(lactide-co-glycolide-co-caprolactone); poly(lactide-co-glycolide-co-valerolactone); and any mixtures thereof.

Any chemical functional group can be conjugated to the amino acid sequence of the isolated peptide. Non-limiting examples of such chemical function groups can include alkyne, halogens, alcohol, ketone, aldehyde, acyl halide, carbonate, carboxylate, carboxylic acid, ester, hydroperoxide, peroxide, ether, hemiacetal, hemiketal, acetal, ketal, acetal, orthoester, amide, amines, imine (e.g., but not limited to primary ketamine, secondary ketamine, primary aldimine, secondary aldimine, ethanimine, and any combinations thereof), imide, azide, azo compound, cyanates, nitrate, nitrile, nitrite, nitro compound, nitroso compound, pyridine and pyridine derivative, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, borinic ester, and any combinations thereof.

The peptide-modifying molecule includes a polypeptide sequence comprising amino acids Pro, Ala, and Ser; a hydroxyethyl starch (HES) derivative; and a combination thereof.

In some embodiments, the amino acid sequence can be (Y₁-Val-Pro-Gly-X₄-Gly-Y₂)ₙ. In some embodiments where Y₁ and Y₂ are each independently one amino acid residue or a group of amino acid residues, the amino acid residue can include at least one non-proteinogenic or non-standard amino acid. In some embodiments, each amino acid residue in the amino acid sequence can be independently D-amino acid or L-amino acid.

When n is 2 or larger, X₄'s in the amino acid sequence can each be the same, or independently different. In some embodiments, at least one X₄ in the amino acid sequence can be different. For example, a first X₄ in the amino acid sequence can be different from a second X₄ within the same sequence.

Generally, X₄ can be any art-recognized amino acid residue, e.g., a hydrophobic amino acid, a hydrophilic amino acid, a non-standard amino acid, or a non-standard amino acid, or a derivative thereof. In some embodiments, at least one X₄ can be a hydrophobic amino acid. In some embodiments, at least two X₄'s can be hydrophobic amino acids. Examples of amino acid residues for X₄ can include, without limitations, phenylalanine (Phe), isoleucine (Ile), leucine (Leu), tyrosine (Tyr), tryptophan (Trp), valine (Val), lysine (Lys), histidine (His), methionine (Met), and a non-standard amino acid and a side-chain modified amino acid.

In some embodiments, the amino acid sequence of the isolated peptide described herein can be 10-amino acid long. Exemplary 10-amino acid sequence of the isolated peptide can include, but are not limited to,

| | |
|---|---|
| (i) | Val-Pro-Gly-Phe-Gly-Val-Pro-Gly-Phe-Gly; |
| (ii) | Val-Pro-Gly-Ile-Gly-Val-Pro-Gly-Leu-Gly; |
| (iii) | Val-Pro-Gly-Tyr-Gly-Val-Pro-Gly-Phe-Gly; |
| (iv) | Val-Pro-Gly-Phe-Gly-Val-Pro-Gly-Tyr-Gly; |
| (v) | Val-Pro-Gly-Trp-Gly-Val-Pro-Gly-Phe-Gly; |
| (vi) | Val-Pro-Gly-Phe-Gly-Val-Pro-Gly-Trp-Gly; |
| (vii) | Val-Pro-Gly-Tyr-Gly-Val-Pro-Gly-Tyr-Gly; |
| and | |
| (viii) | Val-Pro-Gly-Trp-Gly-Val-Pro-Gly-Trp-Gly. |

In some embodiments, the amino acid sequence of the isolated peptide described herein can be 5-amino acid long. Exemplary 5-amino acid sequence of the isolated peptide can include, but are not limited to,

| | |
|---|---|
| (ix) | Val-Pro-Gly-Phe-Gly; |
| (x) | Val-Pro-Gly-Tyr-Gly; |
| (xi) | Val-Pro-Gly-Trp-Gly; |
| (xii) | Val-Pro-Ala-Tyr-Gly; |
| (xiii) | Ala-Pro-Gly-Tyr-Gly; |
| (xiv) | Ile-Pro-Gly-Tyr-Gly; |
| and | |
| (xv) | Leu-Pro-Gly-Tyr-Gly. |

In some embodiments, the amino acid sequence can be conjugated to a ligand. Non-limiting examples of a ligand can include a cellular receptor ligand, a targeting ligand, an antibody or a portion thereof, an antibody-like molecule, an enzyme, an antigen, a small molecule, a protein, a peptide, a peptidomimetic, a carbohydrate, an aptamer, a cytokine, a lectin, a lipid, a plasma albumin, and any combinations thereof.

In some embodiments, the amino acid sequence can be conjugated to a binding molecule, e.g., but not limited to, biotin or avidin.

In some embodiments, the amino acid sequence can be conjugated to a substrate. Exemplary substrate can include, but are not limited to, a gold particle, a silver particle, a magnetic particle, a quantum dot, a fullerene, a carbon tube, a nanowire, a nanofibril, a graphene, and any combinations thereof. In some embodiments, the substrate can include biodegradable protein such as collagen, albumin, silk and any combination thereof.

Another aspect described herein relates to self-assembled peptide nanostructures comprising a plurality of the isolated peptides described herein. The peptide nanostructures can be present in any form or shape, including but not limited to, a particle, a fiber, a rod, a gel, or any combinations thereof. The peptide nanostructures are sensitive or responsive to at least one stimulus, e.g., pH and/or temperature. The response of the peptide nanostructure to the stimulus can be reversible or irreversible. In some embodiments, the response of the peptide nanostructure to the stimulus is reversible.

In some embodiments, the peptide nanostructures can further comprise a biopolymer. The biopolymer can be conjugated to the peptide nanostructures or be blended with a plurality of the isolated peptides during self-assembly.

In some embodiments, the peptide nanostructures can further comprise an active agent. The active agent can be conjugated to or coated on the peptide nanostructures or encapsulated within the peptide nanostructures.

The isolated peptides and/or self-assembled peptide nanostructures can be used in various applications. Accordingly, articles comprising at least one isolated peptide and/or self-assembled peptide nanostructure are also provided herein. Exemplary articles provided herein include, but are not limited to, a tissue engineered scaffold, a medication (e.g., but not limited to, a therapeutic agent, and a preventative agent), a diagnostic agent (including, e.g., but not limited to, an imaging agent), a coating of a medical device, a delivery device or vehicle, a fabric, and any combinations thereof.

In some embodiments, a plurality of the isolated peptides and/or self-assembled peptide nanostructures can be provided in a kit, which further comprises at least one reagent. The reagent can include a coupling agent for linking an isolated peptide and/or peptide nanostructure to a substrate as described herein. In some embodiments, the kit can further comprise an active agent.

Methods and/or applications of using the isolated peptide and/or self-assembled peptide nanostructures are also provided herein. For example, uses of the isolated peptides and/or self-assembled peptide nanostructures described herein to modulate release of an active agent from a composition or an article, to modulate the mechanical stiffness of a matrix, and to induce gel formation of a protein or polymer are described herein.

### BRIEF DESCRIPTION OF THE DRAWINGS

FIG. 1 shows amino acid sequences and corresponding molecular weights of exemplary self-assembling peptide constructs described herein. Based on the amino acid residue(s) of X₄ in the sequence, each indicated amino acid sequence is designated with a name to which is referred throughout the specification.

FIG. 2 shows a protein-coding sequence of human tropoelastin.

FIGS. 3A-3C show characterization data of nano structures formed by self-assembly of one or more embodiments of the self-assembling peptides described herein (with corresponding amino acid sequences shown in FIG. 1) in cold deionized water. FIG. 3A is a SEM image of FF nanoparticles formed from FF peptides prepared at about 80 mg/mL in deionized water and adsorbed on conductive carbon adhesive. The inset shows the FF nanoparticles at a higher magnification. FIG. 3B is a bar graph based on dynamic light scattering (DLS) studies showing the size distribution of the FF nanoparticles with an average hydrodynamic diameter of 765 nm and a polydispersity index (PDI) of 0.27. FIG. 3C is a bar graph based on dynamic light scattering (DLS) studies showing the size distribution of the YF nanoparticles with an average hydrodynamic diameter of 900 nm and a polydispersity index (PDI) of 0.33.

FIGS. 4A-4E show characterization data of nanostructures formed by self-assembly of one or more embodiments of the self-assembling peptides described herein (with corresponding amino acid sequences shown in FIG. 1) in cold saline water. FIG. 4A is a bar graph based on dynamic light scattering (DLS) studies showing the size distribution of the FF nanoparticles with an average hydrodynamic diameter of 191 nm and a polydispersity index (PDI) of 0.18. The FF nanoparticles were formed from FF peptides at a concentration of about 50 mg/mL in the cold saline water. FIG. 4B is a size distribution graph showing effects of the FF peptide concentrations (˜2.5 mg/mL to ˜50 mg/mL) on the resulting nanoparticles. The FF peptides were self-assembled in cold saline (e.g., ˜2° C.-˜4° C.) and the DLS analysis was performed at ˜25° C. FIG. 4C is a bar graph based on dynamic light scattering (DLS) studies showing the size distribution of YF nanoparticles with an average hydrodynamic diameter of 600 nm and a polydispersity index (PDI) of 0.07. The YF nanoparticles were formed from YF peptides at a concentration of about 5 mg/mL in the cold saline water. FIG. 4D is a size distribution graph showing that particle size varies with self-assembly conditions. The blue line corresponds to nanoparticles with an average hydrodynamic diameter of about 765 nm formed by spontaneous self-assembly of the FF peptides in water at room temperature while the red line corresponds to particles (with an average hydrodynamic diameter of about 191 nm) formed by precipitation in cold saline solution. FIG. 4E is a size distribution graph showing stability data of YF nanoparticles. DLS studies indicated that YF nanoparticles exhibited significantly greater stability (at least about 5 days or more) relative to FF nanoparticles (˜24 hours).

FIG. 5 is a size distribution graph showing stability data of Y nanoparticles. Constructs Y can self-assemble into particles with similar size and stability (at least up to 5 days) as compared to YF nanoparticles.

FIGS. 6A-6E are SEM images of exemplary nanostructures formed from one or more embodiments of the self-assembling peptides described herein. FIG. 6A is a SEM image of YF nanostructures formed from YF peptides at a concentration of about 10 mg/mL in cold water. FIG. 6B is a SEM image of Y nanostructures formed from Y peptides at a concentration of about 5 mg/mL in cold water. FIG. 6C is a SEM image of IL nanostructures formed from IL peptides at a concentration of about 100 mg/mL in cold water. FIG. 6D is a SEM image of IL nanostructures at a lower magnification. FIG. 6E is a SEM image of FF nanostructures formed from FF peptides at a concentration of about 80 mg/mL in cold water. The nanostructures shown in FIGS. 6A-6E were obtained by self-assembly of individual self-assembling peptides followed by flash-freezing and lyophilization or a series of ethanol/hexamethyldisilazane washes (FIG. 6C only) prior to SEM imaging.

FIGS. 7A-7F are characterization data of nanostructures (nanoparticles) showing their sensitivity to various environmental stimuli. FIG. 7A shows that the peptide constructs are environmentally-responsive and a broad range of particle size can be achieved by varying formulation (e.g., concentration and/or construct sequence) and/or processing conditions (e.g., temperature and/or pH). FIG. 7B is a line graph showing effects of pH (e.g., acidic pH vs. basic pH) on the size distribution of YF nanoparticles formed from the YF peptides at a concentration of about 25 mg/mL. FIG. 7C is a line graph showing effects of temperatures (e.g., ranging from about 20° C. to about 45° C.) on the size distribution of FF nanoparticles formed from the FF peptides at a concentration of about 50 mg/mL. FIG. 7D is a line graph showing effects of temperatures on the size distribution of YF nanoparticles formed from the YF peptides at a concentration of about 25 mg/mL. There is a size change with increasing temperature from ˜20° C. to ˜45° C. as measured by dynamic light scattering (DLS). The numeric value (1 or 2) within the parentheses indicated in the figure represents duplicates of the same experiments. In this embodiment, a NaOH solution with a pH of about 8.5 was used as the formulation buffer. FIG. 7E is a line graph showing effects of YF peptide concentration on the size distribution of the resulting YF nanoparticles. FIG. 7F is a line graph showing size distribution of YF nanoparticles (formed from the YF peptides at a concentration of about 10 mg/mL) encapsulating no or an amount of FITC-PEG tagged human serum albumin (HSA). Encapsulation of HSA into the YF nanoparticles resulted in an increase in their hydrodynamic diameters. Triethylamine (TEA)/H₂O was used as the formulation solution to adjust the pH to ˜5.5.

FIGS. 8A-8B are SEM images (at various magnifications) of porous nanoparticles formed by self-assembly of the FF peptides in water, wherein the FF peptides are conjugated to PLGA.

FIG. 9 shows that the hyaluronic acid (HA) gel stiffness can be modulated by temperatures when the HA gel was impregnated with FF nanoparticles.

FIGS. 10A-10B are fluorescent images of YF nanoparticles encapsulating one or more fluorescent dye. FIG. 10A is a fluorescent image of YF nanoparticles (formed from YF peptides at a concentration of about 25 mg/mL) encapsulating calcein dye. FIG. 10B is a set of fluorescent images of YF nanoparticles encapsulating Calcein, a hydrophilic dye (left image in the top and bottom rows) and Nile Red, a hydrophobic dye (center image in the top and bottom rows). The right image in the top and bottom rows of FIG. 10B is a merge image indicating that both dyes were captured by the YF nanoparticles.

FIG. 11 is a line graph showing biodistribution of YF nanoparticles in mice within 2 hours after injection. An imaging dye (e.g., Alexa 750 dye) was encapsulated in the YF nanoparticles and administered to the mice by tail vein injection.

FIG. 12 is a schematic diagram showing temperature-induced particle rearrangement for drug release from FF-based nanoparticles.

FIGS. 13A-13C shows that one or more embodiments of the peptide constructs were conjugated to a nanoparticle (e.g., a gold nanoparticle (AuNP)) and the conjugates were pH-responsive. FIG. 13A is a schematic representation showing preparation of peptide-functionalized AuNPs (e.g., FF-functionalized AuNPs) and aggregation of the peptide-functionalized AuNPs (e.g., FF-functionalized AuNPs) induced by a pH change (e.g., from a pH-6 to a pH-4). FIG. 13B is a set of transmission electron microscopy (TEM) images showing FF-functionalized AuNPs at pH ˜6.0 (left panel) and larger aggregation of the AuNPs as a result of a decrease in pH (e.g., pH ˜4.0) (right panel). FIG. 13C is a line plot of DLS data showing size distribution of FF-functionalized AuNPs (prepared with different coupling molecules such as trityl-S-dPEG®4-acid (dPEG) or alpha lipoic acid(aLA)) at pH ˜6 and pH ˜4-4.5.

FIG. 14 show influence of conservative substitution of at least one residue in the amino acid sequence on size distribution of self-assembled peptide nanoparticles. Each peptide was dissolved in DMSO at ˜380 mg/mL and injected in cold saline solution at ˜2-4° C., resulting in a final peptide concentration of ˜25 mg/mL. Nanoparticles generated from IPGYG peptides were more monodisperse relative to the ones generated from other peptides as indicated in the figure.

FIG. 15 is fluorescent image showing uptake of the peptide nanoparticles described herein by cells. The cells (e.g., NMuMg) were incubated with Alexa 647-loaded peptide nanoparticles and fixed with ˜4% paraformaldehyde. CellMask Green was used to stain the cell mass. Fluorescence imaging was done on a confocal microscope (e.g., a Leica SP5× MP inverted confocal microscope).

FIGS. 16A-16B are graphs showing representative analaytical HPLC traces of purified peptides in accordance with some embodiments described herein. FIG. 16A is a graph showing representative analytical HPLC traces of purified peptide F, FF, Y, YF at ˜210 nm, and ˜254 nm or ˜280 nm using a C18 column. FIG. 16B is a graph showing representative analytical HPLC traces of purified peptide Y and YF at ˜210 nm and ˜280 nm using a C18 column.

FIG. 17 is a plot showing the release kinetics of an agent incorporated into one embodiment of the peptide nanostructures described herein. The calcein dye was incorporated into YF nanoparticles and the release kinetics was measured was measured with the aid of a fluorometer over a period of at least about 45 days.

### DETAILED DESCRIPTION OF THE INVENTION

Provided herein relates to isolated short peptides, and peptide nanostructures that are self-assembled from the short peptides, as well as articles and kits comprising the short peptides and/or self-assembled peptide nanostructures. Methods of forming the peptide nanostructures and using the short peptides and/or peptide nanostructures for various applications are also provided herein. In some embodiments, the short peptides and/or self-assembled nanostructures are stimuli-responsive, e.g., pH-responsive and/or temperature-responsive, and can thus be adapted for various applications such as drug delivery, biotechnology, bioengineering and/or tissue engineering. The inventors have discovered that, in some embodiments, short peptides (e.g., as short as 5 amino acid residues in length such as about 5-10 amino acid residues in length) can spontaneously self-assemble in aqueous media to form discrete spherical particles, for example, with a size in a range of about 50 nm to about 2 μm. The spherical particles can be polydisperse or monodisperse. In some embodiments, the stability of the peptide nanostructures can be tunable—for example, from hours to days to weeks to months—without the need for excipients, stabilizers, and/or crosslinkers. In addition, the inventors have demonstrated that, in some embodiments, the short peptides can be modified, for example, for conjugation to an agent or a substrate, such as a polymer, a protein, or a nanoparticle. In some embodiments where the agent is a peptide-based agent, unitary peptide nanostructures, rather than nanoparticles that are formed and later covalently modified, can be generated. The inventors have also demonstrated the versatility of the short peptides to form different sizes and/or shapes of nanostructures, including but not limited to nanospheres, nanovesicles, nanorods, nanotubes, and nanofibers, based on different formulation and/or processing conditions.

### Isolated Peptides/Self-Assembling Peptides/Peptide Constructs

One aspect provided herein relates to isolated peptides (also termed “self-assembling peptides” or “peptide constructs”, the terms of which are used interchangeably herein). The isolated peptides described herein are synthetic peptides. That is, the isolated peptides described herein are not a product of nature, but, rather, are man-made and do not exist naturally. By way of example only, the isolated peptides can be constructed by any suitable known peptide polymerization techniques, such as solid phase method using standard methods based on either t-butyloxycarbonyl (BOC) or 9-fluorenylmethoxy-carbonyl (FMOC) protecting groups. Additional information about synthesis of the isolated peptides is further described later in the section “Self-assembling peptide synthesis.”

The isolated peptides consists essentially of an amino acid sequence of (Y₁-X₁-X₂-X₃-X₄-X₃-Y₂)ₙ conjugated to at least one entity, wherein X₁ is valine (Val), a substitution thereof and/or a derivative thereof; X₂ is proline (Pro), a substitution thereof and/or a derivative thereof; X₃ is glycine (Gly), a substitution thereof and/or a derivative thereof; X₄ in each n^{th} unit is independently an amino acid residue; and n is an integer from 1 to 50. Stated another way, an isolated peptide comprises (i) an amino acid sequence consisting essentially of (Y₁-X₁-X₂-X₃-X₄-X₃-Y₂)ₙ, and (ii) at least one entity conjugated to the amino acid sequence, wherein X₁ is valine (Val), a substitution thereof and/or a derivative thereof; X₂ is proline (Pro), a substitution thereof and/or a derivative thereof; X₃ is glycine (Gly), a substitution thereof and/or a derivative thereof; X₄ in each n^{th} unit is independently an amino acid residue; and n is an integer from 1 to 50.

The integer n refers to the number of the amino acid sequence unit (Y₁-X₁-X₂-X₃-X₄-X₃-Y₂) present in an isolated peptide described herein. For example, an amino acid sequence consisting essentially of (Y₁-X₁-X₂-X₃-X₄-X₃-Y₂)ₙ, where n=2, refers to an amino acid sequence consisting essentially of (Y₁-X₁-X₂-X₃-X₄-X₃-Y₂-Y₁-X₁-X₂-X₃-X₄-X₃-Y₂), where Y₁'s, X₁'s, X₂'s, X₃'s, and X₄'s can each be independently different or the same.

In some embodiments, n can be an integer from 1 to 25. In other embodiments, n can be an integer from 1 to 10. In some embodiments, n can be an integer from 1 to 4. In some embodiments, n can be an integer from 1 to 3. In other embodiments, n can be an integer from 1 to 2. In one embodiment, n is an integer of 1. In another embodiment, n is an integer of 2. In another embodiment, n is an integer of 3.

Various embodiments of the isolated peptides described herein are able to self-assemble to form a peptide nanostructure described herein and/or induce aggregation of a solid substrate when the solid substrate is functionalized with one or more of the isolated peptides. In some embodiments, the isolated peptides can respond to at least one external stimulus during self-assembly or self-aggregation to form various peptide nanostructures described herein and/or to induce various degrees of aggregation of a solid substrate when the solid substrate is functionalized with one or more of the isolated peptides.

In some embodiments, the isolated peptide consists of an amino acid sequence of (Y₁-X₁-X₂-X₃-X₄-X₃-Y₂)ₙ conjugated to at least one entity, wherein X₁ is valine (Val), a substitution thereof and/or a derivative thereof; X₂ is proline (Pro), a substitution thereof and/or a derivative thereof; X₃ is glycine (Gly), a substitution thereof and/or a derivative thereof; X₄ in each n^{th} unit is independently an amino acid residue; and n is an integer from 1 to 50. Stated another way, in some embodiments, the isolated peptide comprises (i) an amino acid sequence consisting of (Y₁-X₁-X₂-X₃-X₄-X₃-Y₂)ₙ, and (ii) at least one entity conjugated to the amino acid sequence, wherein X₁ is valine (Val), a substitution thereof and/or a derivative thereof; X₂ is proline (Pro), a substitution thereof and/or a derivative thereof; X₃ is glycine (Gly), a substitution thereof and/or a derivative thereof; X₄ in each n^{th} unit is independently an amino acid residue; and n is an integer from 1 to 50.

The term “substitution” when referring to an amino acid residue, refers to a change in an amino acid residue for a different entity, for example another amino acid or amino-acid moiety. Substitutions can be conservative or non-conservative substitutions. In some embodiments, the substitution is a conservative substitution. As used herein, the term “conservative substitution” refers to an amino acid substitution in which the substituted amino acid residue is of similar charge, and/or similar hydrophobicity as the replaced residue. The substituted residue can be of similar size as, or smaller size or larger size than, the replaced residue, provided that the substituted residue has similar biochemical properties (e.g., similar charge and/or hydrophobicity) as the replaced residue. Conservative substitutions of amino acids include, but are not limited to, substitutions made amongst amino acids within the following groups: (i) the small non-polar amino acids: alanine (Ala), methionine (Met), isoleucine (Ile), leucine (Leu), and valine (Val); (ii) the small polar amino acids: glycine (Gly), serine (Ser), threonine (Thr) and cysteine (Cys); (iii) the amido amino acids: glutamine (Gln) and asparagine (Asn); (iv) the aromatic amino acids: phenylalanine (Phe), tyrosine (Tyr) and tryptophan (Trp); (v) the basic amino acids: lysine (Lys), arginine (Arg) and histidine (H); and (vi) the acidic amino acids: glutamine acid (Glu) and aspartic acid (Asp). Substitutions which are charge-neutral and which replace a residue with a similar- or smaller-sized residue can also be considered “conservative substitutions” even if the residues are in different groups (e.g., replacement of phenylalanine with the smaller isoleucine, or replacement of glycine with alanine). The term “conservative substitution” also encompasses the use of amino acid mimetics, analogs, variants, or non-proteinogenic or non-standard amino acid. By way of example only, AdaA or AdaG can be substituted for valine (Val); L-I-thioazolidine-4-carboxylic acid or D-or-L-1-oxazolidine-4-carboxylic acid (See Kauer, U.S. Pat. No. 4,511,390, the content of which is incorporated herein by reference) can be substituted for proline; and Aib, β-Ala, or Acp can be substituted for glycine (Gly).

Accordingly, in some embodiments, X₁ can be valine (Val), or a conservative substitution thereof, e.g., alanine (Ala), methionine (Met), isoleucine (Ile), leucine (Leu) or a derivative thereof. In one embodiment, X₁ is valine or a derivative thereof. In another embodiment, X₁ is alanine (Ala) or a derivative thereof. In another embodiment, X₁ is leucine (Leu) or a derivative thereof. In another embodiment, X₁ is isoleucine (Ile) or a derivative thereof.

In some embodiments, X₂ can be proline (Pro), a conservative substitution and/or a derivative thereof. In one embodiment, X₂ is proline (Pro) or a derivative thereof.

In some embodiments, X₃ can be glycine (Gly), or a conservative substitution thereof, e.g., serine (Ser), threonine (Thr) and cysteine (Cys), alanine (Ala) or a derivative thereof. In one embodiment, X₃ is glycine (Gly) or a derivative thereof. In another embodiment, X₃ is alanine (Ala) or a derivative thereof.

As used herein, the term “derivative” when used in reference to an amino acid residue refers to an amino acid residue derived from a parent amino acid residue, and having a similar structure, charge and/or size as the parent amino acid residue. In some embodiments, the derivative can include a non-proteinogenic amino acid derived from a proteinogenic amino acid. Additional examples of derivatives of an amino acid residue are described in the section “Amino acid residue and exemplary derivatives thereof” in detail later.

Amino Acid Residue X₄:

X₄ can generally be any art-recognized amino acid residue, e.g., a hydrophobic amino acid, a hydrophilic amino acid, or side chain protected hydrophilic amino acid, a proteinogenic amino acid, a non-proteinogenic amino acid, or a derivative thereof, or any amino residue included in the section “Amino acid residue and exemplary derivatives thereof” described later. In some embodiments, at least one or more X₄'s within the amino acid sequence, including at least two X₄'s, at least three X₄'s, at least four X₄'s, and at least five X₄'s or more, can each independently be a hydrophobic amino acid. As used herein, the term “hydrophobic amino acid” refers to an amino acid exhibiting a hydrophobicity of greater than zero according to the normalized consensus hydrophobicity scale of Eisenberg, 1984, J. Mol. Biol. 179:125-142 (1984). Exemplary hydrophobic amino acids include, but are not limited to, Ala, Val, Ile, Leu, Phe, Tyr, Trp, Pro, Met, Gly, and derivatives thereof.

In some embodiments, a hydrophobic amino acid can include an aromatic amino acid. As used herein, the term “aromatic amino acid” refers to a hydrophobic amino acid with a side chain having at least one aromatic or heteroaromatic ring. The aromatic or heteroaromatic ring can contain one or more substituents such as —OH, —SH, —CN, —F, —CI, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₂-C₆) alkyl, (C₂-C₆) alkenyl, substituted (C₂-C₆) alkenyl, (C₂-C₆) alkynyl, substituted (C₂-C₆) alkynyl, (C₅-C2₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Exemplary aromatic amino acids include, but are not limited to, Phe, Tyr and Trp, and derivatives thereof.

In some embodiments, a hydrophobic amino acid can include an aliphatic amino acid. As used herein, the term “aliphatic amino acid” refers to a hydrophobic amino acid having an aliphatic hydrocarbon side chain. Exemplary aliphatic amino acids include, but are not limited to, Ala, Val, Leu and Ile, and derivatives thereof.

In some embodiments, a hydrophobic amino acid can include a nonpolar amino acid. As used herein, the term “nonpolar amino acid” refers to a hydrophobic amino acid having a side chain that is uncharged at physiological pH and which has bonds in which the pair of electrons shared in common by two atoms is generally held equally by each of the two atoms (e.g., the side chain is not polar). Exemplary nonpolar amino acids include, but are not limited to, Leu, Val, Ile, Met, Gly and Ala, and derivatives thereof.

In some embodiments, at least one X₄'s or more within the amino acid sequence, including at least two X₄'s, at least three X₄'s, at least four X₄'s, and at least five X₄'s or more, can each independently be a hydrophilic amino acid. In some embodiments, the hydrophilic amino acid can be charged or uncharged or side-chain modified. As used herein, the term “charged amino acid” refers to an amino acid residue that has a net charge. Accordingly, a charged amino acid can be a cationic amino acid or an anionic amino acid. As used herein, the term “uncharged amino acid” refers to an amino acid residue that has no net charge. A charged amino acid residue can be modified to an uncharged amino acid by masking the charge of the amino acid, for example, by conjugating a protecting group (e.g., a nitrogen-protecting group) to the charge-carrying atom.

In some embodiments, the hydrophilic amino acid can include a polar amino acid. As used herein, the term “polar amino acid” refers to a hydrophilic amino acid having a side chain that is charged or uncharged at physiological pH, but which has at least one bond in which the pair of electrons shared in common by two atoms is held more closely by one of the atoms. Exemplary polar amino acids include, but are not limited to, Asn, Gln, Ser, Thr, and any derivatives thereof.

In some embodiments, the hydrophilic amino acid can include a cationic amino acid. As used herein, the term “cationic amino acid” refers to an amino acid residue that comprises a positively charged side chain under normal physiological conditions. Thus, the term “cationic amino acid” includes any naturally occurring amino acid or mimetic having a positively charged side chain under normal physiological conditions. Generally, amino acid residues comprising an amino group in their variable side chain are considered as cationic amino acids. Exemplary cationic amino acids include, but are not limited to, lysine, histidine, arginine, hydroxylysine, ornithine, and derivatives thereof.

In some embodiments, the hydrophilic amino acid can include an anionic amino acid. As used herein, the term “anionic amino acid” refers to a hydrophilic amino acid having a negative charge. Exemplary anionic amino acids include, but are not limited to, Glu, Asp, and derivatives thereof.

In some embodiments, the hydrophilic amino acid can include an acidic amino acid. As used herein, the term “acidic amino acid” refers to a hydrophilic amino acid having a side chain pK value of less than 7. Acidic amino acids typically have negatively charged side chains at physiological pH due to loss of a hydrogen ion. Exemplary acidic amino acids include, but are not limited to, Glu, Asp, and derivatives thereof.

In some embodiments, the hydrophilic amino acid can include a basic amino acid. As used herein, the term “basic amino acid” refers to a hydrophilic amino acid having a side chain pK value of greater than 7. Basic amino acids typically have positively charged side chains at physiological pH due to association with a hydronium ion. Exemplary basic amino acids include, but are not limited to, His, Arg, Lys, and derivatives thereof.

As will be appreciated by those of skill in the art, as described herein the categories of amino acids are not mutually exclusive. Thus, amino acids having side chains exhibiting two or more physical-chemical properties can be included in multiple categories. For example, amino acid side chains having aromatic moieties that are further substituted with polar substituents, such as Tyr, can exhibit both aromatic hydrophobic properties and polar or hydrophilic properties, and can therefore be included in both the aromatic and polar categories. The appropriate categorization of any amino acid will be apparent to those of skill in the art, especially in light of the detailed disclosure provided herein.

In some embodiments, selection of an amino acid residue (e.g., a hydrophobic amino acid residue) for X₄'s within the peptide sequence can be determined, e.g., based on the self-assembling capability of the isolated peptides to form a peptide nanostructure described herein. In accordance with various aspects described herein, the amino acid residue (e.g., the hydrophobic amino acid residue) at X₄ is selected such that the respective isolated peptides can self-assemble to form a peptide nanostructure described herein. That is, in some embodiments, the isolated peptide excludes the one that is not capable of undergoing self-assembly or self-aggregation to form nanostructures. To determine the self-assembling capability of an isolated peptide, a plurality of the isolated peptides prepared at different concentrations can be subjected to various conditions of forming nanostructures described herein, e.g., in Example 2 or 3, or in the section “Assembly and fabrication of peptide nanostructures.” No detectable nanostructure formed from a mixture of the isolated peptides is indicative of the isolated peptide without any appreciable self-assembling capability.

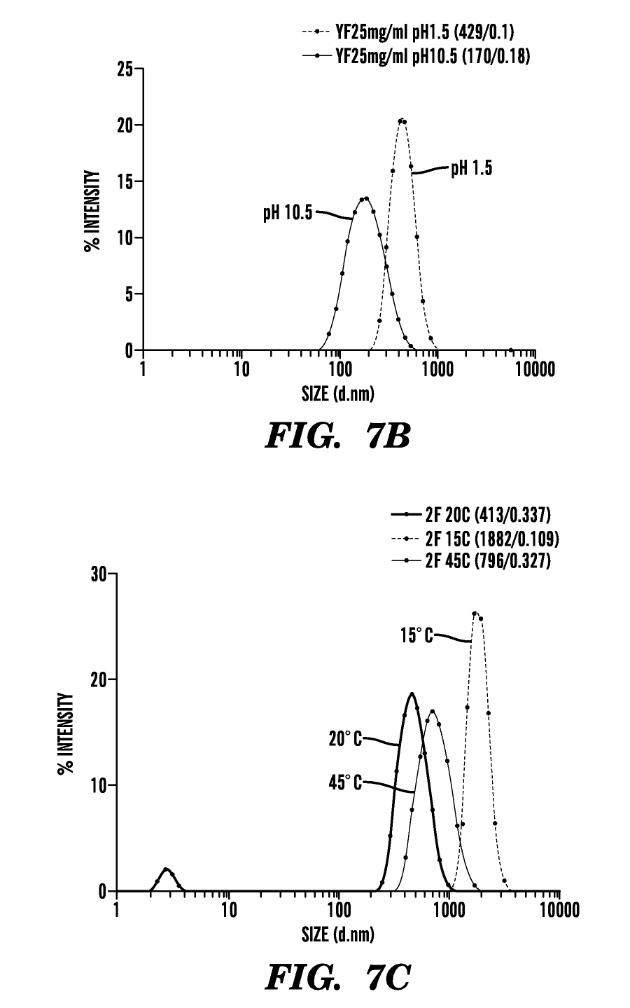

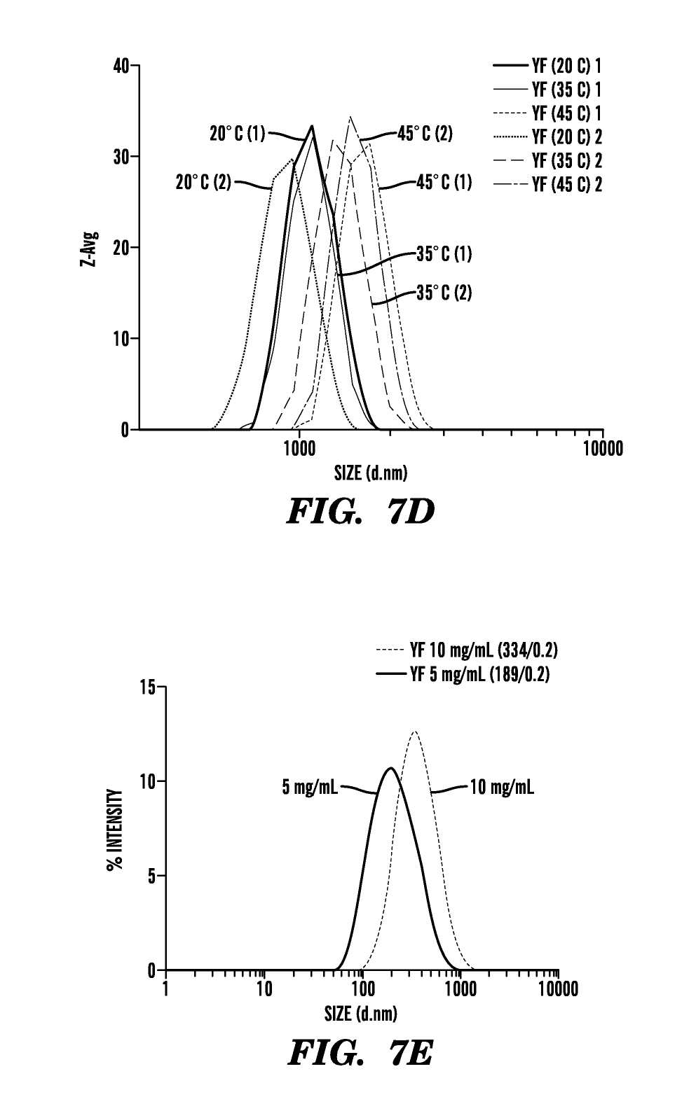

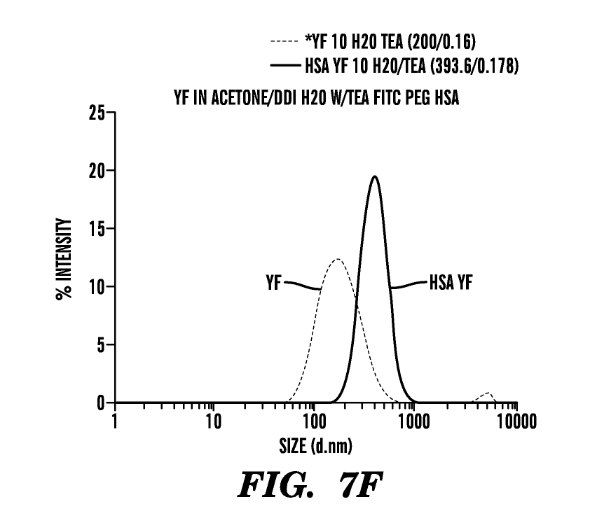

In some embodiments, X₄'s within the peptide sequence can be selected with an amino acid residue that yields an isolated peptide responsive to at least one stimulus, including at least 2 or more stimuli. For example, the size and/shape of the nanostructures formed from the self-assembling peptides described herein can vary depending on the surrounding stimulus or stimuli to which the peptides are exposed. Exemplary stimuli include, but are not limited to, pH, temperature, light, humidity, and a ligand (e.g., but not limited to, a growth factor, a cytokine, and/or a cell surface receptor). In some embodiments, the X₄'s within the peptide sequence can be selected with an amino acid residue that yields an isolated peptide that is responsive to at least temperature, pH, or a combination thereof. For example, FIG. 7B shows different sized nanoparticles formed from one embodiment of the isolated peptides described herein (e.g., YF peptides) at different pHs (e.g., an acidic pH vs. a basic pH), while FIG. 7C shows different sized nanoparticles formed from another embodiment of the isolated peptides described herein (e.g., FF peptides) at various temperatures. Accordingly, nanostructures of different sizes and/or shapes can be formed as a function of various pHs and/or temperatures of the formulation buffer, in which the isolated peptides are dispersed or dissolved during self-assembly.

In some embodiments, X₄'s within the peptide sequence can each be independently selected from the group consisting of phenylalanine (Phe), isoleucine (Ile), leucine (Leu), tyrosine (Tyr), tryptophan (Trp), valine (Val), lysine (Lys), histidine (His), methionine (Met), a non-standard amino acid, and a side-chain modified amino acid. Examples of the non-standard amino acid or side-chain modified amino acid that can be selected for X4 includes, but are not limited to, 4-benzoylphenylalanine (Bpa), 8-hydroxylysine (Hyl), 4-hydroxyproline (Hyp), allo-isoleucine (alle), lanthionine (Lan), β-homoalanine (βHal), β-homoarginine (βHar), β-homoasparagine (βHas), β-homocysteine (βHcy), β-homoglutamine (βHgl), β-homohistidine (βHhi), β-homoisoleucine (βHil), β-homoleucine (βHle), β-homolysine (βHly), β-homomethionine (βHme), β-homophenylalanine (βHph), β-homoproline (βHpr), β-homoserine (βHse), β-homothreonine (βHth), β-homotryptophane (βHtr), β-homotyrosine (βHty), β-homovaline (βHva), substituted phenylalanine (e.g., phenylalanine with a substituted phenyl group, but not limited to, fluoro-phenylalanine, chloro-phenylalanine, bromo-phenylalanine, iodo-phenylalanine, cyan-phenylalanineo, borono-phenylalanine), and any combinations thereof.

In some embodiments where n is 4, at least one X₄, including at least two X₄'s, at least three X₄'s or more, is not valine. In other embodiments where n is 1, X₄ is not valine. In other embodiments where n is 2, at least one of the X₄'s is not valine.

In some embodiments, each of the X₄ within the amino acid sequence is not valine. Accordingly, in such embodiment, X₄'s within the peptide sequence can each be independently selected from the group consisting of phenylalanine (Phe), isoleucine (Ile), leucine (Leu), tyrosine (Tyr), tryptophan (Trp), lysine (Lys), histidine (His), methionine (Met), a non-standard amino acid and a side-chain modified amino acid.

In some embodiments, the X₄'s within the peptide sequence can all correspond to the same amino acid residue. In other embodiments, at least one of the X₄'s, including at least two X₄'s, at least three X₄'s, at least four X₄'s, and at least five X₄'s or more, within the peptide sequence is distinct from the other X₄'s. In one embodiment, each of the X₄'s within the peptide sequence is a distinct amino acid residue.

In some embodiments, the amino acid sequence of the isolated peptide described herein is not a repeated sequence of (VPGVG).

Linkers Y₁ and Y₂:

In embodiments of the isolated peptide described herein, Y₁ and Y₂ are each independently a linker. As used herein, the term “linker” generally means a moiety that is capable of connecting or being modified to connect one molecule, compound or material to another molecule, compound or material. If a linker is located at a terminus of the peptide sequence described herein which is not conjugated to an entity described herein, one of skill in the art will appreciate that the linker can be a null or absent. In some embodiments, two molecules, compounds and/or materials can be linked together by providing on each of the molecules, compounds and/or materials complementary chemical functionalities that undergo a coupling reaction. As used herein, the term “linker” also include non-covalent coupling of two molecules, compounds, and/or materials. Such non-covalent coupling can be achieved through, for example, ionic interactions, H-bonding, van der Waals interactions and affinity of one molecule for another. When non-covalent coupling is used between two molecules, compounds and/or materials, a first molecule, compound and/or material can be conjugated with a moiety that is complementary to another moiety conjugated to a second molecule, compound and/or material. One example of such complementary coupling is the biotin/avidin coupling. Other examples include, affinity of an oligonucleotide for its complementary strand, receptor/ligand binding, aptamer/ligand binding and antibody/antigen binding. This linker can be cleavable or non-cleavable, depending on the application. In certain embodiments, a cleavable linker can be used to release an entity described herein (e.g., but not limited to, a ligand or therapeutic agent) from the peptide sequence conjugated thereto, e.g., after transport to a desired target.

Accordingly, in some embodiments where n is 2 or more, the linkers Y₁ and Y₂ can provide a linkage between any two consecutive amino acid sequence units (-X₁-X₂-X₃-X₄-X₃-) in the isolated peptides described herein. For example, in an amino acid sequence having at least two consecutive amino acid sequence units ( . . . -X₁-X₂-X₃-X₄-X₃-Y₂-Y_{1′}-X_{1′}-X_{2′}-X_{3′}-X_{4′}-X_{3′}- . . . , where the prime symbol (′) in the numeric subscript indicates the residue or the linker is of a different unit), the linkers Y_{1′} and Y₂ can form a linkage of one amino acid residue (e.g., Y_{1′} is a bond while Y₂ is an amino acid residue or vice versa: that is, . . . -X₁-X₂-X₃-X₄-X₃-A-X_{1′}-X_{2′}-X_{3′}-X_{4′}-X_{3′}- . . . , wherein A is an amino acid residue); or a linkage of at least two or more amino acid residue (e.g., Y_{1′} and Y₂ are each independently at least one or more amino acid residues: that is, . . . -X₁-X₂-X₃-X₄-X₃-A₁-A₂- . . . -Aₘ-X_{1′}-X_{2′}-X_{3′}-X_{4′}-X_{3′}- . . . , wherein A₁-A₂- . . . -Aₘ is a series of at least two or more (up to m) consecutive amino acid residues); or a linkage of a molecular bond (i.e., . . . -X₁-X₂-X₃-X₄-X₃-X_{1′}-X_{2′}-X_{3′}-X_{4′}-X_{3′}- . . . ). In some embodiments, the linker Y_{1′} and Y₂ can each be a member of a coupling pair, e.g., but not limited to, biotin/avidin coupling, receptor/ligand binding, aptamer/ligand binding, and antibody/antigen binding. In some embodiments, the linker Y_{1′} and Y₂ can form a non-peptidyl linkage, e.g., but not limited to an oligonucleotide.

In some embodiments, linker Y₁ or Y₂ on at least one terminus (e.g., N-terminus and/or C-terminus) of the amino acid sequence can provide a linkage between the amino acid sequence or isolated peptide and an entity described herein. Depending on types of an entity, the linker Y₁ or Y₂ can include a molecular bond, an amino acid residue, a group of amino acid residues (e.g., 2 or more amino acid residues), a protein molecule, a chemical molecule, a pegylated compound, or any combinations thereof.

In some embodiments, the linker Y₁ or Y₂ present at a free terminus of the isolated peptide (e.g., a N-terminus or a C-terminus that is not conjugated to any entity) can provide at least one site for modification to the terminus of the isolated peptide, e.g., by addition of at least one atom, a functional group, a molecule, and/or at least one amino acid residue to the terminus of the isolated peptide.

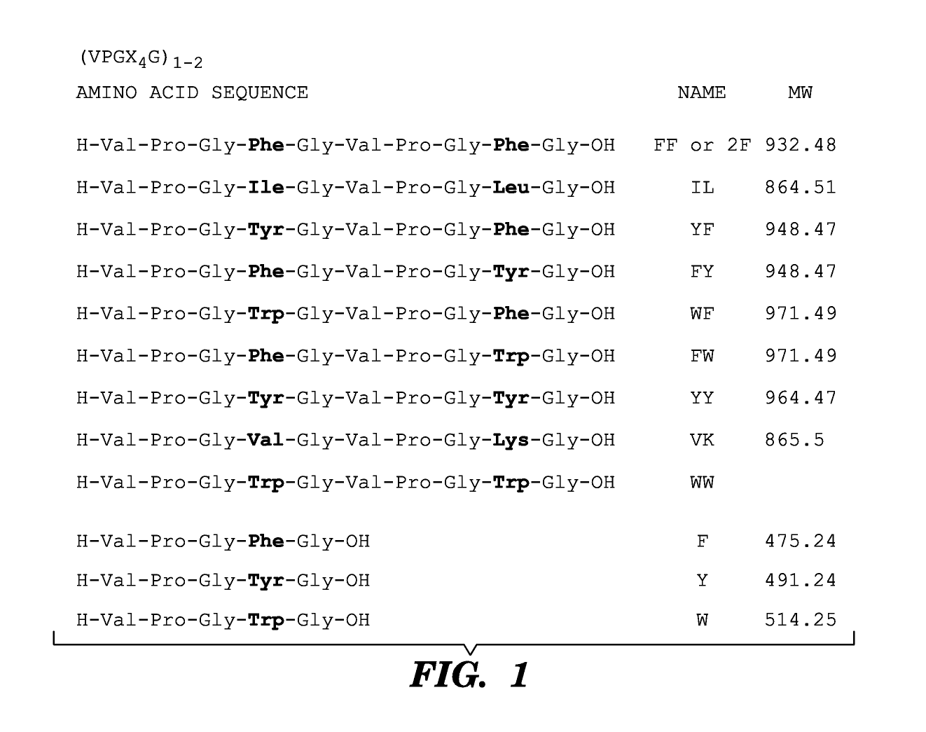

In other embodiments, the linker Y₁ or Y₂ located at an unmodified terminus of the isolated peptide that is not conjugated to an entity can be a part of an amino group (—NH₂) of a N-terminus (e.g., —H of an amino group) or a part of a carboxyl group (—COOH) of a C-terminus (e.g., —OH of a carboxyl group). Accordingly, in these embodiments, the linker Y₁ or Y₂ can be considered as part of the amino group or carboxyl group of the X₁ or X₃ amino acid residue at the terminus, respectively. Stated another way, in such embodiments, the linker Y₁ or Y₂ present on a free, unmodified terminus (e.g., a terminus not conjugated to any entity nor modified) of the isolated peptide can be absent, e.g., the linker is a null. For example, in a FF peptide illustrated in FIG. 1 (i.e., H-V-P-G-F-G-V-P-G-F-G-OH) where the N-terminus of the isolated peptide is considered as a free, unmodified terminus, linker Y₁ is —H of the amino group of the V amino acid residue, and linker Y₂ is a molecular bond conjugated to —OH as an entity. On the other hand, when the C-terminus of the isolated peptide is considered as a free, unmodified terminus, linker Y₂ is —OH of the carboxyl group of the G amino acid residue, and linker Y₁ is a molecular bond conjugated to —H as an entity.

Accordingly, various types of linkers can be used for Y₁ and Y₂, e.g., depending on the position of the Y₁ and Y₂ in the isolated peptide, and/or what the Y₁ and Y₂ being conjugated to. Exemplary linker can include, but is not limited to, a chemical linker (e.g., a molecular bond, an atom, a group of atoms (e.g., 2 or more atoms), a functional group, a molecule, or a compound), a peptidyl linker (e.g., one amino acid residue or a group of amino acid residues (e.g., 2 or more amino acid residues) or a protein molecule), and a combination thereof.

In some embodiments where the linker is a chemical linker, the chemical linker can include an amide linkage (e.g., —NHC(O)—) or an amide replacement linkage, e.g., an amide bond in the backbone replaced by a linkage selected from the group consisting of reduced psi peptide bond, urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, and olefinic group. In some embodiments, the linker can be a direct bond or an atom such as nitrogen, oxygen or sulfur; a unit such as NR₁, C(O), C(O)NH, SO, SO₂, SO₂NH; or a chain of atoms.

In some embodiments, the chemical linker includes a conjugation agent or a cross-linking agent (e.g., a linker used to conjugate an entity to an amino acid construct/sequence described herein). Examples of such conjugation agents or cross-linking agents are described in the section “Conjugation of an entity to an amino acid construct/sequence” below.

In some embodiments where the linker is a peptidyl linker, the peptidyl linker can include one amino acid residue, two amino acid residues, three amino acid residues, four amino acid residues or a non-elastin-based peptide (e.g., non-VPGX₄G-based) comprising from 5 to 20 amino acids. In some embodiments, the peptidyl linker can comprise one or more of the peptide modifications described herein, e.g., amide replacement linkage, beta-amino acids, D-amino acids, chemically modified amino acids, and any combinations thereof.

In some embodiments, for example, where Y₁ and Y₂ serve as peptidyl linkers between two consecutive amino acid sequence units (X₁-X₂-X₃-X₄-X₃), the sum of Y₁ and Y₂ can have no more than 4 amino acid residues, e.g., 4 amino acid residues, 3 amino acid residues, 2 amino acid residues, or 1 amino acid residue. In some embodiments, for example, where Y₁ and Y₂ serve as peptidyl linkers between two consecutive amino acid sequence units (X₁-X₂-X₃-X₄-X₃), the sum of Y₁ and Y₂ can have more than 4 amino acid residues, e.g., 5 amino acid residues, 6 amino acid residues, 7 amino acid residues, 8 amino acid residues, 9 amino acid residues or 10 amino acid residues or more, wherein the combined amino acid sequence of Y₁ and Y₂ cannot comprise a sequence of VPGX₄G or a repeating unit thereof.

The C-terminus of an isolated peptide can be unmodified or modified by conjugating a carboxyl protecting group or an amide group. Exemplary carboxyl protecting groups include, but are not limited to, esters such as methyl, ethyl, t-butyl, methoxymethyl, 2,2,2-trichloroethyl and 2-haloethyl; benzyl esters such as triphenylmethyl, diphenylmethyl, p-bromobenzyl, o-nitrobenzyl and the like; silyl esters such as trimethylsilyl, triethylsilyl, t-butyldimethylsilyl and the like; amides; and hydrazides. Other carboxylic acid protecting groups can include optionally protected alpha-amino acids which are linked with the amino moiety of the alpha-amino acids.

In accordance with some embodiments described herein, the isolated peptide is a hydrophobic peptide. As used herein, the term “hydrophobic peptide” refers to a peptide having a relatively high content of hydrophobic amino acids. In some embodiments, the hydrophobic peptide can behave as an amphiphilic peptide, but they are not classical amphiphilic constructs. Instead, these hydrophobic peptide constructs described herein can have sufficient functional groups such as free N- and C-termini and the amide backbone for capturing hydrophilic materials or compounds and the hydrophobic side chains for capturing hydrophobic materials or compounds. For example, in some embodiments, a peptide can include hydrophilic amino acids described earlier.

Without wishing to be limited, in some embodiments, design and/or optimization of an isolated peptide or a stimulus-responsive isolated peptide with an amino acid sequence including selection of an appropriate amino acid residue for X₄ to form a desired nanostructure can be facilitated and/or predicted using computational simulation. For example, thermodynamic properties of amino acid residues can be generally computed based on their chemical structures and/or charges. Thus, a mathematical algorithm can be used to model and assess the thermodynamic properties associated with conformational changes of the isolated peptides during a self-assembly process and to calculate the free energy of the self-assembly system. See, for example, Wolf M. G. et al. “Rapid Free Calculation of Peptide Self-Assembly by REMD Umbrella Sampling” J. Phys. Chem. B (2008) 112: 13493-13498; and Colombo G. et al. “Peptide Self-Assembly at the Nanoscale: a Challenging Target for Computational and Experimental Biotechnology” for computational methods to model and/or compute free energy of a self-assembly system.

### Entity Conjugated to the Amino Acid Construct/Sequence (Y₁-X₁-X₂-X₃-X₄-X₃-Y₂)ₙ

A wide variety of entities can be coupled to the amino acid construct/sequence described herein. In some embodiments, an entity can alter the distribution, targeting, lifetime, or self-assembly of the isolated peptide or a nanostructure made therefrom. In some embodiments, an entity can provide an additional property or function. For example, in one embodiment, an entity can provide an enhanced affinity for a selected target, e.g., molecule, cell or cell type, compartment, e.g., a cellular or organ compartment, tissue, organ or region of the body, as, e.g., compared to a species absent such a ligand. In another embodiment, a labeling entity, e.g., an imaging agent or dye such as a fluorescent molecule or optical reporter, or a nucleic acid barcode, can facilitate detection and/or imaging of the isolated peptide. In one embodiment, a magnetic entity, e.g., a magnetic particle, can permit detection or isolation of the isolated peptide from a sample with the aid of a magnetic field or magnetic field gradient. In another embodiment, a therapeutic agent can be conjugated to the amino acid construct/sequence as an entity described herein. In some embodiments, an entity can be used as a substrate or solid support, e.g., a particle, to permit conjugation of at least one or a plurality of the amino acid constructs conjugated thereto.

In some embodiments, an amino acid construct described herein can be conjugated to at least one or more (e.g., 1, 2, 3, 4, 5 or more) entities described herein. For example, in some embodiments, a first entity can act as a linker or conjugation or crosslinking agent described herein, e.g., facilitating conjugation of the amino acid construct directly or indirectly to a second entity described herein, e.g., but not limited to, a ligand, a therapeutic agent, and a substrate. In some embodiments, depending on the types of the linker or conjugation or crosslinking agent used as the first entity, a plurality of the amino acid constructs (e.g., at least 2 or more) can be directly or indirectly conjugated to at least one or more (e.g., 1, 2, 3, 4, 5 or more) second entities described herein. By way of example only, a particle as a first entity can not only allow conjugation one or a plurality of the amino acid constructs described herein, but can also provide capability of the amino acid constructs to conjugate to a second entity (e.g., but not limited to a labeling agent) via the first entity, e.g., the particle.

Accordingly, an entity described herein can be any agent, atom, molecule, chemical functional group, compound, material, or substrate that can be conjugated to an amino acid construct described herein by any known methods in the art. Examples of an entity that can be conjugated to the amino acid construct/sequence can include, without limitations, —H, —OH, an atom, a chemical functional group, a ligand, a therapeutic agent, a binding molecule, a coupling molecule, a peptide-modifying molecule, a labeling agent, a substrate, and any combinations thereof.

In some embodiments, an entity can include a —H or —OH. A person of ordinary skill in the art will readily understand that such embodiments can correspond to an isolated peptide consisting essentially of an amino acid sequence of (Y₁-X₁-X₂-X₃-X₄-X₃-Y₂)ₙ without a modification to the N- and C-termini, e.g., the isolated peptides shown in FIG. 1. In these embodiments, the linker Y₁ or Y₂ associated with the entity can be a molecular bond. Stated another way, in these embodiments, the entity (and the associated linker Y₁ or Y₂) can also be considered as part of an amine group (—NH₂) of X₁ at the N-terminus or a carboxyl group —COOH) of X₃ at the C-terminus of the isolated peptide, where the entity (and the associated linker Y₁ or Y₂) can appear to be a null or absent.

In some embodiments, an entity can include a chemical functional group, a linker described herein (e.g., a linker that can be used for Y₁ and Y₂ as described earlier), and/or a conjugation or crosslinking agent described herein. Any chemical functional group, linker, and/or a conjugation or crosslinking agent can be conjugated to the amino acid construct/sequence described herein by various methods known in the art. Non-limiting examples of such chemical function groups can include alkyne, halogens, alcohol, ketone, aldehyde, acyl halide, carbonate, carboxylate, carboxylic acid, ester, hydroperoxide, peroxide, ether, hemiacetal, hemiketal, acetal, ketal, acetal, orthoester, amide, amines, imine, imide, azide, azo compound, cyanates, maleimide, nitrate, nitrile, nitrite, nitro compound, nitroso compound, pyridine, thiol, sulfide, disulfide, sulfoxide, sulfone, sulfinic acid, sulfonic acid, sulfhydryl, thiocyanate, thione, thial, phosphine, phosphonic acid, phosphate, phosphodiester, boronic acid, boronic ester, borinic acid, borinic ester, and any combinations thereof. In some embodiments, the chemical functional group can facilitate the linkage of an amino acid construct/sequence described herein to a molecule, a compound, or another type of entity described herein such as a substrate or a ligand.

In some embodiments, an entity can include a ligand. Ligands can include naturally occurring molecules, or recombinant or synthetic molecules. Non-limiting examples of a ligand can include a cell surface receptor ligand, a targeting ligand, an antibody or a portion thereof, an antibody-like molecule, an enzyme, an antigen, an active agent, a small molecule, a protein, a peptide, a peptidomimetic, a carbohydrate (e.g., but not limited to, monosaccharides, disaccharides, trisaccharides, oligosaccharides, polysaccharides, and lipopolysaccharides), an aptamer, a cytokine, a lectin, a lipid, a plasma albumin, and any combinations thereof. As used herein, the term “targeting ligand” refers to a molecule that binds to or interacts with a target molecule. Typically the nature of the interaction or binding is noncovalent, e.g., by hydrogen, electrostatic, or van der Waals interactions, however, binding can also be covalent.

In some embodiments, a ligand can include an active agent. As used herein and throughout the specification, an “active agent” refers to a molecule that is to be delivered to a cell or to a target area. Accordingly, without limitation, an active agent can be selected from the group consisting of small organic or inorganic molecules, plasmids, vectors, monosaccharides, disaccharides, trisaccharides, oligosaccharides, polysaccharides, biological macromolecules, e.g., peptides, proteins, peptide analogs and derivatives thereof, peptidomimetics, nucleic acids (e.g., but not limited to, DNA, RNA, mRNA, tRNA, RNAi, siRNA, microRNA, or any other art-recognized RNA or RNA-like molecules), nucleic acid analogs and derivatives, polynucleotides, oligonucleotides, enzymes, antibiotics, an extract made from biological materials such as bacteria, plants, fungi, or animal cells or tissues, naturally occurring or synthetic compositions, therapeutic agents, preventative agents, diagnostic agents, imaging agents, antibodies or portions thereof, antibody-like molecules, aptamers (e.g., nucleic acid or protein aptamers) or any combinations thereof. In some embodiments, an active agent can include a biological cell. An active agent can be charge neutral or comprise a net charge, e.g., active agent is anionic or cationic. Furthermore, an active agent can be hydrophobic, hydrophilic, or amphiphilic. In some embodiments, the active agent is biologically active or has biological activity. As used herein, the term “biological activity” or “bioactivity” refers to the ability of a compound to affect a biological sample. Biological activity can include, without limitation, elicitation of a stimulatory, inhibitory, regulatory, toxic or lethal response in a biological assay at the molecular, cellular, tissue or organ levels. For example, a biological activity can refer to the ability of a compound to exhibit or modulate the effect/activity of an enzyme, block a receptor, stimulate a receptor, modulate the expression level of one or more genes, modulate cell proliferation, modulate cell division, modulate cell morphology, or any combination thereof. In some instances, a biological activity can refer to the ability of a compound to produce a toxic effect in a biological sample, or it can refer to an ability to chemical modify a target molecule or cell.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

As used herein, the term “peptidomimetic” refers to a molecule capable of folding into a defined three-dimensional structure similar to a natural peptide.

The term “nucleic acids” used herein refers to polymers (polynucleotides) or oligomers (oligonucleotides) of nucleotide or nucleoside monomers consisting of naturally occurring bases, sugars and intersugar linkages. The term “nucleic acid” also includes polymers or oligomers comprising non-naturally occurring monomers, or portions thereof, which function similarly. Exemplary nucleic acids include, but are not limited to, deoxyribonucleic acid (DNA), ribonucleic acid (RNA), locked nucleic acid (LNA), peptide nucleic acids (PNA), mRNA, tRNA, RNAi, microRNA, and polymers thereof in either single- or double-stranded form. Locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo conformation. LNA nucleotides can be mixed with DNA or RNA residues in the oligonucleotide whenever desired. Such LNA oligomers are generally synthesized chemically. Peptide nucleic acid (PNA) is an artificially synthesized polymer similar to DNA or RNA. DNA and RNA have a deoxyribose and ribose sugar backbone, respectively, whereas PNA's backbone is composed of repeating N-(2-aminoethyl)-glycine units linked by peptide bonds. PNA is generally synthesized chemically. Unless specifically limited, the term “nucleic acids” encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides. In some embodiments, the term “nucleic acid” can encompass modified nucleic acid molecules, such as modified RNA.

The term “enzymes” as used here refers to a protein molecule that catalyzes chemical reactions of other substances without it being destroyed or substantially altered upon completion of the reactions. The term can include naturally occurring enzymes and bioengineered enzymes or mixtures thereof. Examples of enzyme families include kinases, dehydrogenases, oxidoreductases, GTPases, carboxyl transferases, acyl transferases, decarboxylases, transaminases, racemases, methyl transferases, formyl transferases, and α-ketodecarboxylases.

The term “carbohydrate” is used herein in reference to a carbohydrate-based ligand having an affinity for a given cell receptor, such as a carbohydrate-binding protein or an enzyme, and is composed solely or partially of carbohydrate or sugar moieties. In some embodiments, a carbohydrate ligand can be specific for MHC molecules. In some embodiments, a carbohydrate ligand can be specific for a microbe (e.g., virus or bacteria).

As used herein, the term “aptamers” means a single-stranded, partially single-stranded, partially double-stranded or double-stranded nucleotide sequence capable of specifically recognizing a selected non-oligonucleotide molecule or group of molecules. In some embodiments, the aptamer recognizes the non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” can also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

In some embodiments, the ligand can include a cell surface receptor ligand. As used herein, a “cell surface receptor ligand” refers to a molecule that can bind to the outer surface of a cell. Exemplary, cell surface receptor ligand includes, for example, a cell surface receptor binding peptide, a cell surface receptor binding glycopeptide, a cell surface receptor binding protein, a cell surface receptor binding glycoprotein, a cell surface receptor binding organic compound, and a cell surface receptor binding drug. Additional cell surface receptor ligands include, but are not limited to, cytokines, growth factors, hormones, antibodies, and angiogenic factors.

In some embodiments, the ligand can include a targeting ligand. Ligands providing enhanced affinity for a selected target are termed targeting ligands herein. As used herein, the term “targeting ligand” refers to a molecule that binds to or interacts with a target molecule. Typically the nature of the interaction or binding is noncovalent, e.g., by hydrogen, electrostatic, or van der Waals interactions, however, binding may also be covalent.

In some embodiments, the ligand can include an endosomolytic ligand, a PK modulating ligand and/or a PK modulator. As used herein, the term “endosomolytic ligand” refers to molecules having endosomolytic properties. Endosomolytic ligands promote the lysis of and/or transport of the isolated peptide or nanostructure described herein, or its components, from the cellular compartments such as the endosome, lysosome, endoplasmic reticulum (ER), golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

As used herein, the terms “PK modulating ligand” and “PK modulator” refers to molecules which can modulate the pharmacokinetics of the isolated peptide and/or self-assembled nanostructure described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, linear and branched PEGs, biotin, and transthyretia-binding ligands (e.g., tetraiidothyroacetic acid, 2,4,6-triiodophenol and flufenamic acid).

In some embodiments, the entity includes a therapeutic agent. As used herein, the term “therapeutic agent” refers to a biological or chemical agent used for treatment, curing, mitigating, or preventing deleterious conditions in a subject. In some embodiments, the term “therapeutic agent” also encompasses any preventive or prophylactic agent. The term “therapeutic agent” also includes substances and agents for combating a disease, condition, or disorder of a subject, and includes drugs, diagnostics, and instrumentation. “Therapeutic agent” also includes anything used in medical diagnosis, or in restoring, correcting, or modifying physiological functions. The terms “therapeutic agent” and “pharmaceutically active agent” are used interchangeably herein.

A therapeutic agent can be selected according to the treatment objective and biological action desired. Thus, a therapeutic agent can be selected from any class suitable for the therapeutic objective. Further, the therapeutic agent may be selected or arranged to provide therapeutic activity over a period of time.

Exemplary pharmaceutically active compound include, but are not limited to, those found in Harrison's Principles of Internal Medicine, 13^{th} Edition, Eds. T. R. Harrison McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

Exemplary pharmaceutically active agents include, but are not limited to, steroids and nonsteroidal anti-inflammatory agents, antirestenotic drugs, antimicrobial agents, angiogenic factors, calcium channel blockers, thrombolytic agents, antihypertensive agents, anti-coagulants, antiarrhythmic agents, cardiac glycosides, and the like.

In some embodiments, the therapeutic agent is selected from the group consisting of salicylic acid and derivatives (aspirin), para-aminophenol and derivatives (acetaminophen), arylpropionic acids (ibuprofen), corticosteroids, histamine receptor antagonists and bradykinin receptor antagonists, leukotriene receptor antagonists, prostaglandin receptor antagonists, platelet activating factor receptor antagonists, sulfonamides, trimethoprim-sulfamethoxazole, quinolones, penicillins, doxorubicin, cephalosporin, basic fibroblast growth factor (FGF), acidic fibroblast growth factor, vascular endothelial growth factor, angiogenic transforming growth factor alpha and beta, tumor necrosis factor, angiopoietin, platelet-derived growth factor, dihydropyridines (e.g., nifedipine, benzothiazepines such as dilitazem, and phenylalkylamines such as verapamil), urokinase plasminogen activator, urokinase, streptokinase, angiotensin converting enzyme (ACE) inhibitors, spironolactone, tissue plasminogen activator (tPA), diuretics, thiazides, antiadrenergic agents, clonidine, propanolol, angiotensin-converting enzyme inhibitors, captopril, angiotensin receptor antagonists, losartan, calcium channel antagonists, nifedine, heparin, warfarin, hirudin, tick anti-coagulant peptide, and low molecular weight heparins such as enoxaparin, lidocaine, procainamide, encainide, flecanide, beta adrenergic blockers, propranolol, amiodarone, verpamil, diltiazem, nickel chloride, cardiac glycosides, angiotensin converting enzyme inhibitors, angiotensin receptor antagonists, nitrovasodilators, hypolipidemic agents (e.g., nicotinic acid, probucol, etc.), bile acid-binding resins (e.g., cholestyramine, and fibric acid derivatives e.g., clofibrate), HMG CoA reductase inhibitors, HMG CoA synthase inhibitors, squalene synthase inhibitors, squalene epoxidase inhibitors, statins (e.g., lovastatin, cerivastatin, fluvastatin, pravastatin, simvaststin, etc.), anti-psychotics, SSRIs, antiseizure medication, contraceptives, systemic and local analgesics (chronic pain, bone growth/remodeling factors (osteoblast/osteoclast recruiting and stimulating factors), neurotransmitters (L-DOPA, Dopamine, neuropeptides), emphysema drugs, TGF-beta), rapamycin, naloxone, paclitaxel, amphotericin, Dexamethasone, flutamide, vancomycin, phenobarbital, cimetidine, atenolol, aminoglycosides, hormones (e.g., thyrotropin-releasing hormone, p-nitrophenyl beta-cellopentaosideand luteinizing hormone-releasing hormone), vincristine, amiloride, digoxin, morphine, procainamide, quinidine, quinine, ranitidine, triamterene, trimethoprim, vancomycin, aminoglycosides, and penicillin, and pharmaceutically acceptable salts thereof.

In some embodiments, the therapeutic agent includes a radioactive material. Suitable radioactive materials include, for example, of ⁹⁰yttrium, ¹⁹²iridium, ¹⁹8 gold, ¹²⁵iodine, ¹³⁷cesium, ⁶⁰cobalt, ⁵⁵cobalt, ⁵⁶cobalt, ⁵⁷cobalt, ⁵⁷magnesium, ⁵⁵iron, ³²phosphorous, ⁹⁰strontium, ⁸¹rubidium, ²⁰⁶bismuth, ⁶⁷gallium, ⁷⁷bromine, ¹²⁹cesium, ⁷³selenium, ⁷²selenium, ⁷²arsenic, ¹⁰³palladium, ¹²³lead, ¹¹¹Indium, ⁵²iron, ¹⁶⁷thulium, ⁵⁷nickel, ⁶²zinc, ⁶²copper, ²⁰¹thallium and ¹²³iodine. Without wishing to be bound by a theory, particles comprising a radioactive material can be used to treat diseased tissue such as tumors, arteriovenous malformations, and the like.

In some embodiments, the entity includes a labeling agent (e.g., an agent that can be used to tag or label an atom, a molecule, and/or a compound). In some embodiments, a labeling agent can include an imaging agent or a dye. As used herein, the term “imaging agent” refers to an element or functional group in a molecule that allows for the detection, imaging, and/or monitoring of one or more cells in vitro or in vivo. In some embodiments, the imaging agent can be used to detect and/or monitor the presence and/or progression of a condition(s), pathological disorder(s), and/or disease(s). The imaging agent may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber. Without wishing to be bound by a theory, an imaging agent allows tracking of a composition comprising such an imaging agent.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif.), fluorescein, fluorescein isothiocyanate (FITC), Oregon Green™, rhodamine, Texas red, tetrarhodamine isothiocynate (TRITC), 5-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N,N′-tetramefhyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 3-(-carboxy-pentyl)-3′ethyl-5,5′-dimethyloxacarbocyanine (CyA); 1H,5H,11H,15H-Xantheno[2,3,4-ij:5,6,7-i′j′]diquinolizin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17octahydro-inner salt (TR or Texas Red); BODIPY™ dyes; benzoxadiazoles; stilbenes; pyrenes; and the like. Many suitable forms of these fluorescent compounds are available and can be used.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, morange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include, but are not limited to, ¹¹C, ¹⁴C, ¹³N, ¹⁸F, ¹²³I, ¹²⁴I, and ¹²⁵ I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir. In some embodiments, the radionuclide is bound to a chelating agent or chelating agent-linker attached to the aggregate. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶ Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm ¹⁶⁶, Ho, ¹⁷⁷ Lu, ¹⁸⁶ Re, ¹⁸⁸ Re, ²¹¹ At, ²¹² Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to the particles.

A detectable response generally refers to a change in, or occurrence of, a signal that is detectable either by observation or instrumentally. In certain instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ). In certain other instances, the detectable response the detectable response is radioactivity (i.e., radiation), including alpha particles, beta particles, nucleons, electrons, positrons, neutrinos, and gamma rays emitted by a radioactive substance such as a radionuclide.

Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol, 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, IEEE Transactions on Biomedical Engineering, 48:1034-1041 (2001), and the like. Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled aggregate. Positron emission tomography (PET) is another suitable technique for detecting radiation in a subject.

In some embodiments, the entity conjugated to the amino acid construct/sequence described herein can include a substrate. As used herein, the term “substrate” refers to a molecule, material or substance that can permit conjugation of the amino acid constructs/sequences thereon. For example, the substrate can comprise metal, alloy, polymer, glass, carbon, protein, carbohydrate, or any synthetic or naturally-occurring material that does not induce an adverse or undesirable effect on the amino acid constructs/sequences. The substrate can have any shape, e.g., but not limited to, a particle, a scaffold, a sphere, a prism, a wire, a tube, a fiber, a disc, a film, or any art-recognized shape.

In some embodiments, exemplary substrate can include, but are not limited to, a particle (e.g., a nanoparticle or a microparticle), a metal particle (e.g., a gold particle, a silver particle), a polymeric particle (e.g., a non-amino acid polymeric particle), a magnetic particle, a quantum dot, a fullerene, a carbon tube, a nanowire, a nanofibril, a nanotube, a nanoprism, a glass particle, graphene, and any combinations thereof.

In some embodiments, the substrate can include a protein-based substrate including but not limited to extracellular matrix such as collagen, fibronectin, fibrin, laminin, gelatin, as well as albumin, silk and any combination thereof.

In some embodiments, the substrate can include a carbohydrate-based substrate, e.g., but not limited to, glycosaminoglycan, such as hyaluronan (also called hyaluronic acid or hyaluronate or HA).

In some embodiments, the substrate can include a polymer or a polymeric material. Polymers or polymeric materials include, but are not limited to, those that are biocompatible, including, for example, polymeric sugars, such as polysaccharides (e.g., chitosan) and glycosaminoglycans, (e.g., hyaluronan, chondroitin sulphate, dermatan sulphate, keratan sulphate, heparan sulphate, and heparin) and polymeric proteins, such as fibrin, collagen, fibronectin, laminin, and gelatin.

In some embodiments, the substrate can include a biocompatible, non-biodegradable polymer. Examples of the biocompatible, non-biodegradable polymers include, but are not limited to, polyethylenes, polyvinyl chlorides, polyamides, such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers, such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates, such as ethylene vinyl acetate copolymer, homopolymers and copolymers of acrylates, such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers, such as polytetrafluoroethylene and polyvinyl fluoride, polystyrenes, homopolymers and copolymers of styrene acrylonitrile, cellulose aectates, homopolymers and copolymers of acrylonitrile butadiene styrene, polymethylpentenes, polysulfones, polyesters, polyimides, polyisobutylenes, polymethylstyrenes, polyethylene glycol, and other similar compounds known to those skilled in the art. Other biocompatible non-degradable polymers that can be used in accordance with the present disclosure include polymers comprising biocompatible metal ions or ionic coatings. In some embodiments, the substrate can include polyethylene glycol (PEG).

Without limitations, in some embodiments, the substrate can include a non-amino acid polymer. In some embodiments, the substrate can include a biodegradable polymer protein. Exemplary non-amino acid polymer can include, but are not limited to, poly(lactic-co-glycolic acid), poly(ethylene glycol), poly(ethylene oxide), poly(propylene glycol), poly(ethylene oxide-co-propylene oxide), hyaluronic acid, poly(2-hydroxyethyl methacrylate), heparin, polyvinyl(pyrrolidone), chondroitan sulfate, chitosan, glucosaminoglucans, dextran, dextrin, dextran sulfate, cellulose acetate, carboxymethyl cellulose, hydroxyethyl cellulose, cellulosics, poly(trimethylene glycol), poly(tetramethylene glycol), polypeptides, polyacrylamide, polyacrylimide, poly(ethylene amine), poly(allyl amine), and blends thereof. In some embodiments, the substrate can include polyurethanes, polystyrenes, polystyrene sulfonic acid, polystyrene carboxylic acid, polyalkylene oxides, alginates, agaroses, dextrins, dextrans, polyanhydrides, and any combinations thereof. In other embodiments, the substrate can exclude a biodegradable non-amino acid or non-protein polymer.

In some embodiments, the entity conjugated to the amino acid construct/sequence can include a binding molecule or a member of an affinity binding pair or binding pair described herein. By way of example only, an affinity binding pair or binding pair can include biotin-avidin or biotin-streptavidin conjugation. In such embodiments, the entity can include biotin, avidin, streptavidin, immunoglobulin, protein A, protein G, hormone, receptor, receptor antagonist, receptor agonist, enzyme, enzyme cofactor, enzyme inhibitor, a charged molecule, carbohydrate, lectin, steroid, or any combinations thereof.

In some embodiments, the entity conjugated to the amino acid construct/sequence can include a peptide-modifying molecule. As used herein, the term “peptide-modifying molecule” refers to a molecule that can modify at least one property of the isolated peptides or nanostructures made therefrom. In one embodiment, a peptide-modifying molecule can be a molecule that prolongs circulation or plasma half-life of the isolated peptides or nanostructures made therefrom, for example, but not limited to, a polypeptide sequence comprising amino acids Pro, Ala, and Ser (e.g., by PASylation®); a hydroxyethyl starch (HES) derivative (e.g., by HESylation), a PEG molecule (e.g., by PEGylation), and any combinations thereof.

In some embodiments, the entity conjugated to the amino acid construct/sequence can include a coupling molecule or agent. As used herein, the term “coupling molecule” refers a molecule or agent that can be used to link the amino acid construct/sequence to a second entity (e.g., but not limited to a substrate described herein). Examples of a coupling reagent include, but not limited to, any conjugation or crosslinking agent described below, trityl-S-dPEG®4, alpha lipoic acid, and any combinations thereof.

### Conjugation of an Entity to an Amino Acid Construct/Sequence

At least one entity can be conjugated to an amino acid construct/sequence (Y₁-X₁-X₂-X₃-X₄-X₃-Y₂)ₙ of the isolated peptide described herein using any of a variety of methods known to those of skill in the art. The entity can be coupled or conjugated to the amino acid construct/sequence covalently or non-covalently. The covalent linkage between the entity and the amino acid construct/sequence can be mediated by a linker, e.g., linker Y₁ or Y₂, and/or conjugation or crosslinking agent described below. The non-covalent linkage between the entity and the amino acid construct/sequence can be based on ionic interactions, van der Waals interactions, dipole-dipole interactions, hydrogen bonds, electrostatic interactions, and/or shape recognition interactions.

Without limitations, one or more entities (including 1, 2, 3, 4, 5 or more entities) can be coupled to an amino acid construct/sequence at various places, for example, N-terminus, C-terminus, and/or at an internal position (e.g., side chain of an amino acid). In some embodiments, one or more entities can be conjugated to N-terminus of the amino acid construct/sequence. In some embodiments, one or more entities can be conjugated to C-terminus of the amino acid construct/sequence. In some embodiments, when there are two or more entities, they can be placed on opposite ends of an amino acid construct/sequence (e.g., N-terminus and C-terminus).

In some embodiments, the entity can be conjugated or attached to the amino acid construct/sequence via a linker, e.g., a linker Y₁ or Y₂ described herein, and/or a conjugation or crosslinking agent.

As used herein, the term “a conjugation or crosslinking agent” means an organic moiety that connects two parts of a compound. In some embodiments, the terms “conjugation or crosslinking agent” and “linker” are used interchangeably herein. Similar to linkers described herein, a conjugation or crosslinking agent can typically comprise a direct bond or an atom such as oxygen or sulfur, a unit such as NH, C(O), C(O)NH, SO, SO₂, SO₂NH, SS, thiol, sulfhydryl, or a chain of atoms, such as substituted or unsubstituted C1-C6 alkyl, substituted or unsubstituted C2-C6 alkenyl, substituted or unsubstituted C2-C6 alkynyl, substituted or unsubstituted C6-C12 aryl, substituted or unsubstituted C5-C12 heteroaryl, substituted or unsubstituted C5-C12 heterocyclyl, substituted or unsubstituted C3-C12 cycloalkyl, where one or more methylenes can be interrupted or terminated by O, S, S(O), SO2, NH, C(O).

In some embodiments, the conjugation or crosslinking agent is a branched conjugation or crosslinking agent. The branchpoint of the branched conjugation or crosslinking agent can be at least trivalent, but can be a tetravalent, pentavalent or hexavalent atom, or a group presenting such multiple valencies. In some embodiments, the branchpoint is —N, —N(R)—C, —O—C, —S—C, —SS—C, —C(O)N(R)—C, —OC(O)N(R)—C, —N(R)C(O)—C, or —N(R)C(O)O—C; wherein R is independently for each occurrence H or optionally substituted alkyl.

In some embodiments, the conjugation or crosslinking agent comprises a cleavable linking group. As used herein, a “cleavable linking group” is a chemical moiety which is sufficiently stable outside the cell, but which upon entry into a target cell is cleaved to release the two parts the conjugation or crosslinking agent is holding together. In some embodiments, the cleavable linking group is cleaved at least 1.25 times, including at least 1.5 times, at least 2 times, at least 3 times, at least 4 times, at least 5 times, at least 10 times, at least 20 times, at least 30 times, at least 40 times, at least 50 times, at least 100 times or more, faster in the target cell or under a first reference condition (e.g., an in vitro condition which can, e.g., be selected to mimic or represent an intracellular condition) than in the blood or serum of a subject, or under a second reference condition (e.g., an in vitro condition which can, e.g., be selected to mimic or represent an extracellular condition such as a condition found in the blood or serum). In some embodiments, the cleavable linking group is cleaved by less than 90%, 80%, 70%, 60%, 50%, 40%, 30%, 20%, 10%, 5%, 1% or less in the blood or under the second reference condition (e.g., an in vitro condition which can, e.g., be selected to mimic or represent an extracellular condition such as a condition found in the blood or serum) as compared to in the target cell or under the first reference condition (e.g., an in vitro condition which can, e.g., be selected to mimic or represent an intracellular condition).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A conjugation or crosslinking agent can include a cleavable linking group that is cleavable by a particular enzyme. The type of cleavable linking group incorporated into a conjugation or crosslinking agent can depend on the cell to be targeted. For example, for liver targeting, cleavable linking groups can include an ester group. Liver cells are rich in esterases, and therefore the conjugation or crosslinking agent will be cleaved more efficiently in liver cells than in cell types that are not esterase-rich. Other cell-types rich in esterases include cells of the lung, renal cortex, and testis.

Conjugation or crosslinking agents that contain peptide bonds can be used when targeting cell types rich in peptidases, such as liver cells and synoviocytes.

Exemplary cleavable linking groups include, but are not limited to, redox cleavable linking groups (e.g., —S—S— and —C(R)2-S—S—, wherein R is H or C1-C6 alkyl and at least one R is C1-C6 alkyl such as CH3 or CH2CH3); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C1-C10 alkyl); acid cleavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR_{A}C(O)NHCHR_{B}C(O)—, where R_{A} and R_{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleavable in an acidic environment with a pH of about 6.5 or lower (e.g., about 6.0, 5.5, 5.0, or lower), or by agents such as enzymes that can act as a general acid. For example, acid cleavable linking groups can be used for targeting cancer cells where pH within a tumor is generally more acidic than in a normal tissue.

In addition to covalent linkages, two parts of a compound can be linked together by an affinity binding pair. The term “affinity binding pair” or “binding pair” refers to first and second molecules that specifically bind to each other. One member of the binding pair is conjugated with the first part to be linked (e.g., an amino acid construct/sequence described herein) while the second member is conjugated with the second part to be linked (e.g., an entity described herein). As used herein, the term “specific binding” refers to binding of the first member of the binding pair to the second member of the binding pair with greater affinity and specificity than to other molecules.

Exemplary binding pairs include any haptenic or antigenic compound in combination with a corresponding antibody or binding portion or fragment thereof (e.g., digoxigenin and anti-digoxigenin; mouse immunoglobulin and goat antimouse immunoglobulin) and nonimmunological binding pairs (e.g., biotin-avidin, biotin-streptavidin, hormone (e.g., thyroxine and cortisol-hormone binding protein, receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleotide pairs capable of forming nucleic acid duplexes), and the like. The binding pair can also include a first molecule which is negatively charged and a second molecule which is positively charged.

One example of using binding pair conjugation is the biotin-avidin or biotin-streptavidin conjugation. In this approach, one of the molecule or the amino acid construct/sequence is biotinylated and the other (e.g., an entity to be linked) is conjugated with avidin or streptavidin. Many commercial kits are also available for biotinylating molecules, such as proteins or peptides.

Another example of using binding pair conjugation is the biotin-sandwich method. See, e.g., example Davis et al., Proc. Natl. Acad. Sci. USA, 103: 8155-60 (2006). The two molecules to be conjugated together are biotinylated and then conjugated together using tetravalent streptavidin as a linker or conjugation or crosslinking agent.

Still another example of using binding pair conjugation is double-stranded nucleic acid conjugation. In this approach, the first part to be linked (e.g., an amino acid construct/sequence described herein) is conjugated is with linked a first strand of the double-stranded nucleic acid and the second part to be linked (e.g., an entity described herein) is conjugated with the second strand of the double-stranded nucleic acid. Nucleic acids can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges.

A linker or a conjugation or crosslinking agent can be introduced into an isolated peptide by any known methods in the art. For example, a linker or a conjugation or crosslinking agent can be incorporated into an isolated peptide by modifying the first part to be linked (e.g., an amino acid construct/sequence) or the second part to be linked (e.g., an entity) with a coupling agent. Exemplary coupling agent include, without limitations, carbodiimide-based reagents (e.g., but not limited to, dicyclohexylcarbodiimide (DCC), diisopropylcarbodiimide (DIC), and ethyl-(N′,N′-dimethylamino)propylcarbodiimide hydrochloride (EDC)), phosphonium-based reagents (e.g., but not limited to, (benzotriazol-1-yloxy)tris(dimethylamino)phosphonium hexafluorophosphate (BOP), benzotriazol-1-yl-oxytripyrrolidinophosphonium hexafluorophosphate (PyBOP), (7-Azabenzotriazol-1-yloxy)tripyrrolidinophosphonium hexafluorophosphate (PyAOP), bromo-tris-pyrrolidino phosphoniumhexafluorophosphate (PyBroP), and bis(2-oxo-3-oxazolidinyl)phosphonic chloride (BOP-Cl)), aminium-based reagents (e.g., but not limited to, O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazole-1-yl)-N,N,N′,N-tetramethyluronium tetrafluoroborate (TATU), and O-(6-Chlorobenzotriazol-1-yl)-N,N,N′,N′-tetramethyluronium hexafluorophosphate (HCTU)), uronium-based reagents (e.g., but not limited to, O—(N-Succinimidyl)-1,1,3,3-tetramethyl uranium tetrafluoroborate (TSTU), 2-(5-Norborene-2,3-dicarboximido)-1,1,3,3-tetramethyluronium tetrafluoroborate (TNTU), O-(Cyano(ethoxycarbonyl)methylenamino)-1,1,3,3-tetramethyluronium tetrafluoroborate (TOTU), O-(1,2-Dihydro-2-oxo-pyridyl]-N,N,N′,N′-tetramethyluronium tetrafluoroborate (TPTU), and N,N,N′,N-Tetramethyl-O-(3,4-dihydro-4-oxo-1,2,3-benzotriazin-3-yl)uranium tetrafluoroborate(TDBTU)) and any other art-recognized coupling agents (e.g., but not limited to, O-(7-Azabenzotriazole-1-yl)-N,N,N′,N-tetramethyluronium tetrafluoroborate (DEPBT), carbonyldilmidazole (CDI), N,N,N′,N′-tetramethylchloroformamidinium hexafluorophosphate (TCFH), trityl-S-dPEG®4, and alpha lipoic acid).

In some embodiments, the conjugation or crosslinking agent can include a sulfhydryl and/or a thiol. Such conjugation or crosslinking agent can be introduced into an isolated peptide described herein by modifying the first part to be linked (e.g., an amino acid construct/sequence) or the second part to be linked (e.g., an entity) with a coupling reagent, e.g., but not limited to, trityl-S-dPEG®4, alpha lipoic acid, and a combination thereof.

In some embodiments, the conjugation or crosslinking agent can include a maleimide functional group. Such conjugation or crosslinking agent can be introduced into an isolated peptide described herein by modifying the N-terminus of the isolated peptide with a suitable coupling agent, for example, but not limited to, succinimidyl-4-(N-maleimidomethyl)cyclohexane-1-carboxylate (SMCC), N-kappa-Maleimidoundecanoyl-oxysulfosuccinimide ester (KMUS), succinimidyl 6-hydrazinonicotinate acetone hydrazone, SANH (HyNic), succinimidyl 4-formylbenzoate, SFB (S-4FB), and any combinations thereof.

In some embodiments, the entity can be conjugated to the N-terminus of the isolated peptide, e.g., via a linker or by modifying with any art-recognized coupling agent the N-terminus of the isolated peptide, which can then form an amide bond with chemically-activated (e.g., succinimidyl-activated) carboxylic acid on the linker or coupling agent.

### Exemplary Isolated Peptides

In some embodiments, the isolated peptide consists essentially of an amino acid sequence (Y₁-Val-Pro-Gly-X₄-Gly-Y₂)ₙ conjugated to an entity described herein. In some embodiments, the amino acid sequence can include at least one, including at least two, at least three, at least four or more, conservative substitution of any of the subject amino acid residues. In some embodiments where Y₁ and Y₂ are each independently one amino acid residue or a group of amino acid residues, the amino acid residue can include at least one proteinogenic (or standard amino acid) or non-proteinogenic (or non-standard amino acid). In any embodiments described herein, each amino acid residue in the amino acid sequence can be independently a D-amino acid or a L-amino acid.

In some embodiments, the isolated peptide consists essentially of an amino acid sequence (Val-Pro-Gly-X₄-Gly)ₙ conjugated to an entity described herein. In some embodiments, the amino acid sequence can include at least one, including at least two, at least three, at least four or more, conservative substitution of any of the subject amino acid residues. In some embodiments, at least one terminus of the amino acid sequence can be modified, e.g., by addition of an atom or a functional group.

In some embodiments where n is an integer of 2, the isolated peptide described herein has a length of 10 amino acid residues conjugated to an entity. Exemplary 10-amino acid sequences of the isolated peptide can include, but are not limited to,

| | |
|---|---|
| (i) | Val-Pro-Gly-Phe-Gly-Val-Pro-Gly-Phe-Gly; |
| (ii) | Val-Pro-Gly-Ile-Gly-Val-Pro-Gly-Leu-Gly; |
| (iii) | Val-Pro-Gly-Tyr-Gly-Val-Pro-Gly-Phe-Gly; |
| (iv) | Val-Pro-Gly-Phe-Gly-Val-Pro-Gly-Tyr-Gly; |
| (v) | Val-Pro-Gly-Trp-Gly-Val-Pro-Gly-Phe-Gly; |
| (vi) | Val-Pro-Gly-Phe-Gly-Val-Pro-Gly-Trp-Gly; |
| (vii) | Val-Pro-Gly-Tyr-Gly-Val-Pro-Gly-Tyr-Gly; |
| and | |
| (viii) | Val-Pro-Gly-Trp-Gly-Val-Pro-Gly-Trp-Gly. |

Other exemplary 10-amino acid sequence of the isolated peptide can include, but is not limited to, Val-Pro-Gly-Val-Gly-Val-Pro-Gly-Lys-Gly.

In one embodiment, a 10-amino acid sequence of the isolated peptide can include Val-Pro-Gly-Phe-Gly-Val-Pro-Gly-Phe-Gly.

In one embodiment, a 10-amino acid sequence of the isolated peptide can include Val-Pro-Gly-Ile-Gly-Val-Pro-Gly-Leu-Gly.

In one embodiment, a 10-amino acid sequence of the isolated peptide can include Val-Pro-Gly-Tyr-Gly-Val-Pro-Gly-Phe-Gly.

In some embodiments where n is an integer of 1, the isolated peptide described herein has a length of 5 amino acid residues conjugated to an entity. Exemplary 5-amino acid sequences of the isolated peptide can include, but are not limited to,

| | |
|---|---|
| (ix) | Val-Pro-Gly-Phe-Gly; |
| (x) | Val-Pro-Gly-Tyr-Gly; |
| (xi) | Val-Pro-Gly-Trp-Gly; |
| (xii) | Val-Pro-Ala-Tyr-Gly; |
| (xiii) | Ala-Pro-Gly-Tyr-Gly; |
| (xiv) | Ile-Pro-Gly-Tyr-Gly; |
| and | |
| (xv) | Leu-Pro-Gly-Tyr-Gly. |

Other exemplary 5-amino acid sequences of the isolated peptide can include, but are not limited to, Val-Pro-Gly-Leu-Gly and Val-Pro-Gly-Ile-Gly.

In one embodiment, the 5-amino acid sequence of the isolated peptide can include Val-Pro-Gly-Phe-Gly.

In some embodiments, the isolated peptides are elastin-like oligopeptides. Elastin-like polypeptides (ELPs) of more than 200 amino acid residues, in general, are one class of thermoresponsive polymers that are not only temperature-responsive, but also pH- and salt-responsive, in addition to being biocompatible and biodegradable. ELPs are composed of amino acids with the repeating sequence VPGXG, where X can be any amino acid except proline. They are not known to elicit an immunogenic response, and further exhibit a pH-triggered phase transition that controls their shape and mechanical properties (Urry, D. W.; Parker, T. M.; Reid, M. C.; Gowda, D. C. J. Bioact. Compat. Polym. 1991, 6 (3), 263-282). The thermally or pH-triggered phase transition behaviors of ELP pentapeptide repeats can be controlled by the identity of guest residue, X, molecular weight, and concentration (Urry, D. W. J. Phys. Chem. B 1997, 101 (51), 11007-11028; Meyer, D. E.; Chilkoti, A. Biomacromolecules 2004, 5 (3), 846-851). While ELPs are known to self-assemble into nanostructures, there are no identified reports on oligopeptides such as isolated peptides described herein forming nanostructures such as nanospheres.

Surprisingly, in accordance with some embodiments described herein, the isolated peptides can be designed and synthetically sythesized to have a sequence that is up to about 140 times smaller than human tropoelastin, or at least about 5 times (including at least about 10 times, at least about 20 times, or at least about 30 times or higher) smaller than the existing elastin-like polypeptides (ELPs), and yet can spontaneously self-assemble in a formulation medium as described herein to form various forms and/or sizes of nanostructures, e.g., but not limited to nanospheres or microspheres. In some embodiments, these nanostructures can allow encapsulation of an agent of interest (e.g., but not limited to, an active agent, a ligand, a labeling agent, a polymer, or any combinations thereof). In some embodiments, the isolated peptides described herein can encapsulate at least one hydrophobic agent and at least one hydrophilic agent.

In some embodiments, provided herein are novel elastin-based sequences (5-10 amino acids) that can self-assemble into defined nanostructures, including, but are not limited to, nanostructures in a form of a sphere, a capsule, a fiber, a rod, a vesicle, a ring, a disc, a prism, a polygon, or any irregular shape.

### Peptide Nanostructures

Another aspect described herein relates to self-assembled peptide nanostructures comprising a plurality of the isolated peptides described herein. In accordance with some embodiments described herein, the peptide nanostructures are sensitive and/or responsive to at least one (e.g., including at least two or more) external or environmental stimulus, e.g., a particular pH, temperature, light (including a particular wavelength of light), humidity, and/or ionic strength. The response of the peptide nanostructure to the stimulus can be reversible or irreversible. In some embodiments, the response of the peptide nanostructure to the stimulus is reversible. As used herein, the term “reversible” refers to ability of partially or completely reversing or reverting to the original condition (e.g., prior to the exposure of a stimulus) after the change induced by the stimulus.

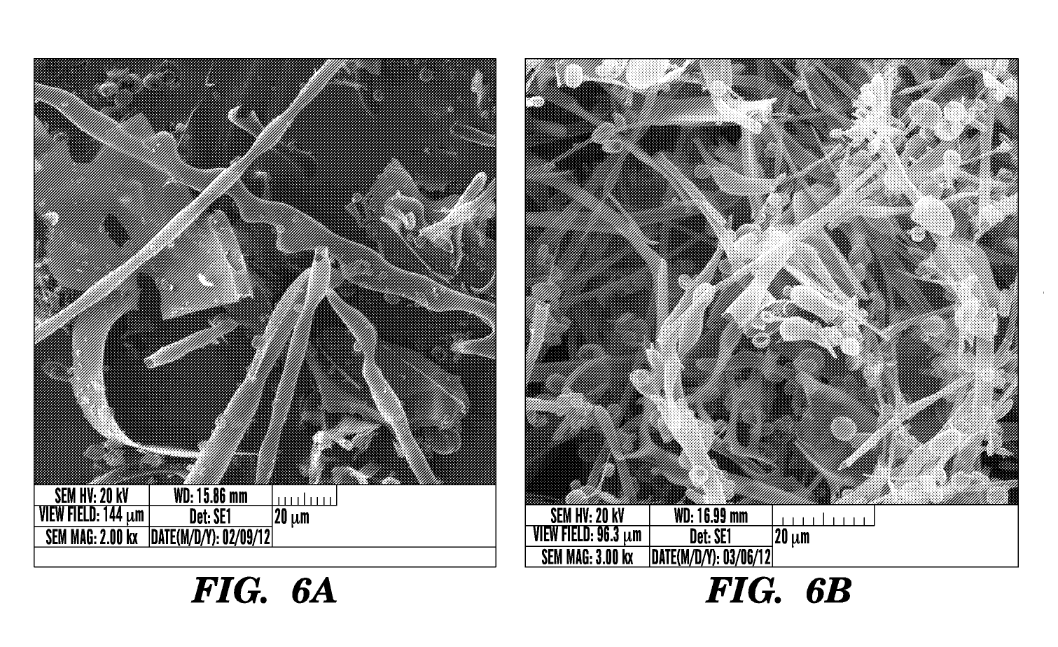

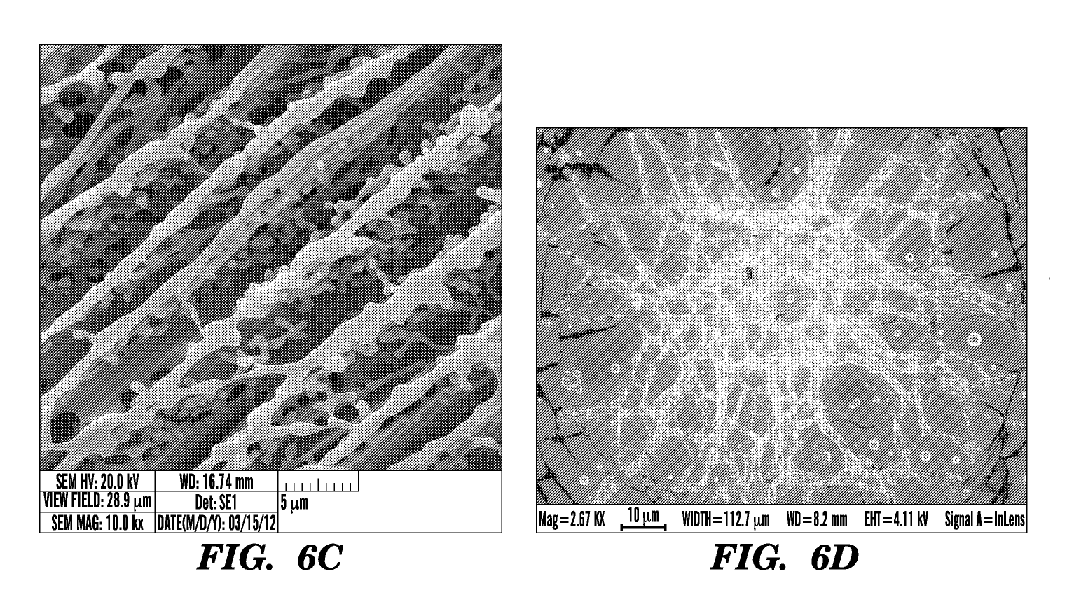

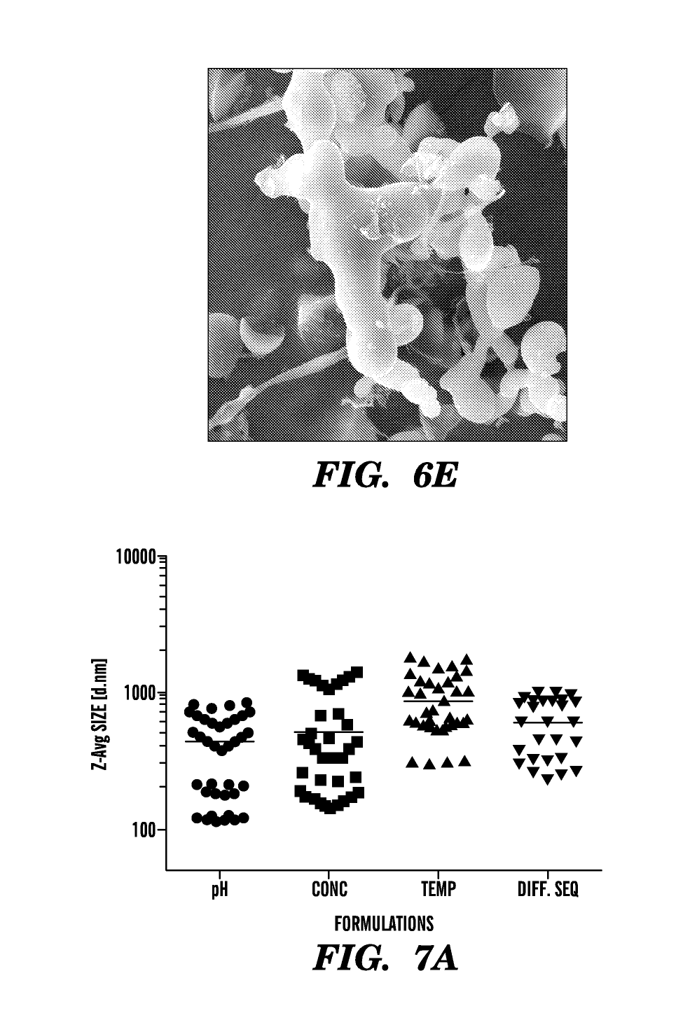

In some embodiments, the peptide nanostructures are temperature-responsive. The term “temperature-responsive” as used in reference to a peptide nanostructure refers to the ability of a peptide nanostructure to change its shape and/or size in response to a change (increase or decrease) in the surrounding temperature. For example, as shown in FIGS. 6A-6B, and 6D-6E, the self-assembled nanoparticles (e.g., nanospheres) from the isolated peptides described herein can self-reassemble into another nanostructure of a different shape and/or form (e.g., but are not limited to, nanovesicles, nanotubes, nanofibers) when they were subjected to flash-freezing followed by lyophilization. In other embodiments, the temperature-responsive peptide nanostructure can change (decrease or increase) its size, e.g., by at least about 10% or more of its original size, without any significant change in its shape and/or form when they are subjected to a change in surrounding temperature. In some embodiments, the temperature-responsive peptide nanostructure can change (decrease or increase) its size, e.g., by at least about 10%, at least about 20%, at least about 30%, at least about 40%, at least about 50%, at least about 60%, at least about 70%, at least about 80%, at least about 90%, at least about 95% or more, of its original size, when the nanostructures are subjected to a change (decrease or increase) in surrounding temperature (e.g., at least about 5° C. change, at least about 10° C. change, at least about 15° C. change, at least about 20° C. change, at least about 25° C. change, at least about 30° C. change, at least about 35° C. change, at least about 40° C. change, at least about 45° C. change, at least about 50° C. change or more). In some embodiments, the temperature-responsive peptide nanostructure can increase or decrease its size, e.g., by at least about 10%, at least about 20%, at least about 30%, at least about 40%, at least about 50%, at least about 60%, at least about 70%, at least about 80%, at least about 90%, at least about 95% or more, of its original size, when the nanostructures are subjected to a change (decrease or increase) in surrounding temperature from about 4° C. to about 50° C., from about 4° C. to body temperature of a subject (e.g., about 37° C. for a human), from about 10° C. to body temperature of a subject (e.g., about 37° C. for a human) or from about room temperature to body temperature of a subject (e.g., about 37° C. for a human).

In some embodiments, the peptide nanostructures are pH-responsive. The term “pH-responsive” as used in reference to a peptide nanostructure refers to the ability of a peptide nanostructure to change its shape and/or size in response to a change (increase or decrease) in the surrounding pH. In some embodiments, a change in the surrounding pH can cause the formed pH-responsive nanostructure to self-reassemble into another shape and/or form. In other embodiments, a change in the surrounding pH can result in a change in size of the formed pH-responsive nanostructure, e.g., by at least about 10% or more of its original size, without any significant change in the original shape/form. In some embodiments, the pH-responsive peptide nanostructure can change (decrease or increase) its size, e.g., by at least about 10%, at least about 20%, at least about 30%, at least about 40%, at least about 50%, at least about 60%, at least about 70%, at least about 80%, at least about 90%, at least about 95% or more, of its original size, when the nanostructures are subjected to a change in surrounding pH (e.g., pH±˜0.5, pH±˜1, pH±˜1.5, pH±˜2, pH±˜2.5, pH±˜3, pH±˜3.5, pH±˜4, pH±˜4.5, pH±˜5, pH±˜5.5, pH±˜6, pH±˜6.5, pH±˜7, pH±˜8, pH±˜9, pH±˜10 or more). In some embodiments, the pH-responsive peptide nanostructure can increase or decrease its size, e.g., by at least about 10%, at least about 20%, at least about 30%, at least about 40%, at least about 50%, at least about 60%, at least about 70%, at least about 80%, at least about 90%, at least about 95% or more, of its original size, when the nanostructures are subjected to a change in surrounding pH from pH˜1 to pH˜14, from pH˜1 to physiological pH (which can vary with tissues and/or organs, e.g., more acidic in stomach than in other tissue generally with a pH˜7), from pH˜4 to physiological pH, from pH˜14 to physiological pH, or from pH˜10 to physiological pH.

As used herein, the term “self-assemble,” or “self-assembly” refers to the ability of self-assembling isolated peptides described herein to form a nanostructure under a specified condition and/or in response to at least an environmental or external stimulus, e.g., a particular pH, temperature, light (including a particular wavelength of light), humidity, and/or ionic strength. Without wishing to be bound by theory, molecular recognition processes are generally involved in ordered assemblies of isolated peptides to form a nanostructure during a self-assembly process. The term “molecular recognition” is used herein in reference to specific interaction during a self-assembly process between two or more isolated peptides, for example, through noncovalent bonding such as hydrogen bonding, metal coordination, hydrophobic forces, van der Waals forces, π-π interactions, electrostatic and/or electromagnetic effects. In some embodiments, the formation of a self-assembled nanostructure can be spontaneous (e.g., the self-assembly process occurs within about 15 minutes, within about 10 minutes, within about 5 minutes or less). In some embodiments, the formation of a self-assembled nanostructure can occur over a longer period of time, for example, over a period of about 30 minutes, about 1 hour, about 2 hours or more. As used herein, the term “self-reassemble” refers to the ability of self-assembling isolated peptides described herein or the formed nanostructures to re-arrange for another nanostructure of different shape and/or size.

The self-assembled peptide nanostructures can be of any shape and/or size depending on the processing conditions and/or formulation condition in which the self-assembling peptides are dispersed or dissolved. For example, the size of the self-assembled peptide nanostructures can be controlled by varying pH, and/or temperatures of the formulation buffer, concentration of the self-assembling peptides present in the formulation buffer, composition of the formulation buffer, and/or types of the entity conjugated to the amino acid construct. As shown in FIG. 7B, with a specified isolated peptide, larger nanostructures (e.g., nanostructures formed from isolated YF peptides with an amino acid sequence displayed in Table 1) were formed at acidic pH (e.g., pH˜1.5) than at basic pH (e.g., pH˜10.5). Lower temperatures (e.g., ˜15° C. or colder) resulted in larger nanostructures (e.g., FF nanostructures) than at higher temperatures (e.g., room temperature or higher) (FIG. 7C). However, for some self-assembling peptide constructs, larger nanostructures (e.g., YF nanostructures) were formed (e.g., in a basic buffer such as NaOH solution with a pH of about 8.5) at higher temperatures than at lower temperatures (FIG. 7D). In some embodiments, as shown in FIGS. 6A-6B, and 6D-6E, different shapes and/or forms of nanostructures can be formed by varying the processing temperature, e.g., subjecting the formed nanospheres to flashing-freezing followed by lyophilization can changes the forms of nanostructures from nanospheres to other forms such as nanofibers, nanovesicles, nanorods, nanotubes and/or nanorings. Accordingly, the effects of external stimuli (e.g., pH and/or temperatures) on size/shape of self-assembled nanostructures can be specific to the amino acid sequence of the isolated peptide.

Self-assembling isolated peptides described herein are also responsive to formulation composition including peptide concentration. For example, FIG. 7E indicates that keeping other conditions constant, higher peptide concentration during a self-assembly process can result in larger nanostructures. In some embodiments, the form/shape of nanostructures can change (e.g., from spheres to rods) when all other processing conditions remain the same but the relative peptide concentrations are significantly higher than or at some critical levels. The critical concentrations of each peptide construct can vary depending on the amino acid sequence of the construct. For example, peptide construct IL at a concentration of about 300 mg/mL can form a different nanostructure as compared to the same peptide construct at a concentration of about 100 mg/mL (Data not shown).

Accordingly, the peptide nanostructures can be present in any form or shape, including but not limited to, a particle, a fiber, a rod, a gel, a tube, a vesicle, a ring, or any combinations thereof. In some embodiments, the peptide nanostructures can be in a form of particles including spheres, discs, prisms, rings, vesciles, rods, fibers, or any irregular-shaped particles.

In some embodiments, the self-assembled peptide nanostructures can have an average size or dimension ranging from nanometers to micrometers, e.g., from about 5 nm to about 500 μm, from about 10 nm to about 250 μm, from about 25 nm to about 100 μm, from about 50 nm to about 50 μm or from about 50 nm to about 3 μm. In some embodiments, the self-assembled peptide nanostructures can have an average size or dimension ranging from about 5 nm to 5000 nm, from about 10 nm to about 2500 nm, from about 25 nm to about 2000 nm, from about 50 nm to about 1000 nm, from about 100 nm to about 500 nm. In some embodiments, the self-assembled peptide nanostructures can have an average size or dimension ranging from about 1 μm to about 500 μm, from about 2 μm to about 250 μm, from about 3 μm to about 100 μm, or from about 5 μm to about 50 μm.

In some embodiments, the self-assembled nanostructures described herein (e.g., self-assembled particles) can be monodisperse (characterized by a relatively low polydispersity index, e.g., less than 0.4 or 40%). Accordingly, in some embodiments, the diameter of a self-assembled particle described herein is generally within ±35%, within ±30%, within ±25%, within ±20%, within ±15%, within ±10%, within ±5%, or within ±2.5% of the average size or diameter described herein.

In some embodiments, the peptide nanostructures can be tuned to be stable over any period of time. As used herein, the term “stable” refers to the property (e.g., size and/or shape) of the nanostructure being maintained (e.g., at least about 70% or more of the original size being maintained) at a certain condition (e.g., a physiological condition) over a specified period of time, e.g., in hours, weeks, or months. For example, a stable peptide nanostructure can maintain its size and/or shape (e.g., at least about 70% or more of the original size being maintained) over a period of at least about 6 hours, at least about 12 hours, at least about 1 day, at least about 2 days, at least about 3 days, at least about 4 days, at least about 5 days, at least about 6 days, at least about 7 days or more. In some embodiments, a stable peptide nanostructure can maintain its size and/or shape (e.g., at least about 70% or more of the original size being maintained) over a period of at least about 1 week, at least about 2 weeks, at least about 3 weeks, at least about 4 weeks, or more. In some embodiments, a stable peptide nanostructure can maintain its size and/or shape (e.g., at least about 70% or more of the original size being maintained) over a period of at least about 1 month, at least about 2 months, at least about 3 months, at least about 4 months, at least about 5 months, at least about 6 months or more.

In some embodiments, the peptide nanostructures can be biodegradable. For example, at least about 5% or more, including at least about 10%, at least about 20%, at least about 30% or more, of the peptide nanostructure can degrade in vivo over a specified period of time, e.g., a period of at least about 1 day, at least about 3 days, at least about 1 week, at least about 2 weeks, at least about 3 weeks, at least about 4 weeks or more. In some embodiments, at least about 5% or more, including at least about 10%, at least about 20%, at least about 30% or more, of the peptide nanostructure can degrade in vivo over a period of at least about 1 month, at least about 2 months, at least about 3 months, at least about 4 months, at least about 5 months, at least about 6 months or more. In some embodiments, the peptide nanostructures can be stable in vivo for a certain period of time before they start to degrade in vivo.

In some embodiments, the peptide nanostructures can be porous. For example, the peptide nanostructure can have a porosity of at least about 30%, at least about 40%, at least about 50%, at least about 60%, at least about 70%, at least about 80%, at least about 90%, or higher. Too high porosity can yield a peptide nanostructure with lower mechanical properties, but with faster release of a therapeutic agent or an active agent encapsulated therein. However, too low porosity can decrease the release of a therapeutic agent or an active agent. One of skill in the art can adjust the porosity accordingly, based on a number of factors such as, but not limited to, desired release rates, molecular size and/or diffusion coefficient of the therapeutic agent or active agent, and/or concentrations and/or amounts of self-assembling peptides in a peptide nanostructure. The term “porosity” as used herein is a measure of void spaces in a material, e.g., a matrix such a peptide nanostructure, and is a fraction of volume of voids over the total volume, as a percentage between 0 and 100% (or between 0 and 1). Determination of matrix porosity is well known to a skilled artisan, e.g., using standardized techniques, such as mercury porosimetry and gas adsorption, e.g., nitrogen adsorption.

The porous peptide nanostructure can have any pore size. In some embodiments, the pores of a peptide nanostructure can have a size distribution ranging from about 50 nm to about 1000 μm, from about 250 nm to about 500 μm, from about 500 nm to about 250 μm, from about 1 μm to about 200 μm, from about 10 μm to about 150 μm, or from about 50 μm to about 100 μm. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the pore size of a self-assembled peptide nanostructure can vary with the amino acid sequence designed for the self-assembling peptide described herein, e.g., due to strength of interaction between the self-assembling peptides to form the nanostructure.

In some embodiments, the peptide nanostructures can have a solid structure. As used herein, the term “solid structure” generally refers to a structure having aggregates or agglomerates of solid matter to occupy the inside volume or core space of the structure. For example, a solid peptide nanostructure can have the isolated peptides described herein occupying at least about 50% or more (including, e.g., at least about 60%, at least about 70%, at least about 80%, at least about 90%, at least about 95% or more) of the inside volume or core space of the nanostructure.

In some embodiments, the peptide nanostructures can have a hollow core structure surrounded by a shell layer. In these embodiments, the isolated peptides described herein can self-assemble to form the shell layer surrounding a hollow space therein.

In some embodiments, the peptide nanostructures can have a lamellar structure. As used herein, the term “lamellar” refers to a structure having at least two layers, including, e.g., at least three layers, at least four layers, at least five layers or more.

The peptide nanostructure described herein can be used as a delivery vehicle. Thus, a wide variety of any active agents as described herein (e.g., but not limited to, therapeutic agents, preventative agents, diagnostic agents, and imaging agents) can be included in the peptide nanostructures described herein. In some embodiments, the active agent(s) can be coated on the peptide nanostructures described herein. In some embodiments, the active agent(s) can be encapsulated inside the peptide nanostructures. For example, to encapsulate the active agent(s) inside the peptide nanostructures, in some embodiments, the active agent(s) can be conjugated to the self-assembling peptides prior to formation to formation of the peptide nano structures. Alternatively or additionally, the active agent(s) can be added to a mixture of the self-assembling peptides during formation of the peptide nanostructures. Accordingly, in some embodiments, a peptide nanostructure described herein can further comprise at least one active agent, including at least two, at least three, at least four, at least five or more active agents as described herein. In some embodiments, the active agent can include one or more cells.

The term “cells” used herein refers to any cell, prokaryotic or eukaryotic, including plant, yeast, worm, insect and mammalian. In one embodiment, the peptide nanostructure can further comprise at least one cell, including at least about 10 cells, at least about 100 cells, at least about 1000 cells, at least about 10⁴ cells, at least about 10⁵ cells, at least about 10⁶ cells or more. In one embodiment, the cell(s) included in the nanostructure described herein can include mammalian cell(s). Mammalian cells include, without limitation; primate, human and a cell from any animal of interest, including without limitation; mouse, hamster, rabbit, dog, cat, domestic animals, such as equine, bovine, murine, ovine, canine, feline, etc. In one embodiment, the mammalian cell is a human cell. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells, induced pluripotent stem cells and stem cell progenitors are also included, including without limitation, hematopoeitic, neural, stromal, muscle, cardiovascular, hepatic, pulmonary, gastrointestinal stem cells, etc. Yeast cells can also be used as cells in some embodiments. In some embodiments, the cells can be ex vivo or cultured cells, e.g. in vitro. For example, for ex vivo cells, cells can be obtained from a subject, where the subject is healthy and/or affected with a disease. Cells can be obtained, as a non-limiting example, by biopsy or other surgical means know to those skilled in the art.

In some embodiments, an active agent (e.g., but not limited to, therapeutic agents, preventative agents, diagnostic agents, and imaging agents) can be covalently linked with a component, e.g., a self-assembling peptide, of the nanostructure. In some embodiments, an active agent (e.g., but not limited to, therapeutic agents, preventative agents, diagnostic agents, and imaging agents) in the particle is not covalently linked to a component of the nanostructure. Without limitations, the active agent (e.g., but not limited to, therapeutic agents, preventative agents, diagnostic agents, and imaging agents) can be absorbed/adsorbed on the surface of the nanostructure, encapsulated in the nanostructure, or distributed (homogenously or non-homogenously) throughout the nanostructure. In one embodiment, at least one (including 1, 2, 3, 4, 5 or more) active agents can be encapsulated in the nanostructure described herein.

Generally, any ratio of active agent or therapeutic agent to isolated peptides described herein can be present in the nanostructure. Accordingly, in some embodiments, ratio of the active agent or therapeutic agent to the self-assembling peptides ranges from about 100:1 to about 1:100,000. In some embodiments, ratio of the active agent or therapeutic agent to the self-assembling peptides ranges from about 1:1 to about 1:100,000. In some embodiments, ratio of the active agent or therapeutic agent to the self-assembling peptides ranges from about 1:1 to about 1:1000. In some embodiments, ratio of the active agent or therapeutic agent to the self-assembling peptides ranges from about 50:1 to about 1:500. In some embodiments, ratio of the active agent or therapeutic agent to the self-assembling peptides ranges from about 10:1 to about 1:25.

In some embodiments, the peptide nanostructures (porous or non-porous) can be used to deliver a therapeutic agent to a target site for treatment of any disease, disorder or injury. In one embodiment, the peptide nanostructures (porous or non-porous) can be used to deliver a therapeutic agent to a target site for treatment of a respiratory disease or lung-related disease or disorder. In some embodiments, the peptide nanostructures (porous or non-porous) can be used as a delivery vehicle for a therapeutic agent to be administered by inhalation. Without wishing to be bound by theory, the aerodynamic diameter (Da) of a drug delivery vehicle is a key attribute that determines its regional deposition in the lung, which in turn affects inhaled drug safety and efficacy. In some embodiments, the porous peptide nanostructures (particularly porous peptide nanoparticles such as nanospheres) can be less dense relative to solid particles and therefore the MMAD (mass median aerodynamic diameter) can be well within the respirable range for targeting local delivery to the lungs as well as systemic delivery by inhalation. Accordingly, the peptide nanoparticles such as nanospheres can likely eliminate the need for expensive spraying approach in aerosol delivery.

In some embodiments, the nanostructure can further comprise a ligand. In some embodiments, the ligand is a targeting ligand. Without limitations, a ligand can be covalently linked with a component, e.g., self-assembling peptides, of the nanostructure. In some embodiments, a ligand is not covalently linked to a component of the nano structure, e.g., the ligand is absorbed/adsorbed on the surface of the nanostructure, the ligand is encapsulated in the nanostructure, or the ligand is distributed (homogenously or non-homogenously) throughout the nanostructure. In some embodiments where the ligand is distributed on the surface of the peptide nanostructure, the peptide nanostructure can be desirable for targeted drug delivery.

Generally, any ratio of ligand to self-assembling peptides can be present in the nanostructure. Accordingly, in some embodiments, ratio of the ligand to the self-assembling peptides ranges from about 1000:1 to about 1:1000. In some embodiments, ratio of the ligand to the self-assembling peptides ranges from about 500:1 to about 1:500. In some embodiments, ratio of the ligand to the self-assembling peptides ranges from about 250:1 to about 1:250. In some embodiments, ratio of the ligand to the self-assembling peptides ranges from about 100:1 to about 1:100. In some embodiments, ratio of the ligand to the self-assembling peptides ranges from about 10:1 to about 1:10.

In some embodiments, a peptide nanostructure can further comprise a polymer, e.g., a biocompatible polymer. The polymer can be conjugated to the peptide nanostructures or be blended with a plurality of the isolated peptides during self-assembly. As used herein, the term “biocompatible” means exhibition of essentially no cytotoxicity or immunogenicity while in contact with body fluids or tissues. As used herein, the term “polymer” refers to oligomers, co-oligomers, polymers and co-polymers, e.g., random block, multiblock, star, grafted, gradient copolymers and combination thereof.

The term “biocompatible polymer” refers to polymers which are non-toxic, chemically inert, and substantially non-immunogenic when used internally in a subject and which are substantially insoluble in blood. The biocompatible polymer can be either non-biodegradable or preferably biodegradable. Preferably, the biocompatible polymer is also noninflammatory when employed in situ.

Biodegradable polymers are disclosed in the art. Examples of suitable biodegradable polymers include, but are not limited to, linear-chain polymers such as polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), and copolymers, terpolymers, and copolymers including one or more of the foregoing. Other biodegradable polymers include, for example, gelatin, collagen, silk, chitosan, alginate, cellulose, poly-nucleic acids, etc.

Suitable non-biodegradable biocompatible polymers include, by way of example, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), poloxomers (e.g. Pluronic such as Poloxamers 407 and 188), Hyaluron, heparin, agarose, Pullulan, and copolymers including one or more of the foregoing, such as ethylene/vinyl alcohol copolymers (EVOH).

The peptide nanostructure can also comprise additional moieties that can extend the lifetime of the particles in vivo. For example, the peptide nanostructure can comprise functional moieties that enhance the in vivo lifetime of the particles in the blood. One exemplary moiety for increasing the in vivo lifetime is polyethylene glycol. Accordingly, in one embodiment, the peptide nanostructure can comprise polyethylene glycol in addition to the self-assembling isolated peptide. In other embodiments, the peptide nanostructure can also be PASylated and/or HASylated to increase its circulation half-time in vivo. For example, in some embodiments, the peptide nanostructure can have a circulation half-time of at least about 4 hours, at least about 6 hours, at least about 12 hours, at least about 24 hours, at least about 2 days, at least about 3 days, at least about 4 days, at least about 5 days, at least about 6 days, at least about 7 days, or longer.

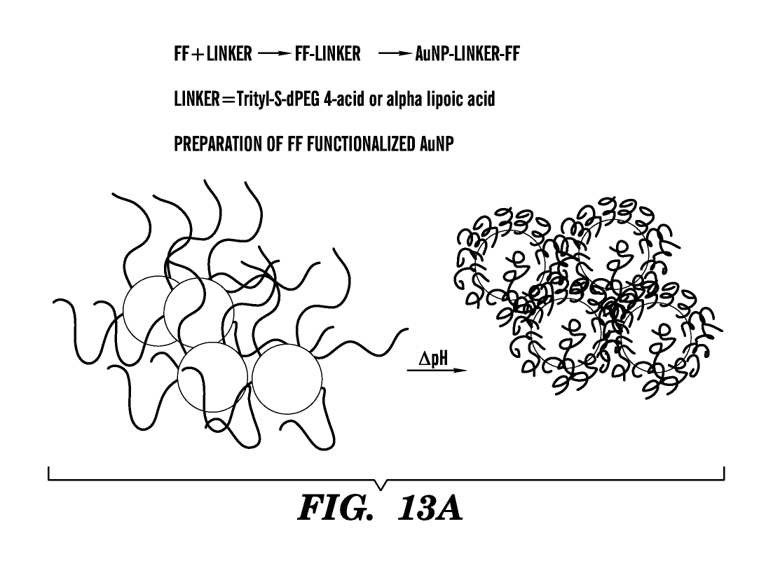

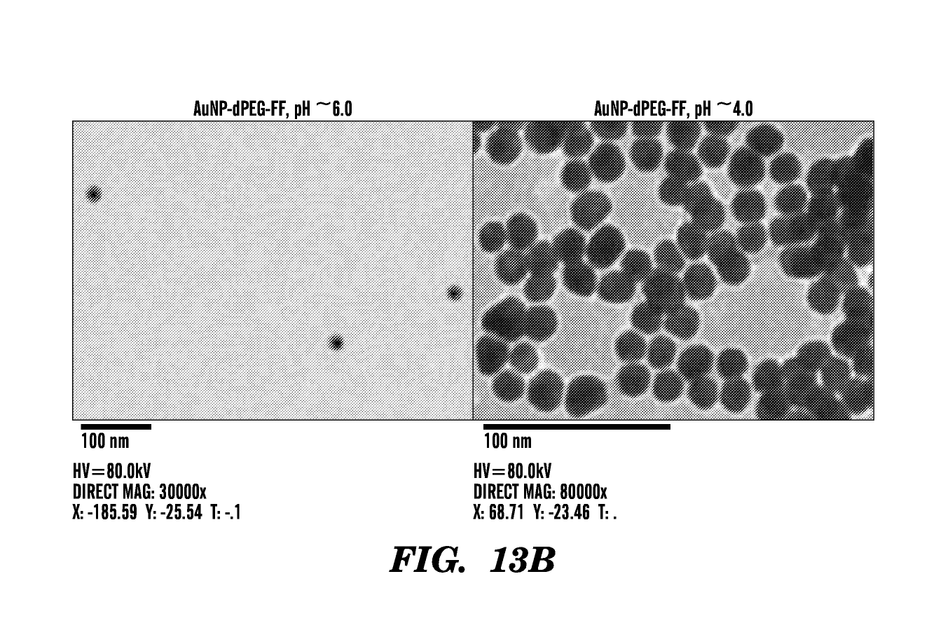

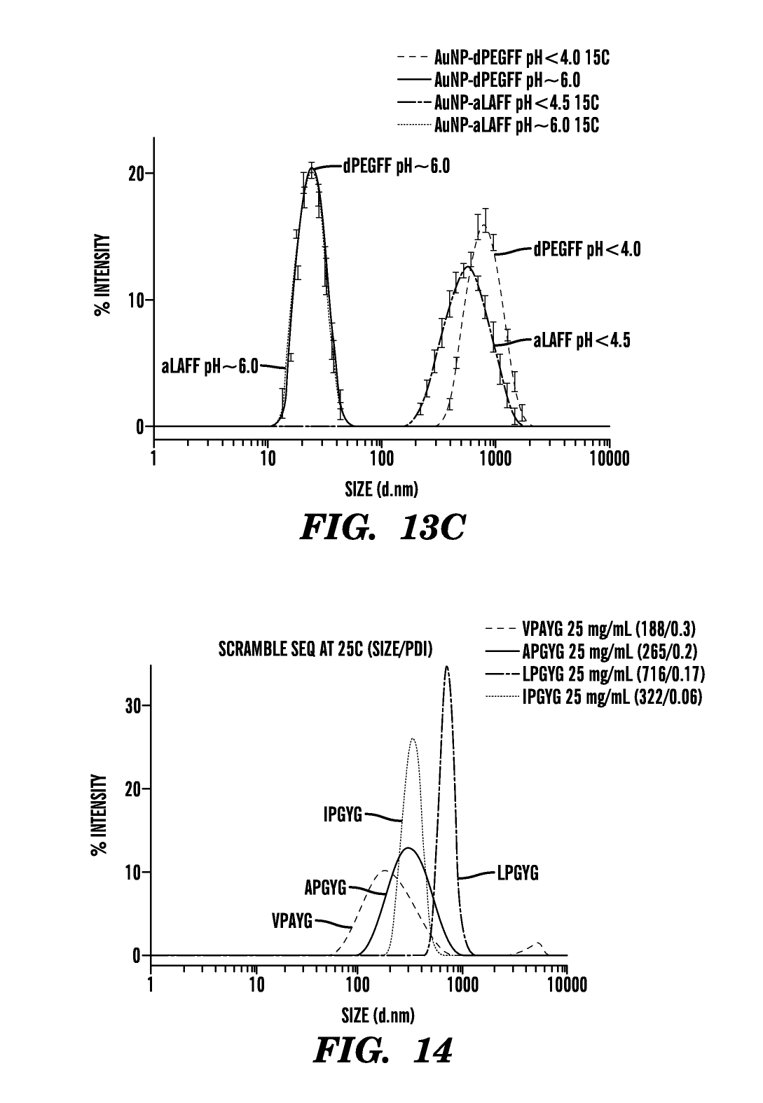

In some embodiments where at least one or a plurality of (e.g., 2 or more) isolated peptides are conjugated to a particle (e.g., but not limited to a nanoparticle), the peptide-conjugated particles can aggregate in response to a stimulus described herein, e.g., but not limited to pH change, or temperature change. For example, FIGS. 13A-13C show that nanoparticles functionalized with a plurality of the isolated peptides described herein (e.g., FF peptides shown in FIG. 1) can form a larger aggregate at a lower pH than at a higher pH. The formed aggregate can have a defined shape, e.g., a particle, a fiber, a rod, a tube, a vesicle, a ring, a prism, or any combinations thereof. Alternatively, the formed aggregate comprising the peptide-conjugated particles can form a random network, e.g., as shown in FIG. 13B.

Applications and Uses of the Isolated Peptides and/or Peptide Nanostructures

The isolated peptides and/or self-assembled peptide nanostructures can be formulated in different compositions and/or used in various applications. When used alone or when integrated into larger three-dimensional (3D) porous scaffolds, these nanomaterials can modulate the mechanical property of the local environment to alter tissue mechanics (e.g., in fibrosis or cancer), deliver a wide range of small molecules or active agents from small molecule drugs to biologics for therapeutic, diagnostic or imaging applications, regulate cellular activities (e.g., mechanically control stem cell fate switching, chemically inhibit enzyme activities), using a range of external stimuli or triggers (e.g., temperature, pH, etc.).

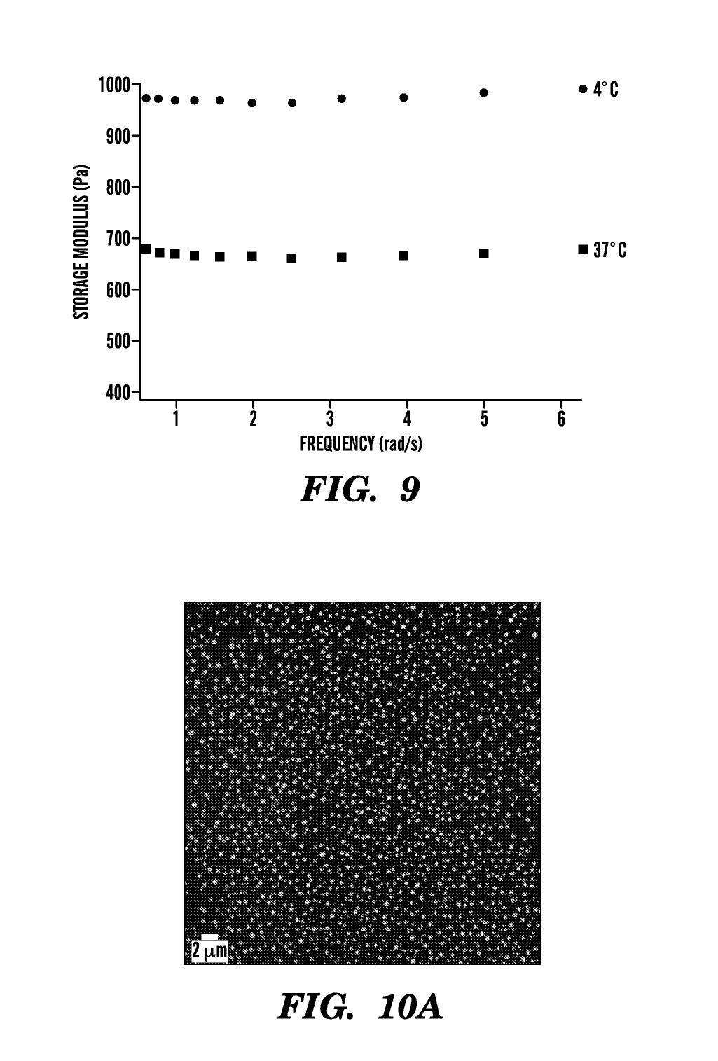

In some embodiments, the isolated peptides can be conjugated to a protein (e.g., an extracellular matrix protein) or a biopolymer to induce stimuli-dependent (e.g., temperature-dependent) gel formation. In other embodiments, the self-assembled nanostructure can be pre-formed from the peptide constructs described herein and then dispersed in a gel, a hydrogel, or a polymer, to induce stimuli-dependent (e.g., temperature-dependent) gel formation. For example, as shown in FIG. 9, the hydrogel stiffness can be modulated by temperatures through incorporation with peptide nanoparticles described herein. Thus, the peptide-incorporated gel, hydrogel, or polymer can be desirable for tissue engineering scaffolds to modulate its mechanical stiffness for each individual's need. In some embodiments, such peptide-incorporated gel, hydrogel, or polymer can also be used as a stimulus-sensitive drug delivery system. For example, the gel system can be incorporated with an active agent or a therapeutic agent, the release of which can be controlled by modulating the property of the gel (e.g., but not limited to pore size and/or porosity) with an external stimulus (e.g., temperature, and/or pH).

Accordingly, articles comprising at least one or more (e.g., 1, 2, 3, 4, 5, 6, 7, 8, 9, 10, 15, 20, 30, 40, 50, 60, 70, 80, 90, 100 or more) isolated peptides and/or self-assembled peptide nanostructures are also provided herein. Exemplary articles provided herein include, but are not limited to, a tissue engineered scaffold, a gel, a medication (e.g., but not limited to, a therapeutic agent, and a preventative agent) in any pharmaceutical composition described herein, a diagnostic agent (including, e.g., but not limited to, an imaging agent), a coating of a medical device, a delivery device or vehicle, a fabric, and any combinations thereof.

Yet another aspect described herein relates to compositions each comprising an isolated peptide described herein and/or a self-assembled peptide nanostructure described herein. In some embodiments, the isolated peptide can be present in a first amount sufficient to alter at least one property of the composition. In some embodiments, the self-assembled peptide nanostructure is present in a second amount sufficient to alter at least one property of the composition. For example, the first amount of the isolated peptides or the second amount of the peptide nanostructures used in the composition can be about 0.001 wt % to 99.9 wt %, depending on types or nature of the composition, and/or intended function of the isolated peptides and/or self-assembled peptide nanostructures in the composition. By way of example only, the isolated peptides and/or self-assembled peptide nanostructures can be used as a food additive in a food composition. In this embodiment, the first amount of the isolated peptide or the second amount of the nanostructures used in the food composition can range from about 0.001 wt % to about 50 wt %, from about 0.01% to about 25 wt %, or from about 0.05% to about 10 wt %.

Examples of properties of the composition that can be altered in the presence of the isolated peptide(s) and/or peptide nanostructure(s) can include, without limitations, consistency, stability, absorption, nutrient value, therapeutic potential, esthetics, flavor, olfactory property, material property, bioavailability, and any combinations thereof.

The compositions described herein can be formulated to suit the need for various applications. In some embodiments, the composition can be formulated to be a pharmaceutical composition described herein. Additional information about pharmaceutical compositions comprising the isolated peptides and/or peptide nanostructures described herein is described in detail later in the section “Pharmaceutical Compositions.”

In some embodiments, the composition can be formulated to be a personal care composition. For example, in some embodiments, the personal care composition can be formulated to be a hair care composition or a skin care composition in a form of a cream, oil, lotion, powder, serum, gel, shampoo, conditioner, ointment, foam, spray, aerosol, mousse, or any combinations thereof. In other embodiments, the composition can be formulated to be a cosmetic composition in a form of powder, lotion, cream, lipstick, nail varnish, hair dye, balm, spray, mascara, fragrance, solid perfume, or any combinations thereof. Additional information about personal care compositions comprising the isolated peptides and/or peptide nanostructures described herein is described in detail later in the section “Personal Care Compositions.”

In some embodiments, the composition can be formulated to be a food composition, including, but not limited to, solid food, liquid food, drinks, emulsions, slurries, curds, dried food products, packaged food products, raw food, processed food, powder, granules, dietary supplements, edible substances/materials, chewing gums, or any combinations thereof. The food compositions can include, but are not limited to, food compositions consumed by any subject, including, e.g., a human, or a domestic or game animal such as feline species, e.g., cat; canine species, e.g., dog; fox; wolf; avian species, e.g., chicken, emu, ostrich, birds; and fish, e.g., trout, catfish, salmon and pet fish.

In some embodiments, the isolated peptides and/or peptide nanostructures can be used to stabilize and/or provide a controlled release or a sustained release of at least one food ingredient, flavoring, nutrient, and/or vitamin.

In some embodiments, the isolated peptides and/or the peptide nanostructures can be used as a food additive in the food composition. Accordingly, a food additive comprising an isolated peptide and/or a peptide nanostructure is also described herein. In some embodiments of this aspect described herein, the isolated peptide and/or the peptide nanostructure can be configured to be capable of altering at least one property of a food composition upon addition of the isolated peptide and/or the peptide nanostructure to the food composition. For example, the composition and/or structure of the peptides (e.g., but not limited to, amino acid residues and/or length of the peptides described herein as well as the entities to which the peptides are conjugated to) can be configured such that the peptide(s) can alter at least one property of the food composition. Alternatively or additionally, the composition and/or structures of the peptide nanostructures (e.g., the amino acid residues and/or length of the self-assembling peptides, the entities to which the self-assembling peptides, as well as size, shape, porosity, and/or pore size of the peptide nanostructures) can be configured such that the peptide(s) can alter at least one property of the food composition.

The food additive can be present in any form, e.g., powder, particles, slurry, liquid, solution, solid, emulsion, colloid or any combinations thereof.

Accordingly, methods for altering at least one property of food or a food composition are also provided herein. For example, some embodiments of the methods described herein can be used to alter consistency, stability, absorption, nutrient value, esthetics, flavor, olfactory property, material property, or any combinations thereof, of the food or food composition. The method comprises providing food or a food composition described herein, which comprises an effective amount of the isolated peptides and/or the peptide nanostructures described herein, wherein the effective amount is sufficient to alter at least one property of the food or the food composition.

In some embodiments, at least a portion of the isolated peptides and/or the peptide nanostructures in the food or food composition can be capable of responding to at least one stimulus. Examples of a stimulus can include, without limitations, of a change in light intensity and/or wavelength, a change in pH, a change in temperature, a change in humidity, and any combinations thereof. In these embodiments, the method can further comprise exposing the isolated peptides and/or the peptide nanostructures to said at least one stimulus, wherein the response of the isolated peptides and/or the peptide nanostructures to said at least one stimulus alters said at least one property of the food or the food composition. In some embodiments, the response of the isolated peptides can include, but are not limited to, a conformational change, a change in interaction between the isolated peptides within the food or food composition, a change in interaction between the isolated peptides and at least one component of the food or food composition, size and/or shape of the peptide nanostructures formed from the isolated peptides, or a combinations thereof. In some embodiments, the response of the peptide nanostructures can include, but are not limited to, a change in size, shape, pore size, and/or porosity of the nanostructures within the food or food composition, a change in interaction between the peptide nanostructures and at least one component of the food or food composition, and any combinations thereof.

In some embodiments, the method can further comprise contacting the food or the food composition with the effective amount of the isolated peptides and/or the peptide nanostructures described herein.

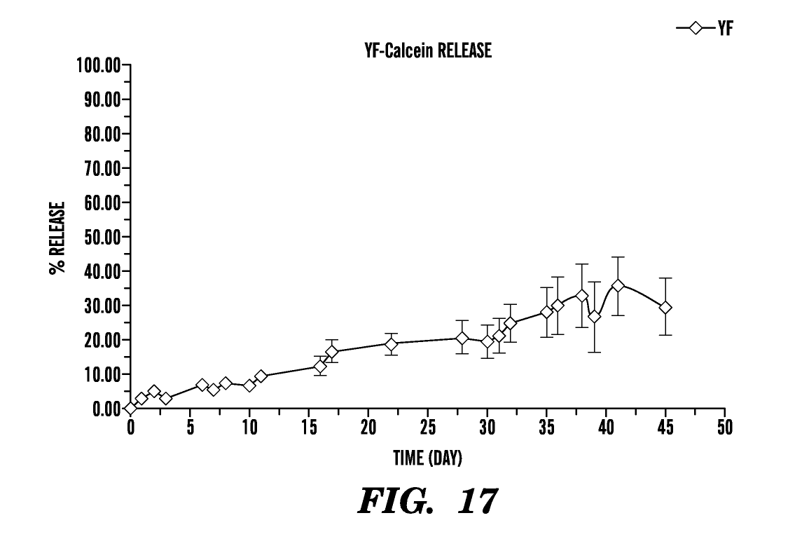

In some embodiments, an active agent can be conjugated to an isolated peptide described herein and/or encapsulated in the peptide nanostructure described herein to control the release of the active agent (e.g., as shown in FIG. 17). Thus, in another aspect, a method of modulating release of an active agent from a composition or an article is provided herein. For example, an active agent can be controllably released from a composition or an article described herein over a period of time, e.g., at least 1 hour, at least about 6 hours, at least about 12 hours, at least about 1 day, at least about 2 days, at least about 3 days, at least about 4 days, at least about 5 days, at least about 6 days, at least about 7 days, at least about 2 weeks, at least about 3 weeks, at least about 4 weeks, at least about 2 months, at least about 3 months, at least about 4 months, at least about 5 months, at least about 6 months, at least about 9 months, at least about 1 year or longer. The method comprises (a) providing a composition or an article comprising an active agent distributed in at least one or more (e.g., 1, 2, 3, 4, 5, 6, 7, 8, 9, 10, 15, 20, 30, 40, 50, 60, 70, 80, 90, 100 or more) peptide nanostructures described herein, wherein at least a portion of the peptide nanostructures are capable of responding to at least one stimulus; and (b) exposing to said at least one stimulus the peptide nanostructures within the composition or the article. The response of the peptide nanostructures to said at least one stimulus modulates the release of the active agent from the nanostructures. Examples of the stimulus can include, without limitations, a change in light intensity and/or wavelength, a change in pH, a change in temperature, a change in humidity, and any combinations thereof.

In some embodiments, the response of the peptide nanostructures to the stimulus can include a change in size, pore size and/or porosity of the peptide nanostructures. Thus, by changing the size, pore size and/or porosity of the peptide nanostructures, the amount and/or rate of the active agent released from the peptide nanostructures can be controlled.

The peptide nanostructures used in the composition or article can be of any form. For example, the peptide nanostructures can be in a form of a particle, a rod, a prism, a disc, a fiber, a vesicle, a ring, an aggregate (e.g., no defined shape) or any combinations thereof. In one embodiment, peptide nanoparticles are used in the composition.

The composition or article can be any composition used to deliver an active agent, e.g., but not limited to, a pharmaceutical composition described herein, a cosmetic composition (e.g., a composition for treatment of skin and/or hair, or for use in cosmetic or aesthetic surgery), a nutraceutical composition (e.g., but not limited to, fortified food and/or dietary supplements), an injectable composition (e.g., a composition that can be administered by injection), a patch, a bandage, a scaffold, a coating, or any combinations thereof. In some embodiments, the composition or article can be in a form of a gel, a scaffold, a film, a patch, a particle, a cream, a lotion, an ointment, a solution, a capsule, a pill, a tablet, powder, a paste, or any combinations thereof.

A further aspect provided herein relates to a method of modulating at least one material property and/or structure of a matrix, e.g., but not limited to, a scaffold, a gel, a tissue, or a cell. The method comprises (a) providing a matrix comprising a plurality of (e.g., 2 or more) the isolated peptides and/or the peptide nanostructures described herein, wherein at least a portion of the isolated peptides and/or the peptide nanostructures are capable of responding to at least one stimulus; and (b) exposing to said at least one stimulus the isolated peptides and/or the peptide nanostructures within the matrix. The response of the isolated peptides and/or the peptide nanostructure to said at least one stimulus modulates at least one material property of the matrix. Examples of the stimulus can include, without limitations, a change in light intensity and/or wavelength, a change in pH, a change in temperature, a change in humidity, and any combinations thereof.

Examples of material properties of a matrix that can be modulated using the method described herein can include, but are not limited to, chemical properties (e.g., but not limited to, pH, reactivity, surface tension, hydrophobicity); electrical properties (e.g., conductivity); magnetic properties; mechanical properties (e.g., but not limited to, compressive strength, ductility, fatigue limit, hardness, plasticity, shear strength, tensile strength, stiffness, yield strength, Young's modulus, viscoelasticity); optical properties (e.g., but not limited to absorptivity, color, photosensitivity, scattering); thermal properties (e.g., but not limited to, glass transition temperature, thermal conductivity, melting point, thermal expansion); physical property (e.g., but not limited to density, porosity, pore size, solubility) or any combinations thereof.

In some embodiments, the methods described herein can be used to modulate at least one material property of the matrix selected from the group consisting of viscosity, porosity, mechanical stiffness, ductility, viscoelasticity, organization, degradability, solubility, density, flexibility, permeability, hydrophobicity, optical properties, thermal properties, and any combinations thereof.

By way of example only, in some embodiments, the isolated peptides distributed in the matrix can be conjugated to an optical labeling agent (e.g., a fluorescent molecule, a quantum dot) and/or the peptide nanostructures distributed in the matrix can be loaded with an optical labeling agent, thereby modulating an optical property of the matrix. In some embodiments, the amino acid sequence of the isolated peptides distributed in the matrix can affect the optical property of the matrix, as without wishing to be bound by theory, amino acid residues can absorb or emit electromagnetic energy at different wavelengths.

As another example, as shown in FIG. 9 described earlier, the hydrogel stiffness can be modulated by temperatures through incorporation with peptide nanoparticles described herein and/or isolated peptides described herein. In some embodiments, the peptide nanoparticles and/or the isolated peptides can be conjugated to hydrogel-forming precursors or residues.

In some embodiments, the response of the isolated peptides within the matrix can include a conformational change, a change in interaction between the isolated peptides within the matrix, a change in interaction between the isolated peptides and at least one component of the matrix, size and/or shape of the peptide nanostructures formed from the isolated peptides, or any combinations thereof.

In some embodiments, the response of the peptide nanostructures within the matrix can include a change in size, pore size, and/or porosity of the nano structures within the matrix.

The peptide nanostructures used in the composition or article can be of any form. For example, the peptide nanostructures can be in a form of a particle, a rod, a prism, a disc, a fiber, or any combinations thereof. In one embodiment, peptide nanoparticles are used in the composition or article.

In some embodiments, the method can further comprise introducing the isolated peptides and/or the peptide nanostructures into the matrix. For example, in some embodiments, the isolated peptides and/or the peptide nanostructures can be introduced into a solution or suspension prior to formation of a scaffold or a gel. In other embodiments, the isolated peptides and/or the peptide nanostructures can be introduced into a cell or at least a portion of a tissue by injection or microinjection. In some embodiments, the isolated peptides and/or the peptide nanostructures can comprise a cell surface receptor-targeting ligand, which can facilitate the uptake of the isolated peptides and/or the peptide nanostructures by at least one cell or a cell present in the tissue and/or promote targeted delivery to specific cells or specific cells present in the tissue.

In some embodiments, the method can be used to modulate the mechanical stiffness of at least a portion of a tissue in a subject, e.g., a mammalian subject such as a human being. In these embodiments, the isolated peptides and/or the peptide nanostructures described herein can be injected to a target site in a tissue in vivo.

Another aspect provided herein relates to a method of inducing gel formation of a protein or polymer. The method comprises (a) providing a solution or suspension of a protein or polymer, wherein at least a portion of the protein or polymer molecules are conjugated to the isolated peptides described herein, and wherein the isolated peptides are capable of responding to at least one stimulus; and (b) exposing to said at least one stimulus the isolated peptide within the solution or suspension. The response of the isolated peptides conjugated to the protein or polymer molecules induces aggregation of the protein or polymer molecules to form a gel. Examples of the stimulus can include, without limitations, a change in light intensity and/or wavelength, a change in pH, a change in temperature, a change in humidity, and any combinations thereof.

Yet another aspect provided herein relates to a method of modulating at least one behavior of a biological cell, e.g., but not limited to, growth, viability, migration, differentiation, secretion, protein synthesis, apoptosis, fate switching, contractibility, or any combinations thereof. The method comprises contacting a biological cell with one or more embodiments of a composition described herein. In some embodiments, the isolated peptide(s) and/or the peptide nanostructure(s) within the composition can be configured to be bioactive (e.g., being capable of modulating at least one behavior of a biological cell) even without any added bioactive agent. In some embodiments, the isolated peptide(s) and/or the peptide nanostructure(s) within the composition can be configured to be inert. In these embodiments, the isolated peptide(s) can be conjugated to a bioactive agent, and/or the peptide nanostructures can encapsulate a bioactive agent.

The method described herein can be performed in vitro or in vivo. In some embodiments, the biological cell can be present in vitro. In other embodiments, the biological cell can be present in a subject, e.g., a mammalian subject. In these embodiments, the biological cell in the subject can be contacted with the composition by administering the subject with the composition in any appropriate manner, e.g., oral administration and/or parenteral administration, depending on the formulation of the composition. In some embodiments, the composition can be a pharmaceutical composition, a food composition or a personal care composition described herein.

### Kits

Kits comprising the isolated peptides and/or self-assembled peptide nanostructures are also provided herein. In some embodiments, a plurality of the isolated peptides and/or self-assembled peptide nanostructures can be provided in a kit, which further comprises at least one reagent. The reagent can also include a coupling molecule or agent for linking an isolated peptide and/or peptide nanostructure to a substrate as described herein. In some embodiments, the kit can further comprise an active agent.

In addition to the above mentioned components, the kit can include informational material. The informational material can be descriptive, instructional, marketing or other material that relates to the methods described herein and/or the use/storage of the self-assembled nanostructures. For example, the informational material describes methods to form peptide nanostructures using the isolated peptides described herein; and/or methods for administering the peptide nanostructures to a subject; and/or methods to use the isolated peptides and/or peptide nanostructures, e.g., for increasing the mechanical stiffness of a matrix and/or inducing gel formation of a protein or polymer as described earlier. The kit can also include a delivery device.

In one embodiment, the informational material can include instructions to administer the formulation in a suitable manner, e.g., in a suitable dose, dosage form, or mode of administration (e.g., a dose, dosage form, or mode of administration described herein). In another embodiment, the informational material can include instructions for identifying a suitable subject, e.g., a human. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the formulation and/or its use in the methods described herein. Of course, the informational material can also be provided in any combination of formats.

In some embodiments the individual components of the formulation can be provided in one container. Alternatively, it can be desirable to provide the components of the formulation separately in two or more containers, e.g., one container for a self-assembling peptide preparation, and at least another for a carrier compound. The different components can be combined, e.g., according to instructions provided with the kit. The components can be combined according to a method described herein, e.g., to prepare and administer a pharmaceutical composition.

In addition to the formulation, the composition of the kit can include other ingredients, such as a solvent or buffer, a stabilizer or a preservative, and/or a second agent for treating a condition or disorder described herein. Alternatively, the other ingredients can be included in the kit, but in different compositions or containers than the formulation. In such embodiments, the kit can include instructions for admixing the formulation and the other ingredients, or for using the oligonucleotide together with the other ingredients.

The formulation can be provided in any form, e.g., liquid, dried or lyophilized form. It is preferred that the formulation be substantially pure and/or sterile. When the formulation is provided in a liquid solution, the liquid solution preferably is an aqueous solution, with a sterile aqueous solution being preferred. When the formulation is provided as a dried form, reconstitution generally is by the addition of a suitable solvent. The solvent, e.g., sterile water or buffer, can optionally be provided in the kit.

In some embodiments, the kit contains separate containers, dividers or compartments for the formulation and informational material. For example, the formulation can be contained in a bottle, vial, or syringe, and the informational material can be contained in a plastic sleeve or packet. In other embodiments, the separate elements of the kit are contained within a single, undivided container. For example, the formulation is contained in a bottle, vial or syringe that has attached thereto the informational material in the form of a label.

In some embodiments, the kit includes a plurality, e.g., a pack, of individual containers, each containing one or more unit dosage forms of the formulation. For example, the kit includes a plurality of syringes, ampules, foil packets, or blister packs, each containing a single unit dose of the formulation. The containers of the kits can be air tight and/or waterproof.

### Amino Acid Residue and Exemplary Derivatives Thereof

As used herein, the term “amino acid residue” includes amino acid selected from the group consisting of alanine; arginine; asparagine; aspartic acid; cysteine; glutamic acid; glutamine; glycine; histidine; isoleucine; leucine; lysine; methionine; phenylalanine; proline; serine; threonine; tryptophan; tyrosine; valine; homocysteine; phosphoserine; phosphothreonine; phosphotyrosine; hydroxyproline; γ-carboxyglutamate; hippuric acid; octahydroindole-2-carboxylic acid; statine; 1,2,3,4,-tetrahydroisoquinoline-3-carboxylic acid; penicillamine (3-mercapto-D-valine); ornithine (Orn); citruline; alpha-methyl-alanine; para-benzoylphenylalanine; para-aminophenylalanine; p-fluorophenylalanine; phenylglycine; propargylglycine; N-methylglycins (sarcosine, Sar); and tert-butylglycine; diaminobutyric acid; 7-hydroxy-tetrahydroisoquinoline carboxylic acid; naphthylalanine; biphenylalanine; cyclohexylalanine; amino-isobutyric acid (Aib); norvaline; norleucine (Nle); tert-leucine; tetrahydroisoquinoline carboxylic acid; pipecolic acid; phenylglycine; homophenylalanine; cyclohexylglycine; dehydroleucine; 2,2-diethylglycine; 1-amino-1-cyclopentanecarboxylic acid; 1-amino-1-cyclohexanecarboxylic acid; amino-benzoic acid; amino-naphthoic acid; gamma-aminobutyric acid; difluorophenylalanine; nipecotic acid; N-α-imidazole acetic acid (IMA); thienyl-alanine; t-butylglycine; desamino-Tyr; aminovaleric acid (Ava); pyroglutaminic acid (<Glu); α-aminoisobutyric acid (αAib); γ-aminobutyric acid (γAbu); α-aminobutyric acid (αAbu); αγ-aminobutyric acid (αγAbu); 3-pyridylalanine (Pal); Isopropyl-α-N^{ε}lysine (ILys); Napthyalanine (Nal); α-napthyalanine (α-Nal); β-napthyalanine (β-Nal); Acetyl-β-napthyalanine (Ac-β-napthyalanine); α,β-napthyalanine; N^{ε}-picoloyl-lysine (PicLys); 4-halo-Phenyl; 4-pyrolidylalanine; isonipecotic carboxylic acid (inip); beta-amino acids; and isomers, analogs and derivatives thereof. One of skill in the art would know that this definition includes, D- and L-amino acids; alpha-, beta- and gamma-amino acids; chemically modified amino acids; naturally occurring non-proteogenic amino acids; rare amino acids; and chemically synthesized compounds that have properties known in the art to be characteristic of an amino acid. Additionally, each embodiment can include any combinations of the groups.

Furthermore, as used herein, the term “amino acid” includes compounds which depart from the structure of the naturally occurring amino acids, but which have substantially the structure of an amino acid, such that they can be substituted within a peptide which retains is activity, e.g., aggregate forming activity. Thus, for example, in some embodiments amino acids can also include amino acids having side chain modifications or substitutions, and also include related organic acids, amides or the like. Without limitation, an amino acid can be a proteogenic or non-proteogenic amino acid.

In some embodiments, an amino acid residue can include a chemically modified amino acid. As used herein, the term “chemically modified amino acid” refers to an amino acid that has been treated with one or more reagents.

In some embodiments, an amino acid residue can include a beta-amino acid. Exemplary beta-amino acids include, but are not limited to, L-β-Homoproline hydrochloride; (±)-3-(Boc-amino)-4-(4-biphenylyl)butyric acid; (±)-3-(Fmoc-amino)-2-phenylpropionic acid; (1S,3R)-(+)-3-(Boc-amino)cyclopentanecarboxylic acid; (2R,3R)-3-(Boc-amino)-2-hydroxy-4-phenylbutyric acid; (2S,3R)-3-(Boc-amino)-2-hydroxy-4-phenylbutyric acid; (R)-2-[(Boc-amino)methyl]-3-phenylpropionic acid; (R)-3-(Boc-amino)-2-methylpropionic acid; (R)-3-(Boc-amino)-2-phenylpropionic acid; (R)-3-(Boc-amino)-4-(2-naphthyl)butyric acid; (R)-3-(Boc-amino)-5-phenylpentanoic acid; (R)-3-(Fmoc-amino)-4-(2-naphthyl)butyric acid; (R)-(−)-Pyrrolidine-3-carboxylic acid; (R)-Boc-3,4-dimethoxy-β-Phe-OH; (R)-Boc-3-(3-pyridyl)-β-Ala-OH; (R)-Boc-3-(trifluoromethyl)-β-Phe-OH; (R)-Boc-3-cyano-β-Phe-OH; (R)-Boc-3-methoxy-β-Phe-OH; (R)-Boc-3-methyl-β-Phe-OH; (R)-Boc-4-(4-pyridyl)-β-Homoala-OH; (R)-Boc-4-(trifluoromethyl)-β-Homophe-OH; (R)-Boc-4-(trifluoromethyl)-β-Phe-OH; (R)-Boc-4-bromo-β-Phe-OH; (R)-Boc-4-chloro-β-Homophe-OH; (R)-Boc-4-chloro-β-Phe-OH; (R)-Boc-4-cyano-β-Homophe-OH; (R)-Boc-4-cyano-β-Phe-OH; (R)-Boc-4-fluoro-β-Phe-OH; (R)-Boc-4-methoxy-β-Phe-OH; (R)-Boc-4-methyl-β-Phe-OH; (R)-Boc-β-Tyr-OH; (R)-Fmoc-4-(3-pyridyl)-β-Homoala-OH; (R)-Fmoc-4-fluoro-β-Homophe-OH; (S)-(+)-Pyrrolidine-3-carboxylic acid; (S)-3-(Boc-amino)-2-methylpropionic acid; (S)-3-(Boc-amino)-4-(2-naphthyl)butyric acid; (S)-3-(Boc-amino)-5-phenylpentanoic acid; (S)-3-(Fmoc-amino)-2-methylpropionic acid; (S)-3-(Fmoc-amino)-4-(2-naphthyl)butyric acid; (S)-3-(Fmoc-amino)-5-hexenoic acid; (S)-3-(Fmoc-amino)-5-phenyl-pentanoic acid; (S)-3-(Fmoc-amino)-6-phenyl-5-hexenoic acid; (S)-Boc-2-(trifluoromethyl)-β-Homophe-OH; (S)-Boc-2-(trifluoromethyl)-β-Homophe-OH; (S)-Boc-2-(trifluoromethyl)-β-Phe-OH; (S)-Boc-2-cyano-β-Homophe-OH; (S)-Boc-2-methyl-β-Phe-OH; (S)-Boc-3,4-dimethoxy-β-Phe-OH; (S)-Boc-3-(trifluoromethyl)-β-Homophe-OH; (S)-Boc-3-(trifluoromethyl)-β-Phe-OH; (S)-Boc-3-methoxy-β-Phe-OH; (S)-Boc-3-methyl-β-Phe-OH; (S)-Boc-4-(4-pyridyl)-β-Homoala-OH; (S)-Boc-4-(trifluoromethyl)-β-Phe-OH; (S)-Boc-4-bromo-β-Phe-OH; (S)-Boc-4-chloro-β-Homophe-OH; (S)-Boc-4-chloro-β-Phe-OH; (S)-Boc-4-cyano-β-Homophe-OH; (S)-Boc-4-cyano-β-Phe-OH; (S)-Boc-4-fluoro-β-Phe-OH; (S)-Boc-4-iodo-β-Homophe-OH; (S)-Boc-4-methyl-β-Homophe-OH; (S)-Boc-4-methyl-β-Phe-OH; (S)-Boc-β-Tyr-OH; (S)-Boc-γ,γ-diphenyl-β-Homoala-OH; (S)-Fmoc-2-methyl-β-Homophe-OH; (S)-Fmoc-3,4-difluoro-β-Homophe-OH; (S)-Fmoc-3-(trifluoromethyl)-β-Homophe-OH; (S)-Fmoc-3-cyano-β-Homophe-OH; (S)-Fmoc-3-methyl-β-Homophe-OH; (S)-Fmoc-γ,γ-diphenyl-β-Homoala-OH; 2-(Boc-aminomethyl)phenylacetic acid; 3-Amino-3-(3-bromophenyl)propionic acid; 3-Amino-4,4,4-trifluorobutyric acid; 3-Aminobutanoic acid; DL-3-Aminoisobutyric acid; DL-β-Aminoisobutyric acid puriss; DL-β-Homoleucine; DL-β-Homomethionine; DL-β-Homophenylalanine; DL-β-Leucine; DL-β-Phenylalanine; L-β-Homoalanine hydrochloride; L-β-Homoglutamic acid hydrochloride; L-β-Homoglutamine hydrochloride; L-β-Homohydroxyproline hydrochloride; L-β-Homoisoleucine hydrochloride; L-β-Homoleucine hydrochloride; L-β-Homolysine dihydrochloride; L-β-Homomethionine hydrochloride; L-β-Homophenylalanine allyl ester hydrochloride; L-β-Homophenylalanine hydrochloride; L-β-Homoserine; L-β-Homothreonine; L-β-Homotryptophan hydrochloride; L-β-Homotyrosine hydrochloride; L-β-Leucine hydrochloride; Boc-D-β-Leu-OH; Boc-D-β-Phe-OH; Boc-β³-Homopro-OH; Boc-β-Glu(OBzl)-OH; Boc-β-Homoarg(Tos)-OH; Boc-β-Homoglu(OBzl)-OH; Boc-β-Homohyp(Bzl)-OH (dicyclohexylammonium) salt technical; Boc-β-Homolys(Z)-OH; Boc-β-Homoser(Bzl)-OH; Boc-β-Homothr(Bzl)-OH; Boc-β-Homotyr(Bzl)-OH; Boc-β-Ala-OH; Boc-β-Gln-OH; Boc-β-Homoala-OAll; Boc-β-Homoala-OH; Boc-β-Homogln-OH; Boc-β-Homoile-OH; Boc-β-Homoleu-OH; Boc-β-Homomet-OH; Boc-β-Homophe-OH; Boc-β-Homotrp-OH; Boc-β-Homotrp-OMe; Boc-β-Leu-OH; Boc-β-Lys(Z)-OH (dicyclohexylammonium) salt; Boc-β-Phe-OH; Ethyl 3-(benzylamino)propionate; Fmoc-D-β-Homophe-OH; Fmoc-L-β³-homoproline; Fmoc-β-D-Phe-OH; Fmoc-β-Gln(Trt)-OH; Fmoc-β-Glu(OtBu)-OH; Fmoc-β-Homoarg(Pmc)-OH; Fmoc-β-Homogln(Trt)-OH; Fmoc-β-Homoglu(OtBu)-OH; Fmoc-β-Homohyp(tBu)-OH; Fmoc-β-Homolys(Boc)-OH; Fmoc-β-Homoser(tBu)-OH; Fmoc-β-Homothr(tBu)-OH; Fmoc-β-Homotyr(tBu)-OH; Fmoc-β-Ala-OH; Fmoc-β-Gln-OH; Fmoc-β-Homoala-OH; Fmoc-β-Homogln-OH; Fmoc-β-Homoile-OH; Fmoc-β-Homoleu-OH; Fmoc-β-Homomet-OH; Fmoc-β-Homophe-OH; Fmoc-β-Homotrp-OH; Fmoc-β-Leu-OH; Fmoc-β-Phe-OH; N-Acetyl-DL-β-phenylalanine; Z-D-β-Dab(Boc)-OH; Z-D-β-Dab(Fmoc)-OH purum; Z-DL-β-Homoalanine; Z-β-D-Homoala-OH; Z-β-Glu(OtBu)-OH technical; Z-β-Homotrp(Boc)-OH; Z-β-Ala-OH purum; Z-β-Ala-ONp purum; Z-β-Dab(Boc)-OH; Z-β-Dab(Fmoc)-OH; Z-β-Homoala-OH; β-Alanine; β-Alanine BioXtra; β-Alanine ethyl ester hydrochloride; β-Alanine methyl ester hydrochloride; β-Glutamic acid hydrochloride; cis-2-Amino-3-cyclopentene-1-carboxylic acid hydrochloride; cis-3-(Boc-amino)cyclohexanecarboxylic acid; and cis-3-(Fmoc-amino)cyclohexanecarboxylic acid.

### Self-Assembling Peptide Synthesis

The self-assembling peptides described herein can be synthesized according to art-recognized methods of solution and solid phase peptide chemistry, or by classical methods known in the art. Cleavage of synthesized peptides from a resin and purification of peptides are well known in the art. Cleavage of synthesized peptides from a resin can be done, for example, in a solution containing trifluoroacetic acid. Purification of synthesized peptides can be done, for example, by chromatography such as HPLC. Methods describing useful peptide synthesis and purification methods can be found, for example, in U.S. Pat. App. Pub. No. 20060084607, content of which is incorporated herein by reference, as well as the methods described in the Examples.

Peptides described herein can be synthetically constructed by suitable known peptide polymerization techniques, such as exclusively solid phase techniques, partial solid-phase techniques, fragment condensation or classical solution couplings. For example, the peptides of the invention can be synthesized by the solid phase method using standard methods based on either t-butyloxycarbonyl (BOC) or 9-fluorenylmethoxy-carbonyl (FMOC) protecting groups. This methodology is described by G. B. Fields et al. in Synthetic Peptides: A User's Guide, W. M. Freeman & Company, New York, N.Y., pp. 77-183 (1992) and in the textbook “Solid-Phase Synthesis”, Stewart & Young, Freemen & Company, San Francisco, 1969, and are exemplified by the disclosure of U.S. Pat. No. 4,105,603, issued Aug. 8, 1979. Classical solution synthesis is described in detail in “Methoden der Organischen Chemic (Houben-Weyl): Synthese von Peptiden”, E. Wunsch (editor) (1974) Georg Thieme Verlag, Stuttgart West Germany. The fragment condensation method of synthesis is exemplified in U.S. Pat. No. 3,972,859. Other available syntheses are exemplified in U.S. Pat. No. 3,842,067, U.S. Pat. No. 3,872,925, issued Jan. 28, 1975, Merrifield B, Protein Science (1996), 5: 1947-1951; The chemical synthesis of proteins; Mutter M, Int J Pept Protein Res 1979 March; 13 (3): 274-7 Studies on the coupling rates in liquid-phase peptide synthesis using competition experiments; and Solid Phase Peptide Synthesis in the series Methods in Enzymology (Fields, G. B. (1997) Solid-Phase Peptide Synthesis. Academic Press, San Diego.#9830). Content of all of the foregoing disclosures is incorporated herein by reference.

In some embodiments, the self-assembling peptide can be a peptide mimetic. Methods of designing peptide mimetics and screening of functional peptide mimetics are well known to those skilled in the art. One basic method of designing a molecule which mimics a known protein or peptide is first to identify the active region(s) of the known protein (for example, in the case of an antibody-antigen interaction, one identifies which region(s) of the antibody that permit binding to the antigen), and then searches for a mimetic which emulates the active region. If the active region of a known protein is relatively small, it is anticipated that a mimetic will be smaller (e.g. in molecular weight) than the protein, and correspondingly easier and cheaper to synthesize. Such a mimetic could be used as a convenient substitute for the protein, as an agent for interacting with the target molecule.

Methods for preparing peptide mimetics include modifying the N-terminal amino group, the C-terminal carboxyl group, and/or changing one or more of the amide linkages in the peptide to a non-amide or a modified amide linkage. Two or more such modifications can be coupled in one peptide mimetic. Modifications of peptides to produce peptide mimetics are described, for example, in U.S. Pat. No. 5,643,873 and No. 5,654,276, content of both of which is incorporated herein by reference.

For example, Reineke et al. (1999, Nature Biotechnology, 17; 271-275, content of which is herein incorporated by reference) designed a mimic molecule which mimics a binding site of the interleukin-10 protein using a large library of short synthetic peptides, each of which corresponded to a short section of interleukin 10. The binding of each of these peptides to the target (in this case an antibody against interleukin-10) was then tested individually by an assay technique, to identify potentially relevant peptides. Phage display libraries of peptides and alanine scanning method can be used.

Other methods for designing peptide mimetics to a particular peptide or protein include those described in European Patent EP1206494, the SuperMimic program by Andrean Goede et. al. 2006 BMC Bioinformatics, 7:11; and MIMETIC program by W. Campbell et al., 2002, Microbiology and Immunology 46:211-215. The SuperMimic program is designed to identify compounds that mimic parts of a protein, or positions in proteins that are suitable for inserting mimetics. The application provides libraries that contain peptidomimetic building blocks on the one hand and protein structures on the other. The search for promising peptidomimetic linkers for a given peptide is based on the superposition of the peptide with several conformers of the mimetic. New synthetic elements or proteins can be imported and used for searching. The MIMETIC computer program, which generates a series of peptides for interaction with a target peptide sequence is taught by W. Campbell et. al., 2002. In depth discussion of the topic is reviewed in “Peptide Mimetic Design with the Aid of Computational Chemistry” by James R. Damewood Jr. in Reviews in Computational Chemistry Reviews in Computational Chemistry, January 2007, Volume 9 Book Series: Reviews in Computational Chemistry, Editor(s): Kenny B. Lipkowitz, Donald B. BoydPrint ISBN: 9780471186397 ISBN: 9780470125861 Published by John Wiley &Sons, Inc.; and in T. Tselios, et. al., Amino Acids, 14: 333-341, 1998. Content of all of the references described in this paragraph is herein incorporated by reference.

Methods for preparing libraries containing diverse populations of peptides, peptoids and peptidomimetics are well known in the art and various libraries are commercially available (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). One skilled in the art understands that a peptide can be produced in vitro directly or can be expressed from a nucleic acid, which can be produced in vitro. Methods of synthetic peptide and nucleic acid chemistry are well known in the art. Content of all of the references described in this paragraph is herein incorporated by reference.

### Assembly and Fabrication of Various Peptide Nanostructures

In accordance with one ospect described herein, simple, inexpensive, and scalable methods for generating peptide nanostructures (e.g., monodisperse, polydisperse, and/or stable nanostructures) using the short peptides are provided herein. In some embodiments, the peptide nanostructures can be formed in seconds from a mixture of the short peptides described herein. The peptide nanostructures can be tuned for a range of material property (e.g., but not limited to size, polydispersity or mondispersity, shape, porosity, pore size, mechanical stability and/or stability) by varying at least one parameter of a peptide self-assembly process, e.g., composition, temperature and/or pH of the formulation medium, the amino acid sequence and/or concentration of the peptides, and any combinations thereof.

As used herein, the term “formulation medium” refers to a medium in which self-assembly or self-organization of the peptides described herein occurs to form one or more embodiments of the peptide nanostructures. Thus, peptide nanostructures can be formed and dispersed in the formulation medium. In some embodiments, depending on fabrication methods, a formulation medium can be a medium in which one or more embodiments of the peptides described herein are dispersed or dissolved. In some embodiments, the formulation medium can comprise peptides having the same amino acid sequence. In other embodiments, the formulation medium can comprise peptides having different amino acid sequences.

For example, as shown in Example 2, self-assembly of peptide constructs can be induced by directly mixing self-assembling peptides in a formulation medium comprising an aqueous solvent (e.g., water, a salt solution and/or a buffered solution) at a certain temperature (e.g., from ˜2° C. to about room temperature). In some embodiments, a solvent injection protocol can be used for fabrication of self-assembled peptide nanostructures. In such embodiments, for example, as shown in Example 3, the self-assembling peptides can be first dissolved in an organic solvent (e.g., but not limited to, dimethyl sulfoxide (DMSO), acetone, ethanol, dioxane, acetonitrile, methanol, THF, or any combinations thereof) and then a fraction or a fixed volume of the dissolved peptides can be introduced (e.g., by injection) in a formulation medium comprising an aqueous solvent (e.g., water, a salt solution and/or a buffered solution). The fraction or the fixed volume of the dissolved peptides introduced into the formulation medium can depend on the scale of the final formulation. For example, in some embodiments, the ratio of the fixed volume to the volume of the formulation medium can be in a range of about 1:20 to about 1:1. Stated another way, the fraction or the fixed volume can be about 5% to about 50% of the final formulation volume.

The pH of the formulation medium (e.g., an aqueous solvent) can be acidic, neutral or basic. Different pHs of the aqueous solvent can lead to formation of nanostructures of different shape and/or size.

The formulation medium (e.g., an aqueous solvent) can be provided at any temperatures provided that the temperature does not induce any degradation of the peptides, change in peptide conformation, and/or any other undesirable effects on the peptides and/or resulting peptide nanostructures. In some embodiments, the formulation medium can have a temperature of about 0° C. to about 60° C., or about 2° C. to about 50° C., or about 4° C. to about room temperature.

Size and/shapes of self-assembled nanostructures formed can be controlled by the amount of peptide constructs added to the formulation medium (e.g., an aqueous solvent). In some embodiments, the concentration of the peptide constructs present in the formulation medium (e.g., an aqueous solvent) can range from about 0.1 mg/mL to about 1000 mg/mL, from about 0.5 mg/mL to about 750 mg/mL, from about 1 mg/mL to about 500 mg/mL, from about 2 mg/mL to about 250 mg/mL, from about 2 mg/mL to about 100 mg/mL, from about 2.5 mg/mL to about 50 mg/mL, from about 5 mg/mL to about 50 mg/mL. In some embodiments, the concentration of the peptide constructs present in the aqueous solvent can range from about 0.5 mg/mL to about 500 mg/mL. In some embodiments, the concentration of the peptide constructs present in the aqueous solvent can range from about 5 mg/mL to about 300 mg/mL.

In the solvent injection protocol described earlier, the peptide constructs are pre-dissolved in an organic solvent (e.g., but not limited to, dimethyl sulfoxide (DMSO), acetone, ethanol, dioxane, acetonitrile, methanol, THF, or any combinations thereof) at a higher concentration, prior to adding the isolated peptides to the formulation medium (e.g., an aqueous solvent). For example, the peptide constructs can be pre-dissolved in an organic solvent at a concentration in range of about 50 mg/mL to the maximum solubility of the peptide constructs in the selected organic solvent. By way of example only, the peptide constructs can be pre-dissolved in DMSO at a concentration of about 50 mg/mL to about 400 mg/mL, which is typically the maximum solubility of the peptide costructs in DMSO.

In some embodiments, the formed nanostructures can be further subjected to a post-treatment, e.g., to form a different nanostructure. Exemplary post-treatments can include, but are not limited to, flash-freezing followed by lyophilization and/or a series of ethanol/hexamethyldisilazine as shown in Example 5. Other post-treatments can include exposure to a solvent and/or coating a surface of the peptide nanostructures.

In some embodiments where it is desirable to form nanostructures comprising at least one additive distributed therein (e.g., but not limited to, active agent, a therapeutic agent, a preventative agent, a diagnostic agent, an imaging agent, a ligand, a labeling agent, and/or a substrate), the additive can be added into the mixture or the formulation medium containing self-assembling peptides prior to or during self-assembly process, for example, as shown in Example 8.

Alternatively or additionally, at least a subset of the self-assembling peptides can be conjugated to the additive of interest, prior to subjecting the self-assembling peptides to a formulation medium. Stated another way, the additive can be integrated directly or indirectly (e.g., via a linker or a conjugation or crosslinking agent described herein such as a binding molecule, a coupling molecule, a peptide-modifying molecule, and/or a cleavable linking groups or sequences) to the self-assembling peptide structure (e.g., the amino acid sequence of the self assembling peptides). In some embodiments where the additive is a peptide-based biologic, unitary peptide nanostructures, rather than nanoparticles that are formed and later covalently modified, can be generated. In some embodiments, the additive, e.g., a bioactive agent and/or a bioactive peptide, can be conjugated to the isolate peptide described herein via a linker agent that is cleavable to effectively make a nanoscale prodrug. In some embodiments where the linker or the conjugation or crosslinking agent is peptide-based, unitary peptide nanostructures, rather than nanoparticles that are formed and later covalently modified, can be generated.

In some embodiments where at least one cell is encapsulated in a nanostructure, one or more cells can be added to an aqueous solution containing self-assembling peptides with a suitable isotonicity and/or pH (e.g., to support cell viability and/or proliferation) prior to or during self-assembly process. In some embodiments, cell medium or nutrients (e.g., growth factors) can be included in the aqueous solution, e.g., to support cell viability and/or proliferation.

### Personal Care Compositions

In some embodiments, the isolated peptides and/or peptide nanostructures can be provided in different types of personal care compositions. In one embodiment, the personal care composition can be formulated to be a hair care composition selected from the group consisting of shampoo, conditioner, anti-dandruff treatments, styling aids, styling conditioner, hair repair or treatment serum, lotion, cream, pomade, and chemical treatments. In another embodiment, the styling aids are selected from the group consisting of spray, mousse, rinse, gel, foam and a combination thereof. In another embodiment, the chemical treatments are selected from the group consisting of permanent waves, relaxers, and permanent, semi-permanent, and temporary color treatments and combinations thereof.

In another embodiment, the personal care composition can be formulated to be a skin care composition selected from the group consisting of moisturizing body wash, body wash, antimicrobial cleanser, skin protectant treatment, body lotion, facial cream, moisturizing cream, facial cleansing emulsion, surfactant-based facial cleanser, facial exfoliating gel, facial toner, exfoliating cream, facial mask, after shave balm and sunscreen.

In another embodiment, the personal care composition can be formulated to be a cosmetic composition selected from the group consisting of eye gel, lipstick, lip gloss, lip balm, mascara, eyeliner, pressed powder formulation, foundation, fragrance and/or solid perfume. In a further embodiment, the cosmetic composition comprises a makeup composition. Makeup compositions include, but are not limited to color cosmetics, such as mascara, lipstick, lip liner, eye shadow, eye liner, rouge, face powder, make up foundation, and nail polish.

In yet another embodiment, the personal care composition can be formulated to be a nail care composition in a form selected from the group consisting of nail enamel, cuticle treatment, nail polish, nail treatment, and polish remover.

In yet another embodiment, the personal care composition can be formulated to be an oral care composition in a form selected from the group consisting of toothpaste, mouth rinse, breath freshener, whitening treatment, and inert carrier substrates.

The personal care composition can be in any form to suit the need of an application and/or preference of users. For example, the personal care composition can be in the form of an emulsified vehicle, such as a nutrient cream or lotion, a stabilized gel or dispersioning system, such as skin softener, a nutrient emulsion, a nutrient cream, a massage cream, a treatment serum, a liposomal delivery system, a topical facial pack or mask, a surfactant-based cleansing system such as a shampoo or body wash, an aerosolized or sprayed dispersion or emulsion, a hair or skin conditioner, styling aid, or a pigmented product such as makeup in liquid, cream, solid, anhydrous or pencil form.

In some embodiments of various kinds of the personal care composition described herein, the composition can further comprise an active ingredient or an active agent described herein. One skilled in the art will appreciate the various active ingredients or active agents for use in personal care compositions, any of which may be employed herein, see e.g., McCutcheon's Functional Materials, North American and International Editions, (2003), published by MC Publishing Co. For example, the personal care compositions herein can comprise a skin care active ingredient at a level from about 0.0001% to about 20%, by weight of the composition. In another embodiment, the personal care composition comprises a skin care active ingredient from about 0.001% to about 5%, by weight of the composition. In yet another embodiment, the personal care composition comprises a skin care active ingredient from about 0.01% to about 2%, by weight of the composition.

In some embodiments, the isolated peptides and/or peptide nanostructures can be used to stabilize and/or provide a controlled release or sustained release of at least one skin care active ingredient. Skin care active ingredients include, but are not limited to, antioxidants, such as tocopheryl and ascorbyl derivatives; retinoids or retinols; essential oils; bioflavinoids, terpenoids, synthetics of biolflavinoids and terpenoids and the like; vitamins and vitamin derivatives; hydroxyl- and polyhydroxy acids and their derivatives, such as AHAs and BHAs and their reaction products; peptides and polypeptides and their derivatives, such as glycopeptides and lipophilized peptides, heat shock proteins and cytokines; enzymes and enzymes inhibitors and their derivatives, such as proteases, MMP inhibitors, catalases, CoEnzyme Q10, glucose oxidase and superoxide dismutase (SOD); amino acids and their derivatives; bacterial, fungal and yeast fermentation products and their derivatives, including mushrooms, algae and seaweed and their derivatives; phytosterols and plant and plant part extracts; phospholipids and their derivatives; anti-dandruff agents, such as zinc pyrithione, and chemical or organic sunscreen agents such as ethylhexyl methoxycinnamate, avobenzone, phenyl benzimidazole sulfonic acid, and/or zinc oxide. Delivery systems comprising the active ingredients are also provided herein.

In addition to the active ingredients noted above, the personal care composition can further comprise a physiologically acceptable carrier or excipient. Specifically, the personal care compositions herein can comprise a safe and effective amount of a dermatologically acceptable carrier, suitable for topical application to the skin or hair within which the essential materials and optional other materials are incorporated to enable the essential materials and optional components to be delivered to the skin or hair at an appropriate concentration. The carrier can thus act as a diluent, dispersant, solvent or the like for the essential components which ensures that they can be applied to and distributed evenly over the selected target at an appropriate concentration.

An effective amount of one or more compounds described herein can also be included in personal care compositions to be applied to keratinous materials such as nails and hair, including but not limited to those useful as hair spray compositions, hair styling compositions, hair shampooing and/or conditioning compositions, compositions applied for the purpose of hair growth regulation and compositions applied to the hair and scalp for the purpose of treating seborrhea, dermatitis and/or dandruff.

An effective amount of one or more compounds described herein may be included in personal care compositions suitable for topical application to the skin, teeth, nails or hair. These compositions can be in the form of creams, lotions, gels, suspensions dispersions, microemulsions, nanodispersions, microspheres, hydrogels, emulsions (e.g., oil-in-water and water-in-oil, as well as multiple emulsions) and multilaminar gels and the like (see, for example, The Chemistry and Manufacture of Cosmetics, Schlossman et al., 1998), and can be formulated as aqueous or silicone compositions or can be formulated as emulsions of one or more oil phases in an aqueous continuous phase (or an aqueous phase in an oil phase).

A variety of optional ingredients such as neutralizing agents, fragrance, perfumes and perfume solubilizing agents, coloring agents, surfactants, emulsifiers, and/or thickening agents can also be added to the personal care compositions herein. Any additional ingredients should enhance the product, for example, the skin softness/smoothness benefits of the product. In addition, any such ingredients should not negatively impact the aesthetic properties of the product.

Suitably, the pH of the personal care compositions herein is in the range from about 3.5 to about 10, specifically from about 4 to about 8, and more specifically from about 5 to about 7, wherein the pH of the final composition is adjusted by addition of acidic, basic or buffer salts as necessary, depending upon the composition of the forms and the pH-requirements of the compounds.

One skilled in the art will appreciate the various techniques for preparing the personal care compositions of the present invention, any of which may be employed herein.

### Pharmaceutical Compositions

For administration to a subject in vivo, peptide nanostructures comprising a therapeutic agent and an active agent described herein can be provided in pharmaceutically acceptable compositions. These pharmaceutically acceptable compositions comprise a nanostructure or an active agent—self-assembling peptide complex formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, mannose, fructose, dextrose, trehalose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

As used herein, the term “administer” refers to the placement of a composition into a subject by a method or route which results in at least partial localization of the composition at a desired site such that desired effect is produced. Routes of administration include both local and systemic administration. Generally, local administration results in more of the therapeutic agent being delivered to a specific location as compared to the entire body of the subject, whereas, systemic administration results in delivery of the therapeutic agent to essentially the entire body of the subject.

Administration to a subject can be by any appropriate route known in the art including, but not limited to, parenteral routes, pulmonary routes, enteral routes, topical routes, or any combinations thereof. Examples of administration routes can include, but are not limited to, intravenous, intramuscular, subcutaneous, transdermal, airway (aerosol), pulmonary, nasal, oral, ocular, buccal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments of the aspects described herein, administration is by intravenous infusion or injection.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders or diseases. In addition, the methods and compositions described herein can be used to treat domesticated animals and/or pets.

Embodiments of various aspects described herein can be defined in any of the following numbered paragraphs:
1. An isolated peptide consisting essentially of:
   an amino acid sequence of (Y₁-X₁-X₂-X₃-X₄-X₃-Y₂)ₙ conjugated to at least one entity, wherein
      X₁ is valine (Val) or a conservative substitution thereof;
      X₂ is proline (Pro) or a conservative substitution thereof;
      X₃ is glycine (Gly) or a conservative substitution thereof;
   X₄ in each nth unit is independently an amino acid residue, wherein when n is 4, at least one X₄ is not valine;
   Y₁ and Y₂ are each independently a linker, wherein the linker is selected from a bond, one amino acid residue or a group of amino acid residues, wherein the combined amino acid sequences of Y₁ and Y₂ does not comprise a sequence of (VPGX₄G);
   n is an integer from 1 to 50; and
   the entity is selected from a group consisting of —H, —OH, a chemical functional group, a ligand, an active agent, a therapeutic agent, a binding molecule, a coupling molecule, a labeling agent, a peptide-modifying molecule, and a substrate, wherein when the amino acid sequence is a repeated sequence of (VPGVG), the substrate is not a biodegradable non-amino acid moiety.
2. The isolated peptide of paragraph 1, wherein when Y₁ and Y₂ are each a bond, the isolated peptide consists essentially of the amino acid sequence of (X₁-X₂-X₃-X₄-X₃)ₙ conjugated to said at least one entity.
3. The isolated peptide of paragraph 2, wherein when said at least one entity is —H or —OH, the isolated peptide consists essentially of H—(X₁-X₂-X₃-X₄-X₃)ₙ—OH.
4. The isolated peptide of paragraph 1 or 2, wherein the chemical functional group is selected from the group consisting of alkyne, halogens, alcohol, ketone, aldehyde, acyl halide, carbonate, carboxylate, carboxylic acid, ester, hydroperoxide, peroxide, ether, hemiacetal, hemiketal, acetal, ketal, acetal, orthoester, amide, amines, imine, imide, azide, azo compound, cyanates, nitrate, nitrile, nitrite, nitro compound, nitroso compound, pyridine, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, borinic ester, and any combinations thereof.
5. The isolated peptide of paragraph 1 or 2, wherein the peptide-modifying molecule includes a polypeptide sequence comprising amino acids Pro, Ala, and Ser; a hydroxyethyl starch (HES) derivative; and a combination thereof.
6. The isolated peptide of any of paragraphs 1-5, wherein the amino acid sequence is (Y₁-Val-Pro-Gly-X₄-Gly-Y₂)ₙ, wherein each amino acid residue is independently a D-amino acid or a L-amino acid.
7. The isolated peptide of any of paragraphs 1-6, wherein at least one of the amino acid residues in the amino acid sequence is a non-proteinogenic or non-standard amino acid.
8. The isolated peptide of any of paragraphs 1-7, wherein n is an integer from 1 to 25.
9. The isolated peptide of any of paragraphs 1-8, wherein n is an integer from 1 to 10.
10. The isolated peptide of any of paragraphs 1-9, wherein n is an integer from 1 to 2.
11. The isolated peptide of any of paragraphs 1-10, wherein at least one X₄ in the amino acid sequence is different from another X₄ in the amino acid sequence.
12. The isolated peptide of any of paragraphs 1-11, wherein at least one X₄ is a hydrophobic amino acid.
13. The isolated peptide of any of paragraphs 1-12, wherein at least two X₄'s are hydrophobic amino acids.
14. The isolated peptide of any of paragraphs 1-13, wherein the X₄ is selected from the group consisting of phenylalanine (Phe), isoleucine (Ile), leucine (Leu), tyrosine (Tyr), tryptophan (Trp), valine (Val), lysine (Lys), histidine (His), methionine (Met), a non-standard amino acid, a side-chain modified amino acid, and a derivative thereof.
15. The isolated peptide of any of paragraphs 1-14, wherein the amino acid sequence is selected from the group consisting of

| | |
|---|---|
| a. | Val-Pro-Gly-Phe-Gly-Val-Pro-Gly-Phe-Gly; |
| b. | Val-Pro-Gly-Ile-Gly-Val-Pro-Gly-Leu-Gly; |
| c. | Val-Pro-Gly-Tyr-Gly-Val-Pro-Gly-Phe-Gly; |
| d. | Val-Pro-Gly-Phe-Gly-Val-Pro-Gly-Tyr-Gly; |
| e. | Val-Pro-Gly-Trp-Gly-Val-Pro-Gly-Phe-Gly; |
| f. | Val-Pro-Gly-Phe-Gly-Val-Pro-Gly-Trp-Gly; |
| g. | Val-Pro-Gly-Tyr-Gly-Val-Pro-Gly-Tyr-Gly; |
| h. | Val-Pro-Gly-Trp-Gly-Val-Pro-Gly-Trp-Gly; |
| i. | Val-Pro-Gly-Phe-Gly; |
| j. | Val-Pro-Gly-Tyr-Gly; |
| k. | Val-Pro-Gly-Trp-Gly; |
| l. | Val-Pro-Ala-Tyr-Gly; |
| m. | Ala-Pro-Gly-Tyr-Gly; |
| n. | Ile-Pro-Gly-Tyr-Gly; |
| and | |
| o. | Leu-Pro-Gly-Tyr-Gly. |

16. The isolated peptide of any of paragraphs 1-15, wherein the ligand is selected from a group consisting of a cell surface receptor ligand, a ligand, an antibody or a portion thereof, an antibody-like molecule, an enzyme, an antigen, a small molecule, a protein, a peptide, a peptidomimetic, a nucleic acid molecule, a carbohydrate, an aptamer, a cytokine, a lectin, a lipid, a plasma albumin, and any combinations thereof.
17. The isolated peptide of any of paragraphs 1-16, wherein the binding molecule includes biotin, avidin, streptavidin, immunoglobulin, protein A, protein G, hormone, receptor, receptor antagonist, receptor agonist, enzyme, enzyme cofactor, enzyme inhibitor, a charged molecule, carbohydrate, lectin, steroid, or any combinations thereof.
18. The isolated peptide of any of paragraphs 1-17, wherein the substrate includes a gold particle, a silver particle, a magnetic particle, a quantum dot, a fullerene, a carbon tube, a nanowire, a nanofibril, a grapheme, and any combinations thereof.
19. The isolated peptide of any of paragraphs 1-18, wherein the substrate includes collagen, albumin, silk, hyaluronic acid, and any combination thereof.
20. The isolated peptide of any of paragraphs 1-19, wherein the substrate includes a polymer.
21. The isolated peptide of any of paragraphs 1-20, wherein when the N-terminus or C-terminus of the amino acid sequence is not conjugated to said at least one entity, Y₁ or Y₂ located at the N-terminus or C-terminus is absent.
22. The isolated peptide of any of paragraphs 1-21, wherein the substrate is not a biodegradable non-amino acid moiety for any integer of n.
23. A self-assembled peptide nanostructure comprising a plurality of isolated peptides of any of paragraphs 1-22.
24. The self-assembled peptide nanostructure of paragraph 23, further comprising a biopolymer.
25. The self-assembled peptide nanostructure of paragraph 24, wherein the biopolymer is conjugated to at least one of the isolated peptides and/or the self-assembled peptide nanostructure.
26. The self-assembled peptide nanostructure of any of paragraphs 23-25, further comprising an active agent, a ligand, a labeling agent, or any combinations thereof.
27. The self-assembled peptide nanostructure of paragraph 26, wherein the active agent, the ligand, the labeling agent, or any combinations thereof is conjugated to at least one of the isolated peptides and/or the self-assembled peptide nanostructure.
28. The self-assembled peptide nanostructure of any of paragraphs 23-27, wherein the nanostructure is in a form of a particle, a fiber, a rod, a ring, an aggregate, a vesicle, a prism, a gel, or any combinations thereof.
29. The self-assembled peptide nanostructure of any of paragraphs 23-28, wherein the nanostructure is porous.
30. The self-assembled peptide nanostructure of any of paragraphs 23-29, wherein the nanostructure has a solid structure.
31. The self-assembled peptide nanostructure of any of paragraphs 23-29, wherein the nanostructure has a hollow core structure surrounded by a shell.
32. The self-assembled peptide nanostructure of any of paragraphs 23-31, wherein the nanostructure comprises a laminar structure.
33. The self-assembled peptide nanostructure of any of paragraphs 23-32, wherein the nanostructure has a size of about 10 nm to about 500 μm.
34. The self-assembled peptide nanostructure of any of paragraphs 23-33, wherein the isolated peptides are selected such that the self-assembled peptide nanostructure maintain its shape and/or size for a period of at least about 6 hours, at least about 12 hours, at least about 1 day, or at least about 5 days.
35. An article comprising an isolated peptide of any of paragraphs 1-22, a self-assembled peptide nanostructure of any of paragraphs 23-34, or any combination thereof.
36. The article of paragraph 35, wherein the article is selected from the group consisting of a tissue engineered scaffold, a medication, a therapeutic agent, a preventative agent, a diagnostic agent, an imaging agent, a coating of a medical device, a delivery device or vehicle, and any combinations thereof.
37. A composition comprising an isolated peptide of any of paragraphs 1-22, a self-assembled peptide nanostructure of any of paragraphs 23-34, or any combination thereof.
38. The composition of paragraph 37, wherein the isolated peptide is present in a first amount sufficient to alter at least one property of the composition.
39. The composition of paragraph 37 or 38, wherein the self-assembled peptide nanostructure is present in a second amount sufficient to alter at least one property of the composition.
40. The composition of any of paragraphs 38-39, wherein said at least one property of the composition includes consistency, stability, absorption, nutrient value, therapeutic potential, esthetics, flavor, olfactory property, material property, bioavailability, or any combinations thereof.
41. The composition of any of paragraphs 37-40, wherein the composition is a food composition.
42. The composition of any of paragraphs 37-40, wherein the composition is a pharmaceutical composition.
43. The composition of paragraph 42, wherein the pharmaceutical composition is formulated for oral administration.
44. The composition of paragraph 42, wherein the pharmaceutical composition is formulated for parenteral administration.
45. The composition of any of paragraphs 37-40, wherein the composition is a personal care composition.
46. The composition of paragraph 45, wherein the personal care composition is a hair care composition or a skin care composition.
47. The composition of paragraph 46, wherein the hair care composition or the skin care composition is a cream, oil, lotion, powder, serum, gel, shampoo, conditioner, ointment, foam, spray, aerosol, mousse, or any combinations thereof.
48. The composition of any of paragraphs 37-40, wherein the composition is a cosmetic composition.
49. The composition of paragraph 48, wherein the cosmetic composition is powder, lotion, cream, lipstick, nail varnish, hair dye, balm, spray, mascara, fragrance, solid perfume, or any combinations thereof.
50. A food additive comprising an isolated peptide of any of paragraphs 1-22, a self-assembled peptide nanostructure of any of paragraphs 23-34, or any combination thereof.
51. The food additive of paragraph 50, wherein the isolated peptide is configured to be capable of altering at least one property of a food composition upon addition of the isolated peptide to the food composition.
52. The food additive of paragraph 50 or 51, wherein the peptide nanostructure is configured to be capable of altering at least one property of a food composition upon addition of the peptide nanostructure to the food composition.
53. The food additive of any of paragraphs 50-52, wherein said at least one property of the food composition includes consistency, stability, absorption, nutrient value, esthetics, flavor, olfactory property, material property, or any combinations thereof.
54. A kit comprising at least one container containing an isolated peptide of any of paragraphs 1-22, or a self-assembled peptide nanostructure of any of paragraphs 23-34, and at least one reagent.
55. The kit of paragraphs 54, further comprising an active agent.
56. A method of modulating at least one behavior of a biological cell comprising contacting the cell with a composition comprising at least one isolated peptide of any of paragraphs 1-22, at least one peptide nanostructure of any of paragraphs 23-34, or any combination thereof.
57. The method of paragraph 56, wherein said at least one behavior of the cell includes growth, viability, migration, differentiation, secretion, protein synthesis, apoptosis, fate switching, contractibility, or any combinations thereof.
58. The method of paragraph 56 or 57, wherein the biological cell is present in vitro.
59. The method of paragraph 56 or 57, wherein the biological cell is present in a subject.
60. The method of paragraph 59, wherein said contacting the cell with the composition comprises administering the subject with the composition.
61. The method of paragraph 60, wherein the administration includes oral administration and/or parenteral administration.
62. A method of modulating release of an active agent from a composition or an article comprising:
   providing a composition or an article comprising an active agent and peptide nanostructures of any of paragraphs 23-34, wherein the active agent is distributed in at least one of the peptide nanostructures, and wherein at least a portion of the peptide nanostructures are capable of responding to at least one stimulus; and
   exposing the peptide nanostructures to said at least one stimulus, wherein the response of the peptide nanostructures to said at least one stimulus modulates the release of the active agent from the peptide nanostructures.
63. The method of paragraph 62, wherein the active agent is encapsulated within the peptide nanostructures.
64. The method of paragraph 62 or 63, wherein the active agent is conjugated to the isolated peptide of any of paragraphs 1-22 forming the peptide nanostructures.
65. The method of any of paragraphs 62-64, wherein the response of the peptide nanostructures to said at least one stimulus includes a change in size, pore size or porosity of the peptide nanostructures, a change in interaction between the peptide nanostructures and at least one component of the matrix, or any combinations thereof.
66. The method of any of paragraphs 62-65, wherein said at least one stimulus is selected from the group consisting of a change in light intensity and/or wavelength, a change in pH, a change in temperature, a change in humidity, and any combinations thereof.
67. The method of any of paragraphs 62-66, wherein the peptide nanostructure is in a form of a particle, a rod, a prism, a disc, a fiber, a vesicle, a ring, or any combinations thereof.
68. The method of any of paragraphs 62-67, wherein the composition or article is in a form of a gel, a scaffold, a film, a patch, a particle, a cream, an ointment, a solution, a capsule, a pill, a tablet, powder, a paste, or any combinations thereof.
69. A method of modulating at least one material property and/or structure of a matrix comprising:
   providing a matrix comprising a plurality of the isolated peptides of any of paragraphs 1-22 and/or the peptide nanostructures of any of paragraphs 23-34, wherein at least a portion of the isolated peptides and/or the peptide nanostructures are capable of responding to at least one stimulus; and
   exposing the isolated peptides and/or the peptide nanostructures to said at least one stimulus, wherein the response of the isolated peptides and/or the peptide nanostructure to said at least one stimulus modulates said at least one material property and/or structure of the matrix.
70. The method of paragraph 69, wherein said at least one material property of the matrix is selected from the group consisting of viscosity, porosity, mechanical stiffness, ductility, viscoelasticity, organization, degradability, solubility, density, flexibility, permeability, hydrophobicity, optical properties, thermal properties, and any combinations thereof.
71. The method of paragraph 69, wherein said at least one material property of the matrix includes mechanical stiffness and/or viscoelasticity.
72. The method of any of paragraphs 69-71, wherein the response of the isolated peptides within the matrix includes a conformational change, a change in interaction between the isolated peptides within the matrix, a change in interaction between the isolated peptides and at least one component of the matrix, size and/or shape of the peptide nanostructures formed from the isolated peptides, or any combinations thereof.
73. The method of any of paragraphs 69-72, wherein the response of the peptide nanostructures within the matrix includes a change in size, shape, pore size, or porosity of the peptide nanostructures within the matrix, a change in interaction between the peptide nanostructures and at least one component of the matrix, or any combinations thereof.
74. The method of any of paragraphs 69-73, wherein said at least one stimulus is selected from the group consisting of a change in light intensity and/or wavelength, a change in pH, a change in temperature, a change in humidity, and any combinations thereof.
75. The method of any of paragraphs 69-74, wherein the peptide nanostructures are in a form of a particle, a rod, a prism, a disc, a fiber, a vesicle, a ring, or any combinations thereof.
76. The method of any of paragraphs 69-75, further comprising introducing the isolated peptides and/or the peptide nanostructures into the matrix.
77. The method of paragraph 76, wherein the isolated peptides and/or the peptide nanostructures are conjugated to the matrix.
78. The method of paragraph 76, wherein the isolated peptides and/or the peptide nanostructures are entrapped in the matrix.
79. The method of any of paragraphs 69-78, wherein the matrix is a scaffold, a gel, a cell or a tissue.
80. A method of inducing gel formation of a protein or polymer comprising:
   providing a solution or suspension of a protein or polymer, wherein the protein or polymer molecules are conjugated to at least one isolated peptide of any of paragraphs 1-22, and wherein said at least one isolated peptide is capable of responding to at least one stimulus; and
   exposing the isolated peptide within the solution or suspension to said at least one stimulus, wherein the response of the isolated peptides conjugated to the protein or polymer molecules induces aggregation of the protein or polymer molecules to form a gel.
81. The method of paragraph 80, wherein said at least one stimulus is selected from the group consisting of a change in light intensity and/or wavelength, a change in pH, a change in temperature, a change in humidity, and any combinations thereof.
82. A method of altering at least one property of food or a food composition comprising:
   providing food or a food composition comprising an effective amount of the isolated peptides of any of paragraphs 1-22 and/or the peptide nanostructures of any of paragraphs 23-34, wherein the effective amount is sufficient to alter at least one property of the food or the food composition.
83. The method of paragraph 82, wherein at least a portion of the isolated peptides and/or the peptide nanostructures are capable of responding to at least one stimulus.
84. The method of paragraph 82 or 83, further comprising exposing the isolated peptides and/or the peptide nanostructures to said at least one stimulus, wherein the response of the isolated peptides and/or the peptide nanostructures to said at least one stimulus alters said at least one property of the food or the food composition.
85. The method of paragraph 84, wherein the response of the isolated peptides includes a conformational change, a change in interaction between the isolated peptides within the food or food composition, a change in interaction between the isolated peptides and at least one component of the food or food composition, size and/or shape of the peptide nanostructures formed from the isolated peptides, or a combinations thereof.
86. The method of any of paragraphs 82-85, wherein the response of the peptide nanostructures includes a change in size, shape, pore size, and/or porosity of the nanostructures within the food or food composition, a change in interaction between the peptide nanostructures and at least one component of the food or food composition.
87. The method of any of paragraphs 82-86, wherein said at least one stimulus is selected from the group consisting of a change in light intensity and/or wavelength, a change in pH, a change in temperature, a change in humidity, and any combinations thereof.
88. The method of any of paragraphs 82-87, wherein the peptide nanostructures are in a form of a particle, a rod, a prism, a disc, a fiber, a vesicle, a ring, or any combinations thereof.
89. The method of any of paragraphs 82-88, further comprising contacting the food or the food composition with the effective amount of the isolated peptides and/or the peptide nanostructures.
90. The method of any of paragraphs 82-89, wherein said at least one property of the food or food composition includes consistency, stability, absorption, nutrient value, esthetics, flavor, olfactory property, material property, or any combinations thereof.
91. A method of forming peptide nanostructures comprising
   contacting the isolated peptides of any of paragraphs 1-22 with a pre-determined formulation medium, whereby the isolated peptides self-organize to form peptide nanostructures in the pre-determined formulation medium, wherein at least one property of the peptide nanostructures is determined by a parameter selected from the group consisting of composition and/or property of the pre-determined formulation medium, the amino acid sequence of the isolated peptides, concentration of the isolated peptides, and any combinations thereof.
92. The method of paragraph 91, wherein said at least one property of the peptide nanostructures includes average size, size distribution, shape, porosity, pore size, stability, mechanical property, and any combinations thereof.
93. The method of paragraph 91 or 92, wherein the pre-determined formulation medium has a temperature in a range of about 0° C. to about 60° C. or 4° C. to about 50° C.
94. The method of any of paragraphs 91-93, wherein the pre-determined formulation medium has a pH value in a range of about pH ˜1 to pH ˜14.
95. The method of any of paragraphs 91-94, wherein the pre-determined formulation medium is an aqueous medium.
96. The method of paragraph 95, wherein the pre-determined formulation medium further comprises salt.
97. The method of any of paragraphs 91-96, wherein the pre-determined formulation medium further comprises an additive.
98. The method of any of paragraphs 91-97, wherein at least a subset of the isolated peptides are conjugated to an additive.
99. The method of paragraph 97 or 98, wherein the additive includes an active agent, a ligand, a therapeutic agent, a labeling agent, a substrate, or any combinations thereof.
100. The method of paragraph 98 or 99, wherein said at least a first subset of the isolated peptides further comprise a linker sequence between the individual isolated peptides and the additive.
101. The method of paragraph 100, wherein the linker sequence comprises a cleavable sequence.
102. The method of any of paragraphs 98-101, further comprising conjugating the additive to said at least the first subset of the isolated peptides prior to said contacting the isolated peptides with the pre-determined formulation medium.
103. The method of paragraphs 98-102, wherein the additive forms part of the amino acid sequence of said at least the first subset of the isolated peptides.
104. The method of paragraph 102 or 103, wherein the additive is a bioactive peptide.
105. The method of any of paragraphs 91-104, wherein said contacting the isolated peptides with the pre-determined formulation medium comprises dissolving the isolated peptides in the pre-determined formulation medium.
106. The method of paragraph 105, wherein the isolated peptides are dissolved in the pre-determined formulation medium at a concentration in a range of about 0.5 mg/mL to about 500 mg/mL or about 5 mg/mL to about 300 mg/mL.
107. The method of any of paragraphs 91-104, wherein said contacting the isolated peptides with the pre-determined formulation medium comprises adding the isolated peptides in aliquots of a fixed volume to the pre-determined formulation medium.
108. The method of paragraph 107, wherein the isolated peptides are pre-dissolved in an organic solvent at a concentration in a range of about 50 mg/mL to the maximum solubility of the isolated peptides in the organic solvent, prior to said adding the isolated peptides to the pre-determined formulation medium.
109. The method of paragraph 107 or 108, wherein the ratio of the fixed volume to the volume of the pre-determined formulation medium is in a range from about 1:20 to about 1:1.
110. The method of any of paragraphs 91-109, further comprising subjecting at least a second subset of the formed peptide nanostructures to a post-treatment.
111. The method of paragraph 110, wherein the post-treatment includes flash-freezing, lyophilization, exposure to a solvent, surface coating, or any combinations thereof.
112. The method of any of paragraphs 91-111, wherein the peptide nanostructures have a size in a range of about 10 nm to about 500 μm.
113. The method of any of paragraphs 91-112, wherein the peptide nanostructures are polydispersed.
114. The method of any of paragraphs 91-112, wherein the peptide nanostructures are monodispersed.
115. The method of any of paragraphs 91-114, wherein the peptide nanostructures are in a form of a particle, a fiber, a rod, a ring, a prism, a vesicle, an aggregate, or any combinations thereof.
116. The method of any of paragraphs 91-115, wherein the peptide nanostructures comprise the isolated peptides having the same amino acid sequence.
117. The method of any of paragraphs 91-116, wherein the peptide nanostructures comprise the isolated peptides having different amino acid sequences.
118. An isolated peptide consisting essentially of:
   an amino acid sequence of (X₁-X₂-X₃-X₄-X₃)ₙ, wherein
      X₁ is valine (Val) or a conservative substitution thereof;
      X₂ is proline (Pro) or a conservative substitution thereof;
      X₃ is glycine (Gly) or a conservative substitution thereof;
   X₄ in each nth unit is independently an amino acid residue, wherein when n is 4, at least one X₄ is not valine; and
   n is an integer from 1 to 50.
119. An isolated peptide consisting essentially of:
   an amino acid sequence of (Y₁-X₁-X₂-X₃-X₄-X₃-Y₂)ₙ, wherein
      X₁ is valine (Val) or a conservative substitution thereof;
      X₂ is proline (Pro) or a conservative substitution thereof;
      X₃ is glycine (Gly) or a conservative substitution thereof;
   X₄ in each nth unit is independently an amino acid residue, wherein when n is 4, at least one X₄ is not valine;
   Y₁ and Y₂ are each independently a linker, wherein the linker is selected from a bond, one amino acid residue or a group of amino acid residues, wherein the combined amino acid sequences of Y₁ and Y₂ does not comprise a sequence of (VPGX₄G); and
   n is an integer from 1 to 50.
120. A conjugate comprising an isolated peptide of claim 118 or 119 conjugated to at least one agent.
121. The conjugate of claim 120, wherein said at least one agent is selected from the group consisting of a chemical functional group, a ligand, a therapeutic agent, a binding molecule, a coupling molecule, a labeling agent, a peptide-modifying molecule, and any combinations thereof.
122. The conjugate of claim 120 or 121, wherein said at least one agent includes a substrate, wherein when the amino acid sequence is a repeated sequence of (VPGVG), the substrate is not a biodegradable non-amino acid moiety.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not. Additionally, the term “comprising” or “comprises” includes “consisting essentially of” and “consisting of.”

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages can mean±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The term “statistically significant” or “significantly” refers to statistical significance and generally means a two standard deviation (2SD) above or below a reference level. The term refers to statistical evidence that there is a difference. It is defined as the probability of making a decision to reject the null hypothesis when the null hypothesis is actually true. The decision is often made using the p-value.

As used interchangeably herein, the terms “non-proteinogenic amino acid” and “non-standard amino acid” refers to an organic compound that is not among those encoded by the standard genetic code, or incorporated into proteins during translation. The non-proteinogenic amino acid or non-standard amino acid can be prepared synthetically or derived from a natural source. Non-proteinogenic amino acids, thus, include amino acids or analogs of amino acids other than the 22 proteinogenic or standard amino acids used for protein biosynthesis and include, but are not limited to, the D-isomers of proteinogenic amino acids. As used herein, the term “proteinogenic amino acids” refers to amino acids used for protein biosynthesis as well as other amino acids that can be incorporated into proteins during translation (including pyrrolysine and selenocysteine). Examples of proteinogenic amino acids include the twenty-two standard amino acids, e.g., glycine, alanine, valine, leucine, isoleucine, aspartic acid, glutamic acid, threonine, glutamine, asparagine, arginine, lysine, proline, phenylalanine, tyrosine, tryptophan, cysteine, methionine, and histidine, and selenocysteine and pyrrolysine.

In some embodiments, the non-proteinogenic amino acid can be classified as (i) homo analogues of proteinogenic amino acids; (ii) β-homo analogues of proteinogenic amino acid residues and (iii) other non-proteinogenic amino acid residues.

For example, homo analogues of proteinogenic amino acids include the ones where the side chain has been extended by a methylene group, e.g., homoalanine (Hal), homoarginine (Har), homocysteine (Hey), homoglutamine (Hgl), homohistidine (Hhi), homoisoleucine (Hil), homoleucine (Hie), homolysine (Hly), homomethionine (Hme), homophenylalanine (Hph), homoproline (Hpr), homoserine (Hse), homothreonine (Hth), homotryptophane (Htr), homotyrosine (Hty) and homovaline (Hva).

Non-limiting examples of β-homo analogues of proteinogenic amino acids include the ones where a methylene group has been inserted between the α-carbon and the carboxyl group yielding β-amino acids, e.g., β-homoalanine (βHal), β-homoarginine (βHar), β-homoasparagine (βHas), β-homocysteine (βHcy), β-homoglutamine (βHgl), β-homohistidine (βHhi), β-homoisoleucine (βHil), β-homoleucine (βHle), β-homolysine (βHly), β-homomethionine (βHme), β-homophenylalanine (βHph), β-homoproline (βHpr), β-homoserine (βHse), β-homothreonine (βHth), β-homotryptophane (βHtr), β-homotyrosine (βHty) and β-homovaline (βHva).

Other examples of non-proteinogenic amino acids include, but are not limited to, ring-substituted phenylalanine or tyrosine, and tryptophan derivatives (e.g., but not limited to, fluoro/chloro/bromo/iodo/cyano/borono-phenylalanine, DL-o-tyrosine, DL-m-Tyrosine purum, fluoro-tryptophan, hydroxy-tryptophan, methoxy-tryptophan), citrulline, homocitrulline, α-aminoadipic acid (Aad), β-aminoadipic acid (βAad), α-aminobutyric acid (Abu), α-aminoisobutyric acid (Aib), β-alanine (βAla), 4-aminobutyric acid (4-Abu), 5-aminovaleric acid (5-Ava), 6-aminohexanoic acid (6-Ahx), 8-aminooctanoic acid (8-Aoc), 9-aminononanoic acid (9-Anc), 10-aminodecanoic acid (10-Adc), 12-aminododecanoic acid (12-Ado), α-aminosuberic acid (Asu), azetidine-2-carboxylic acid (Aze), β-cyclohexylalanine (Cha), aitrulline (Cit), dehydroalanine (Dha), γ-carboxyglutamic acid (Gla), α-cyclohexylglycine (Chg), propargylglycine (Pra), pyroglutamic acid (Gip), α-tertbutylglycine (Tie), 4-benzoylphenylalanine (Bpa), 8-hydroxylysine (Hyl), 4-hydroxyproline (Hyp), allo-isoleucine (alle), lanthionine (Lan), (1-naphthyl) alanine (1-NaI), (2-naphthyl)alanine (2-NaI), norleucine (Nle), norvaline (Nva), ornithine (Orn), phenylglycin (Phg), pipecolic acid (Pip), sarcosine (Sar), selenocysteine (Sec), statine (Sta), β-thienylalanine (Thi), 1,2,3,4-tetrahydroisochinoline-3-carboxylic acid (Tic), allo-threonine (aThr), thiazolidine-4-carboxylic acid (Thz), γ-aminobutyric acid (GABA), isocysteine (iso-Cys), diaminopropionic acid (Dpr), 2,4diaminobutyric acid (Dab), 3,4-diaminobutyric acid (γβDab), biphenylalanine (Bip), phenylalanine substituted in para-position with —C₁-C₆ alkyl, -halide, —NH₂, —CO₂H or Phe(4-R) (wherein R=—C₁-C₆ alkyl, -halide, —NH₂, or —CO₂H); peptide nucleic acids (PNA, cf, P. E. Nielsen, Acc. Chem. Res., 32, 624-30); or their N-alkylated analogues, such as their N-methylated analogues.

As used herein, the term “non-proteinogenic amino acid” can also encompass derivatives of proteinogenic amino acids. For example, the side chain, C-terminus and/or the N-terminus of a proteinogenic amino acid residue can be derivatized thereby rendering the proteinogenic amino acid residue “non-proteinogenic.”

The term “nanosphere” means a particle having an aspect ratio of at most 3:1. The term “aspect ratio” means the ratio of the longest axis of an object to the shortest axis of the object, where the axes are not necessarily perpendicular.

The term “nanorod” means a particle having a longest dimension of at most 200 nm, and having an aspect ratio of from 3:1 to 20:1.

The term “nanoprism” means a particle having at least two non-parallel faces connected by a common edge.

As used herein, the “diameter” of a particle means the average of the diameters of the nanoparticle.

The “average” dimension of a plurality of particles means the average of that dimension for the plurality. For example, the “average diameter” of a plurality of nanospheres means the average of the diameters of the nanospheres, where a diameter of a single nanosphere is the average of the diameters of that nanosphere.

As used herein, the term “pharmaceutically-acceptable salts” refers to the conventional nontoxic salts or quaternary ammonium salts of a compound, e.g., from non-toxic organic or inorganic acids. These salts can be prepared in situ in the administration vehicle or the dosage form manufacturing process, or by separately reacting a purified compound in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In some embodiments of the aspects described herein, representative salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

As used herein, a “ratio” can be a mol ratio or weight ratio.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Design of Exemplary Self-Assembling Peptides (e.g., 5-10 Amino Acids)

There is still a need for new synthetic materials, or new ways to manipulate existing materials, to fulfill unmet needs, for example, in drug delivery and tissue engineering [1-10]. For example, current unmet needs in the drug development arena include, but are not limited to, reducing drug toxicity, improving pharmacokinetics (PK), enhancing drug efficacy, targeting agents selectively to disease sites, delivering drugs to intracellular targets, and any combinations thereof [1,11]. Some existing biodegradable scaffolds lack cell-specific bioactivities, such as cell adhesion and migration[12].

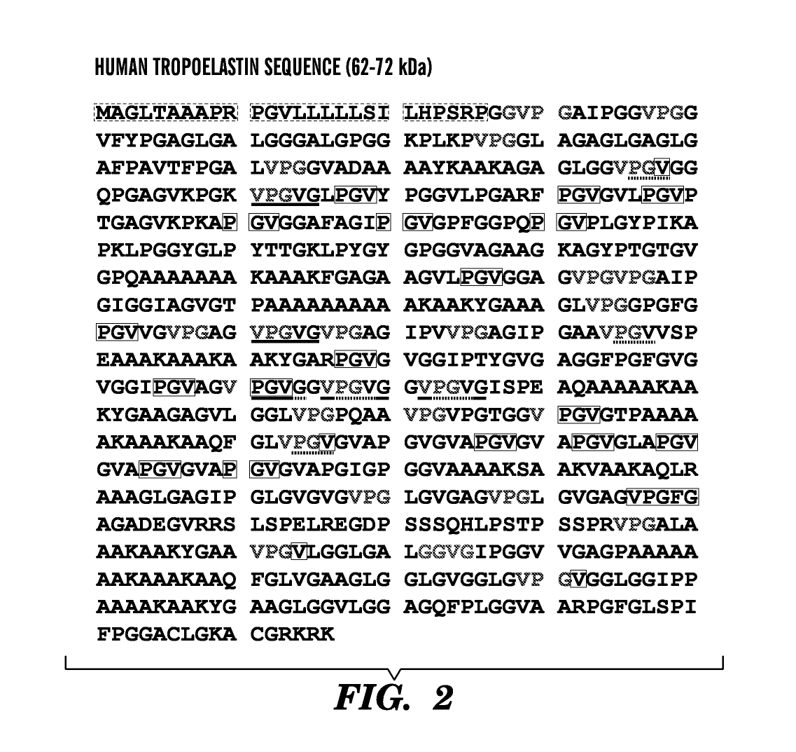

Tropoelastin is a ˜70 kDa precursor soluble protein that spontaneously self-assembles upon secretion and is crossed linked by lysyl oxidase to form the highly insoluble elastin polymer [13-16]. An example amino acid sequence of human tropoelastin sequence is shown in FIG. 2. The primary structure of tropoelastin comprises a series of alternating hydrophobic and the more highly conserved hydrophilic domains [13, 17]. As discussed earlier, elastin-like polypeptides (ELPs) are a special class of “smart” materials derived from the hydrophobic region of elastin that has been used for various biomedical applications including drug delivery and tissue engineering, as well as non-medical applications [9,11,18-23]. These ELP constructs are typically made up of more than 50 pentapeptide repeats in the form of homopolymer, diblock, and triblock copolymer blends [14, 24-27]. However, there are no identified reports on oligopeptides such as isolated peptides described herein (which are significantly smaller than the ELP constructs) being capable of self-assembling to form peptide nanostructures such as nanospheres described herein.

In accordance with some embodiments of one aspect described herein, a diverse library of hydrophobic peptides, e.g., 5-10 amino acids total in length, was designed. The hydrophobic peptides can self-assemble, e.g., in seconds, in aqueous media to generate a series of nanostructures (e.g., nanoparticles) with a capability to control the size of the nanostructures (e.g., nanoparticles) from nanometer to micrometer. These hydrophobic peptides can be used in various applications, e.g., for drug delivery and tissue engineering applications.

The library of amino acid sequence presented herein represents an entirely novel class of biocompatible biodegradable peptides that can spontaneously self-assemble into defined nanostructures but can also modulate at least one behavior of cells (e.g., but not limited to migration, viability, secretion, growth, apoptosis, differentiation, fate switching, and/or contractility). These novel peptide constructs can be useful for many applications, e.g., but not limited to, drug delivery, nanotherapeutics, diagnostics, and tissue engineering [30-32].

The self-assembly potential and the hydrophobic collapse of novel elastin-like oligopeptide sequences (e.g., 5-10 amino acids) can be identified by experiments, and/or computational simulations. For an experimental approach, a candidate peptide sequence can be synthesized as described herein, e.g., by solid-state peptide synthesis, and then subjected to various formulation buffers and/or processing conditions to evaluate its self-assembly potential. Characterization of any peptide nanostructures formed, e.g., size, shape, stability, and/or stimuli-responsiveness, can be performed using any methods known in the art or as described in the Examples below. For computational simulations, an algorithm for modeling a protein or peptide, such as Monte Carlo algorithms, can be used. Exemplary input modeling parameters for prediction of self-assembly can include, but are not limited to, hydrophobicity and charge state of the N- and C-termini.

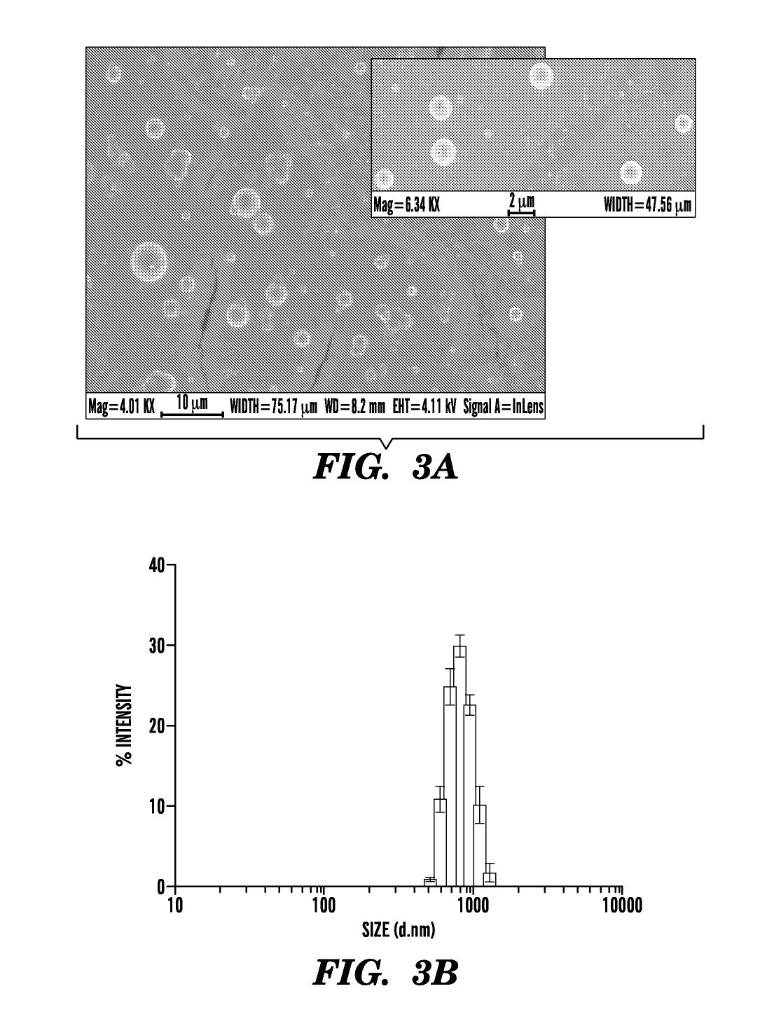

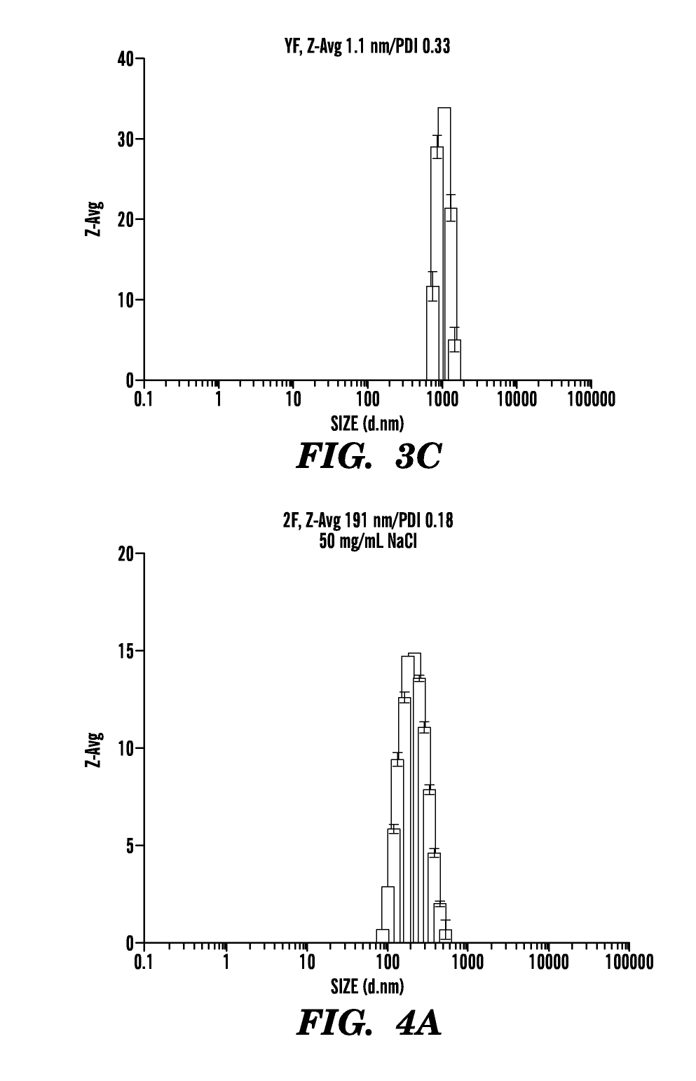

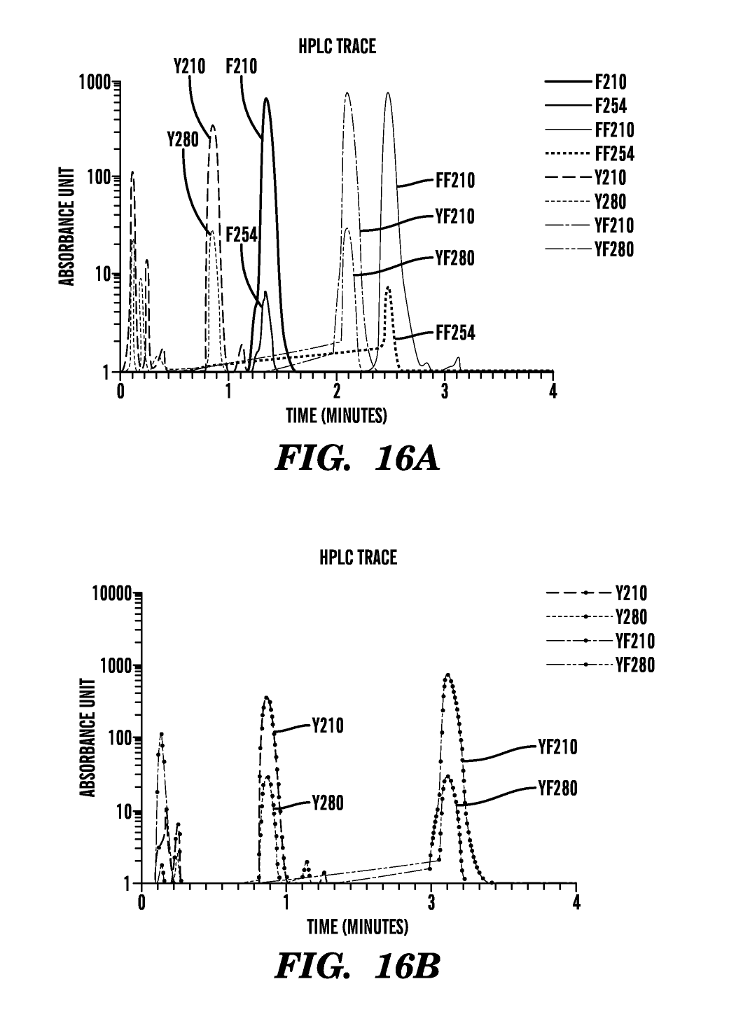

Exemplary self-assembling peptides comprising 5-10 amino acids are shown in Tables 1-2 and FIG. 1. The short peptide sequences having the general formula (X₁-X₂-X₃-X₄-X₃)ₙ, wherein X₁ through X₄ can be a combination of hydrophobic and/or aromatic amino acid (aa) residues. The 5 and 10 amino acids constructs were designed to mimic random hydrophobic domains in the human tropoelastin sequence as a means to test self-assembling properties of these mimetics (Tables 1-2). Each peptide in the Tables 1-2 was prepared, for example, by FMOC-based solid-phase peptide synthesis and all of the peptide sequences were verified for >90% purity before and directly following HPLC (FIGS. 16A-16B). The ability of these short hydrophobic peptide sequences to self-organize in aqueous media was then evaluated. As described in detail in the following Examples, the short peptides (as shown in Tables 1-2) formed a particulate suspension spontaneously within seconds in aqueous media. Scanning electron microscopic (SEM) and dynamic light scattering (DLS) studies showed that when the amino acid sequences in Tables 1-2 were each prepared at a concentration of about 50 mg/mL or 100 mg/mL in water, they self-assembled into spherical particles. For example, FIGS. 3A and 3B show nanoparticles self-assembled from FF peptides (in Table 1) and having an average hydrodynamic diameter of about 765 nm.

TABLE 1
Design of 10-amino acid
self-assembling peptide constructs

| | Sequence | |
|---|---|---|
| Entry | (Y₁-X₁-X₂-X₃-X₄-X₃-Y₂)n | NAME |
| 1 | VPGFGVPGFG | FF |
| 2 | VPGIGVPGLG | IL |
| 3 | VPGYGVPGFG | YF |
| 4 | VPGFGVPGYG | FY |
| 5 | VPGFGVPGYG | YY |
| 6 | VPGFGVPGWG | FW |
| 7 | VPGWGVPGFG | WF |

TABLE 2
Design of 5-amino acid
self-assembling peptide constructs

| | Sequence | |
|---|---|---|
| Entry | (Y₁-X₁-X₂-X₃-X₄-X₃-Y₂)n | NAME |
| 8 | VPGFG | F |
| 9 | VPGYG | Y |
| 10 | VPAYG | VPA |
| 11 | APGYG | APG |
| 12 | LPGPG | LPG |
| 13 | IPGYG | IPG |

### Example 2

### Exemplary Synthesis of Self-Assembling Peptides and Conditions for Formation of Nanostructures (Self-Assembly Conditions)

Self-assembling peptides (e.g., FF and YF peptides as shown in Table 1 and FIG. 1, each with a sequence of 10 amino acids in length) were each prepared by FMOC solid-phase peptide synthesis, for example, on Wang resin and cleaved from the resin with a solution mixture of trifluoroacetic acid/triisopropylsilane/water in a volume ratio of 9.5/2.5/2.5. The synthesized peptides were then purified by reversed phase HPLC. These peptide constructs were selected to represent bulky aliphatic and aromatic amino acid residues. Surprisingly, spontaneous self-assembly of these peptide constructs was induced by directly mixing about 1 mL of cold water (e.g., about 2° C. to about 4° C.) to pre-weighed peptide from about 2.5 mg to about 100 mg. The peptide concentration of the resulting mixture was about 2.5 mg/mL to about 100 mg/mL. The mixture was stirred at about 200 rpm to about 300 rpm for about 5 mins. The stirring speed can be varied to control size homogeneity. Scanning electron microscopy shows that the particles self-assembled from these peptide constructs (e.g., FF peptides) were substantially spherical as shown in FIG. 3A. Dynamic light scattering (DLS) analysis of these self-assembled particles indicated that these nanostructures formed from the FF peptides had an average hydrodynamic diameter in the 765 nm size range (FIG. 3B), while the ones formed from the YF peptides had an average hydrodynamic diameter in the 900 nm size range (FIG. 3C). Accordingly, the size of the nanostructures can be, at least in part, controlled by the sequence of the amino acid construct.

### Example 3

### Effects of Self-Assembly Conditions and Amino Acid Residue at the X₄ Position of the Amino Acid Construct on Stability and Size of Nanostructures

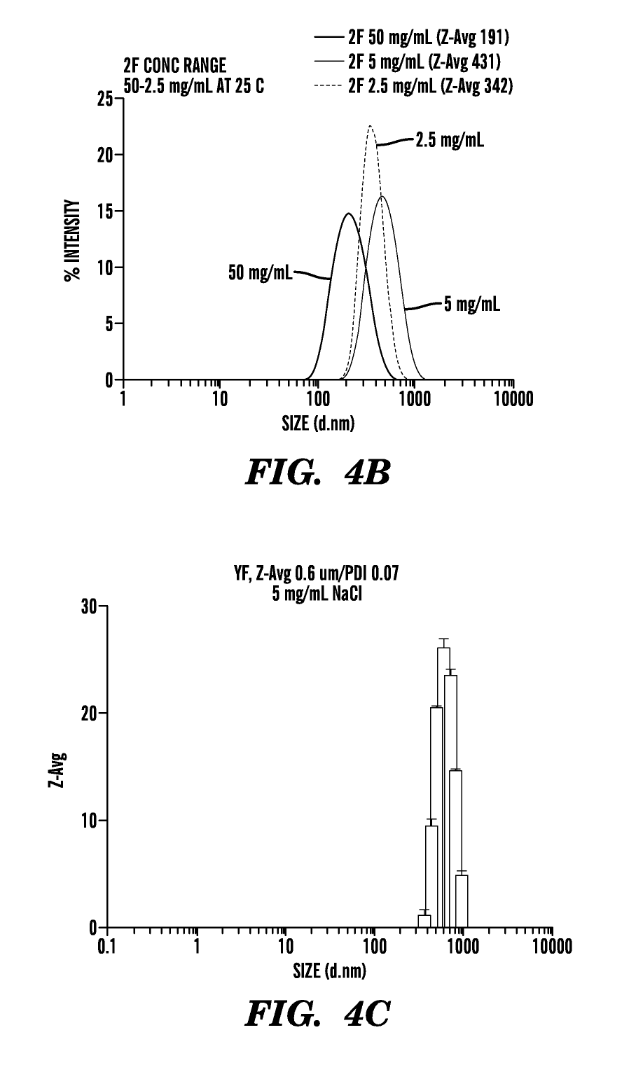

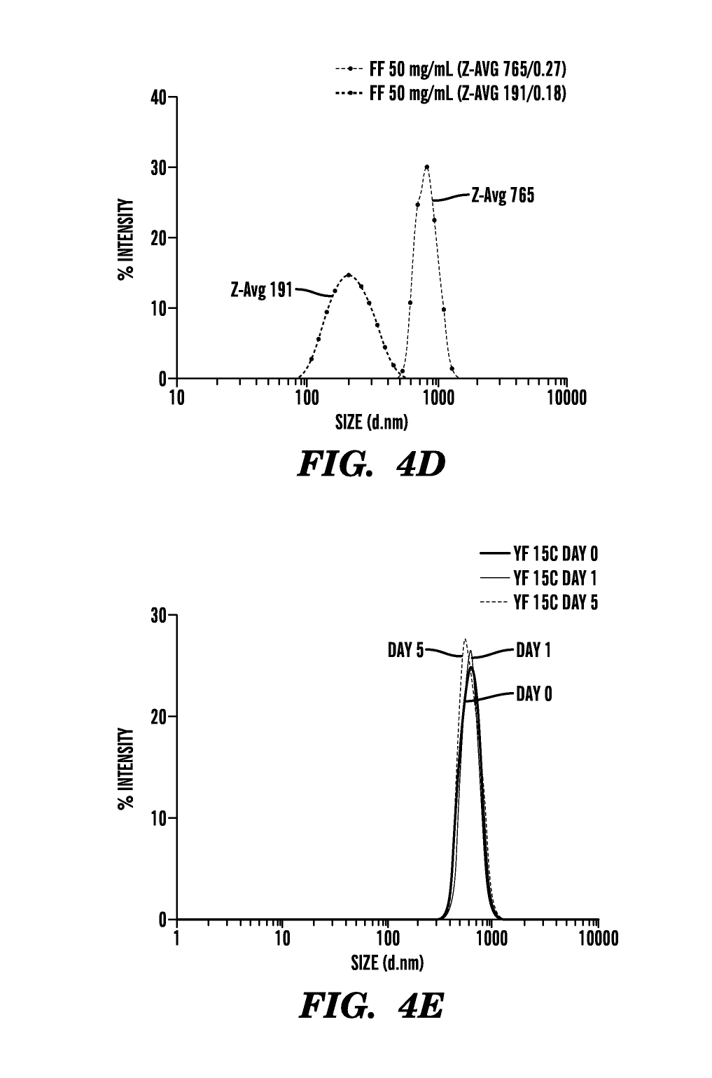

The self-assembled peptide nanoparticles formed (e.g., from FF or YF peptides) in cold water (cold deionized water) as shown in Example 2 were stable for about 2 hours before they eventually disaggregated as determined by DLS. To increase the stability of FF and YF, a solvent injection protocol was used. That is, the peptide constructs were dissolved in an organic solvent (e.g., but not limited to, DMSO, acetone, ethanol, dioxane, acetonitrile, methanol, and THF) at ˜150 mg/ml and a fixed volume was then injected in cold saline solution (e.g., but not limited to, ˜0.9% NaCl) while stirring. Different concentrations and/or types of salts could be used to prepare the saline solution, depending on the solubility of the peptide constructs in the respective solution. In some embodiments, any buffer solution such as PBS, acetate, succinate and citrate buffer could be used instead. The resulting particles size varied with peptide concentration from about 5 mg/ml to about 50 mg/ml with low polydispersities (FIGS. 4A-4B). By addition of 0.9% NaCl in the cold solution, the stability of the particles was increased from about 2 h to about 24 h as determined by DLS. Further, FIG. 4C indicates that a cold buffer solution (e.g., with addition of about 0.9% NaCl) can result in smaller self-assembled nanoparticles (e.g., YF nanoparticles) than the ones formed in cold deionized water (as shown in FIG. 3C; the peptide concentration was about 5 mg/mL). Similarly, as shown in FIG. 4D, the FF nanoparticles were smaller when they were formed in cold saline buffer (˜191 nm in diameter) than in deionized water (˜765 nm in diameter). Thus, in one embodiment, the average size of the nanoparticles can be controlled by simply varying the temperature of the formulation buffere, e.g., at room temperature or under cold ˜2-4° C. conditions.

In some embodiments, the nanoparticles can be formed by a process, which comprises dissolving an isolated peptide described herein (e.g., example peptides shown in Tables 1-2) in organic solvents such as DMSO at high concentration (e.g., about 400 mg/mL) and injecting a fixed volume of the dissolved peptides in cold saline (e.g., ˜0.9% sodium chloride solution) while stirring. The cold precipitation method (e.g., using a cold saline medium) can more efficiently induce peptide self-assembly. In some embodiments, the cold precipitation method (e.g., using a cold saline medium) can improve particle stability, e.g., from about 2 hours (e.g., when particles were formed by simply mixing the isolated peptides at a specified temperature) to about 24 hours (where the particles were formed by cold saline precipitation method described herein). In some embodiments, the cold precipitation method (e.g., using a cold saline medium) can also yield a more monodisperse or near-monodisperse particle distribution.

The ability to generate a wide range of particles sizes with low polydispersities can be desirable or advantageous in certain applications, e.g., but not limited to, nanotechnology and/or drug delivery. In some embodiments, the peptides described herein can form nanoparticles having a particle size with low polydispersity (e.g., with a polydispersity index of less than 0.5, less than 0.4, less than 0.3, less than 0.2, less than 0.1, or lower). In other embodiments, the peptides described herein can form nanoparticles having a particle size with a polydispersity index of about 0.5 or higher, e.g., at least about 0.5, at least about 0.6, at least about 0.7 or higher.

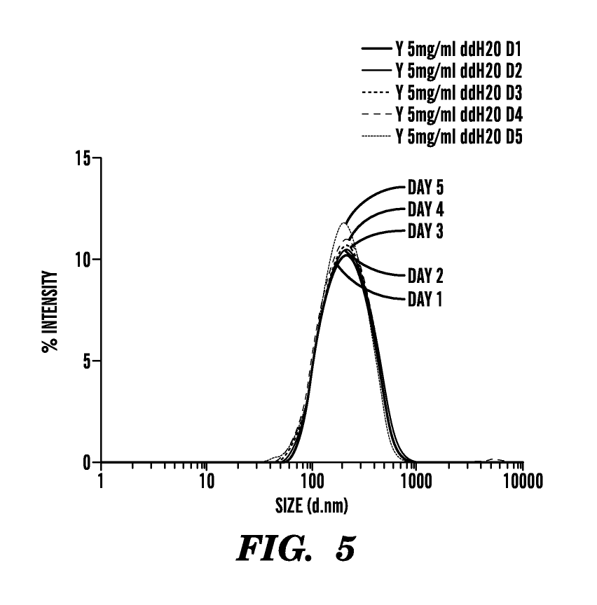

To further improve stability of nanoparticles, stability of the resulting particles was investigated as a function of amino acid residue at the X₄ position as indicated in Table 1 (entry 3-7) and X₄ position as indicated in FIG. 1. It was determined that the identity of the residue had an impact on the stability of the particle. For example, the particles formed from FY, YY, FW, and WF constructs did not show a significant increase in stability as compared to the ones formed from FF constructs, and were less stable as compared to the ones formed from the YF constructs (Data not shown). For example, as shown in FIG. 4E and FIG. 5, the particles self-assembled from the YF peptides or Y peptides can be stable for at least about 120 hours or longer when formed in the formulation buffer (e.g., ˜0.9% NaCl), e.g., using the cold precipitation method described earlier. Without wishing to be bound by theory, this increase in stability is likely, in part, due to the tyrosine residue at position X₄ that can be stabilized by the free amine at the N-terminus.

### Example 4

### Generation of Nanostructures with 5-Amino Acid Self-Assembling Peptide Sequences

There are no identified reports on peptides of 5 amino acids forming nanostructures, such as nanospheres. Accordingly, in order to determine if shorter peptide sequence can self-assemble into nanostructures, the selected 10-amino acid constructs in Table 1 were truncated to only 5 amino acid residues (as shown in Table 2) and particle size was measured by DLS. Remarkably, the shorter peptides (e.g., F and Y peptides in Table 2) self-assembled to form substantially spherical nanostructure with size similar to that observed for the 10-amino acid sequences. As shown in FIG. 5, peptide construct Y can be formulated (e.g., in ˜0.9% NaCl) to self-assemble into particles of similar size and comparable stability as compared to YF nanoparticles (e.g., formulated in ˜0.9% NaCl) (FIG. 5). Formulation buffers other than a salt buffer (e.g., ˜0.9% NaCl), including, but not limited to, PBS, acetate, succinate and citrate buffers, can also be used.

### Example 5

### Formation of Various Nanostructures Other than Spherical Particles or Nanospheres

The amino acid constructs described in Tables 1 and 2 are capable of forming different nanostructures, including nanofibers, nanorods, nanotubes and nanovesicles as a function of processing or formulation conditions. For example, as shown in FIGS. 6A-6D, the amino acid sequences (e.g., YF vs. Y vs. IL peptides as shown in Tables 1 and 2) and/or peptide concentration (e.g., between about 5 mg/mL and about 100 mg/mL) can influence types or forms of resulting nanostructures prepared under the same environmental conditions, e.g., same temperature and/or pH. It should be noted that the SEM preparation condition for the nanostructures shown in FIG. 6C was different from the others shown in FIGS. 6A, 6B and 6D, and included a series of ethanol/hexamethyldisilazane wash in place of freeze-drying and lyophilization.

In some embodiments, self-assembly into nanostructures such as nanofibers/nanorods can be more of a function of processing conditions than sequence specific. For example, keeping other conditions (e.g., temperatures, pH and amino acid sequence) constant, different nanostructures can be formed by varying concentrations of the self-assembling peptides of the same amino acid sequence. In some embodiments, the larger the difference in peptide concentration, the more well-defined the difference in nanostructure formed. For example, IL at a concentration greater than 300 mg/ml forms a fibrous network with very few visible particles (FIG. 6D) but forms a majority of particles at about or below 100 mg/mL in water.

The amino acid constructs (e.g., YF peptide and Y peptide as shown in Tables 1 and 2) are also temperature responsive and/or pH responsive. A range of nanostructures including tubular (FIG. 6A) and donut-like (FIG. 6B) morphologies were obtained when the initially-formed nanospheres were flash-frozen before lypohilization and imaged by SEM. FIG. 6E shows formation of a different FF nanostructure when the FF nanospheres as shown in FIG. 3A was frozen followed by lyophilization before SEM.

### Example 6

### Responses of Self-Assembling Peptides and Resulting Nanostructures to Environmental Stimuli

Self-assembling peptide constructs and the resulting nanostructures are responsive to environmental stimuli (FIG. 7A). For example, when the self-assembling constructs of the same peptide sequence were subjected to different self-assembly or processing conditions including pH and temperatures, the size of the resulting nanostructures varied. As shown in FIG. 7B, larger nanostructures (e.g., YF nanostructures) were formed at acidic pH (e.g., pH—1.5) than at basic pH (e.g., pH—10.5). Lower temperatures (e.g., —15° C.) resulted in larger nanostructures (e.g., FF nanostructures) than at higher temperatures (e.g., room temperature or higher) (FIG. 7C). However, for some self-assembling peptide constructs, larger nanostructures (e.g., YF nanostructures) were formed at higher temperatures than at lower temperatures (FIG. 7D). The peptide constructs can self-assemble in a neutral, acidic or basic buffer to form peptide nanostructures. As described earlier, the pH of the formulation buffer can influence the shape and/or size of the resulting nanostructures. While the DLS data presented only size information, the change in nanostructure size can be resulted from formation of nanostructures of different shapes (e.g., from spheres to nanorods) and/or a dimensional change of the nanostructure keeping the shape constant. For example, a sphere self-assembled from the peptide constructs can swell or shrink while remaining a sphere, and/or it can also change from a sphere to a nanorod.

While self-assembled nanostructures demonstrated stimuli-responsive behavior (e.g., they were subjected to different environmental conditions after they were already self-assembled), changes in nanostructures as a function of processing conditions were also determined. For example, nanostructure size can be varied as a function of pH and/or temperature of the formulation buffer during self-assembly.

Self-assembling peptide constructs are also responsive to formulation conditions including peptide concentration and modification of the peptide construct. For example, FIG. 7E indicates that keeping other conditions constant, higher peptide concentration during a self-assembly process can result in larger nanostructures. In some embodiments, the form/shape of nanostructures can change (e.g., from spheres to rods) when all other processing conditions remain the same but the relative peptide concentrations are significantly higher than or at some critical levels. The critical concentrations of each peptide construct can vary depending on the amino acid sequence of the construct. For example, peptide construct IL at a concentration of about 300 mg/mL can form a different nanostructure as compared to the same peptide construct at a concentration of about 100 mg/mL (Data not shown). FIG. 7F shows the difference between YF-only particles and YF particles encapsulating a protein, e.g., serum albumin (the serum albumin can be modified, e.g., with PEG-FITC for imaging purposes). The human serum albumin was added to the formulation buffer during self-assembly. Without wishing to be limited, any active agent as described herein can be added to the formulation buffer during self-assembly to generate peptide nanostructures encapsulating the active agent. In one embodiment, a therapeutic agent, e.g., doxorubicin, can be added to the formulation buffer during self-assembly to generate self-assembled particles encapsulating the therapeutic agent.

### Example 7

### Exemplary Modifications of Self-Assembling Peptides

Self-assembling peptides can be modified for conjugation to various agents or substrates, such as polymer, nanoparticles, a hydrogel, a protein, an aptamer, a detection label, a therapeutic agent, depending on users' applications such as diagnostic applications, drug delivery, biosensors, and tissue engineering.

For example, the FF, IL and VK peptides were conjugated to nanoparticles (such as gold nanoparticles), e.g., optionally via a coupling molecule. For example, as shown in FIG. 13A, the peptide construct (e.g., FF, IL, or VK constructs) can be conjugated to a gold nanoparticle (AuNP) via a linker (e.g., but not limited to, Trityl-S-dPEG®4-acid or alpha lipoic acid). In one embodiment, the peptide-AuNP constructs were prepared by first modifying peptide constructs (e.g., FF, IL or VK constructs) with one or more sulphur-containing organic compounds such as Trityl-S-dPEG®4 or αLipoic acid, while each peptide was still on a Wang resin under standard solid phase peptide chemistry. Cleavage from the resin and HPLC purification was carried out as described earlier and the sulfhydryl/thiol-based peptides were added directly to AuNPs and allowed to bind to the AuNPs through the sulphur functional group for up to 16 h or overnight. Other art-recognized methods, e.g., described in Lemieux et al. (2010) Chem. Commun., 46: 3071-3073, for conjugating one or more peptide constructs to a nanoparticle can also be used herein. For example, the peptide constructs can be prepared by a modified version of standard Fmoc-based solid-phase peptide synthesis techniques. When the peptide construct is still on the resin, the terminal valine of the construct can be deprotected and coupled to 3-mercapto-propionic acid in the presence of HOBt and DIPCDI. The resulting peptide can then be cleaved from the resin, resulting in a free carboxylic acid at one end and a thiol at the other end. A ligand-exchange reaction from ligand-capped nanoparticles (e.g., 4-(N,N-dimethylamino)pyridine (DMAP)-capped gold nanoparticles) can be used for preparation of peptide-conjugated nanoparticles (e.g., gold nanoparticles). For example, the addition of a stoichiometric quantity of the thiol-peptide construct to an aqueous solution of DMAP-capped gold nanoparticles can be prepared, for example, according to the procedure described in Gittins and Caruso (2001) Angew. Chem., Int. Ed. 40: 3001-3004; and Gandubert and Lennox (2005) Langmuir 21: 6532-6539, for a ligand-exchange reaction to take place at room temperature and under ambient atomosphere over a period of time (e.g., at least about 12 hours or more).

In order to determine if the self-assembling peptides remain responsive to an environmental stimulus after conjugation to a substrate (e.g., gold nanoparticles (AuNPs)), the self-assembling peptides conjugated to a gold nanoparticle were subjected to different pHs and/or temperatures. For example, the peptide constructs (e.g., FF constructs) were able to induce reversible pH- and/or temperature-responsive behavior in peptide-conjugated gold nanoparticles. In one embodiment, as shown in FIG. 13B, the FF-modified gold nanoparticles aggregated to form larger nanostructures (e.g., ˜500-600 nm) when the pH was decreased (e.g., from pH˜6 to pH˜4).

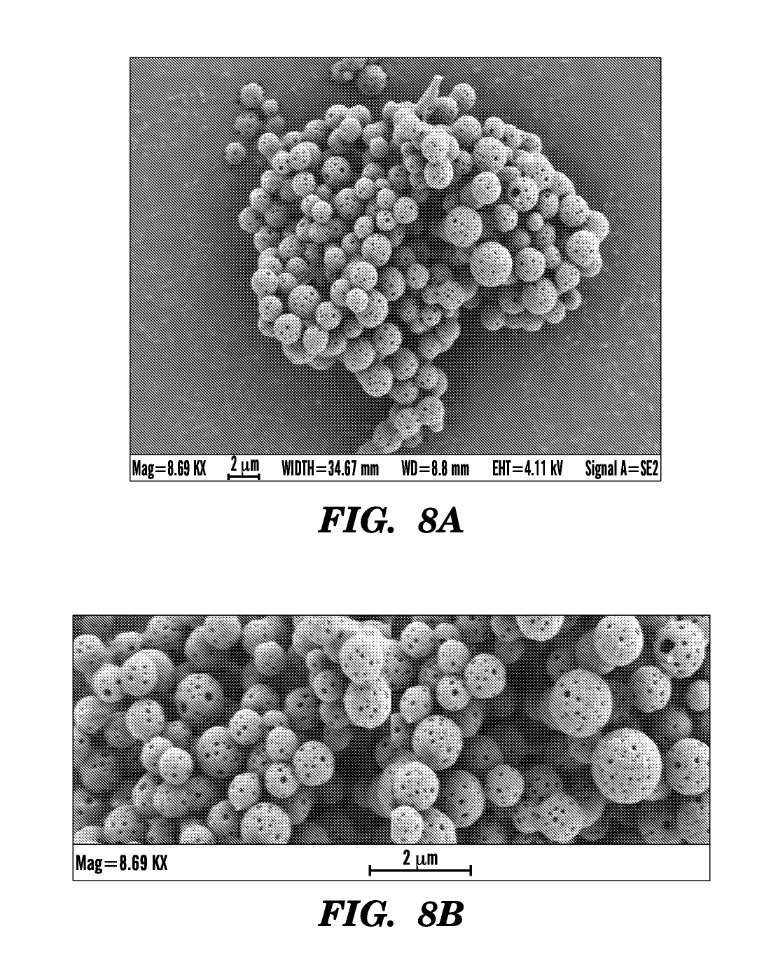

In some embodiments, the self-assembling peptides can be conjugated to a polymer. For example, as shown in FIGS. 8A-8B, the FF peptides conjugated to PLGA formed porous nanoparticles by solvent precipitation. In one embodiment, PLGA-FF (PLGA 50:50, MW ˜17 kDa; FF MW ˜1 kDa) constructs were prepared by standard solid-phase peptide chemistry with C-terminus of FF peptide covalently immobilized on a Wang resin and PLGA coupled to the N-terminus with coupling agents such as 1-hydroxybenzotriazole (HOBT)/diisopropylcarbodiimide (DIC). The reaction upon completion was cleaved from the resin with a solution mixture of trifluoroacetic acid/triisopropylsilane/water in a volume ratio of 9.5/2.5/2.5. The pure product was isolated by precipitation, e.g., in cold ether. PLGA-FF constructs were dissolved in DMSO and dialyzed in water. The PLGA-FF peptides are temperature responsive.

### Example 8

### Exemplary Applications of Self-Assembled Nanostructures

The self-assembling peptides (e.g., 5-10 amino acid constructs) can self-assemble into defined nanostructures, including nanospheres, nanocapsules, and nanofibers. When used alone or when integrated into larger three-dimensional (3D) porous scaffolds, these nanomaterials can modulate the mechanical property of the local environment to alter tissue mechanics (e.g., in fibrosis or cancer), deliver a wide range of drugs from small molecule drugs to biologics for therapeutic applications, regulate cellular activities (e.g., mechanically control stem cell fate switching, or chemically inhibit enzyme activities), using a range of external triggers (e.g., temperature, pH, etc.).

For example, the peptide constructs (e.g., VK, FF, YF and Y peptide as shown in Tables 1 and 2) are used to induce temperature-dependent gel formation in protein (e.g., human serum albumin) and biopolymers (e.g., hyaluronic acid). In such application, the N-terminus of the peptide constructs can be modified with a maleimide function group to induce gel formation. In one embodiment, FF-maleimide was coupled to serum albumin and induced gel formation. The resulting gel can be used as a temperature-sensitive drug delivery system. Further, the gel stiffness can be modulated by varying temperatures, which can be desirable for tissue engineering scaffolds.

Without wishing to be limited, self-assembled nanostructures can be preformed from the peptide constructs described herein before they are dispersed in a gel, hydrogel or a polymer. For example, as shown in FIG. 9, the HA hydrogel stiffness can be modulated by temperatures through impregnation with FF nanoparticles. Increasing temperatures from 4° C. to body temperature (e.g., 37° C.) decreased the stiffness of the HA hydrogel, as evidenced by a lower modulus determined by dynamic mechanical analysis using a frequency sweep.

To demonstrate the utility of these short peptides as good candidate for drug carriers, peptide constructs (e.g., YF and Y peptides with longer stability) were selected to determine the potential for encapsulating one or more model agents. Nile Red (NR) a hydrophobic dye and Calcein, hydrophilic dyes were used as model agents for hydrophobic and hydrophilic drugs or molecules, respectively. For example, YF was dissolved in DMSO to make a stock concentration of about 380 mg/mL. Stock solutions of Nile red and Calcein dyes were each prepared in DMSO at about 3 mg/ml and about 20 mg/mL, respectively. A mixture of YF (about 380 mg/ml) and at least one model agent (e.g., Nile red (0.28 mg/mL) and/or Calcein (1.8 mg/mL)) was added to a formulation buffer, e.g., cold saline (e.g., about 0.3 mL) and mixed, e.g., by manual pipetting. The final ratios of peptide to dyes were ˜25 mg/ml to ˜0.02 mg/ml (YF:NR) and ˜25 mg/mL to ˜0.124 mg/mL (YF:calcein). In some embodiments, about 20-30% of each dye was encapsulated within self-assembling YF nanoparticles.

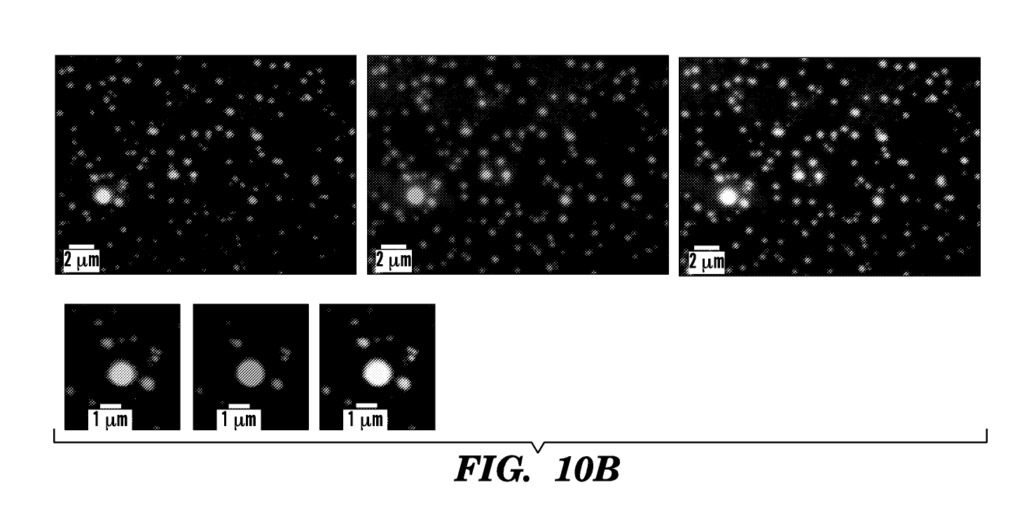

As shown in FIGS. 10A-10B, these peptide constructs (e.g., YF and Y peptides) are able to efficiently encapsulate both a hydrophobic agent (e.g., Nile Red) and a hydrophilic agent (e.g., Calcein) as observed by fluorescent microscopy. While the peptide constructs described herein can behave as amphiphilic constructs, the peptides that self-assemble into nanostructures are generally hydrophobic constructs, and thus they are not classical amphiphilic constructs. However, the hydrophobic peptide constructs described herein can have sufficient functional groups such as free N- and C-termini and the amide backbone for capturing hydrophilic materials or compounds and the hydrophobic side chains for capturing hydrophobic materials or compounds. Notably, the highest concentrations of dye molecules are observed in the inner core of the particles with higher fluorescent intensities.

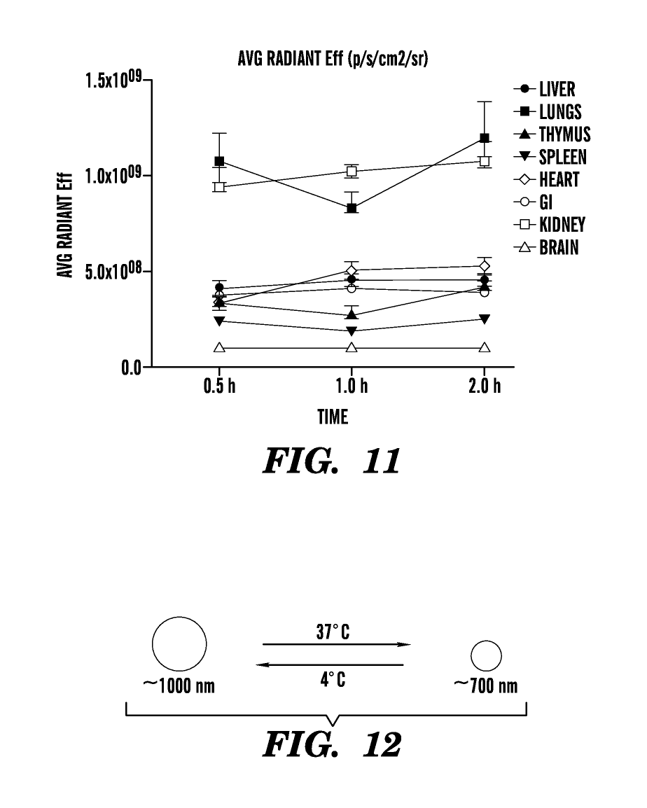

Chemical and physical properties of the resulting nanoparticles, including size, surface charge, and surface chemistry, are important factors that determine their pharmacodynamics and biodistribution, which define their efficacy to deliver an agent and toxicity. Accordingly, it was next sought to determine tissue distribution of these resulting nanoparticles. Specifically, Alexa 750 dye was encapsulated in YF nanoparticles and administered to mice by tail vein injection. At intervals of 0.5, 1.0, and 2.0 hours, the animals were euthanized, then dissected. As shown in FIG. 11, high levels of fluorescence from the nanoparticles were detected and maintained for at least 2 hours, indicating its stability in vivo. Further, greater deposition of the nanoparticles in the lungs was observed, indicating that these nanoparticles can be used for targeting local delivery to the lungs as well as systemic delivery, e.g., by inhalation. These nanoparticles can potentially eliminate the need for expensive spraying approach in aerosol delivery. It should be also noted that these nanoparticles can cross the blood-brain-barrier and deposit in the brain, as evidenced by fluorescence in the brain of the mice; thus, these nanoparticles can be desirable to encapsulate and deliver a therapeutic agent that would otherwise not able to cross the blood-brain-barrier by itself.

### Example 9

### Effects of Conservative Substitutions on Size Distribution of Self-Assembled Peptide Nanostructures

In accordance with some embodiments described herein, the amino acid sequence of the isolated peptide can include one or more (e.g., 1, 2, 3, 4, or more) conservative substitutions. The conservative substitution can occur at any residue in the amino acid sequence. To assess the effects of conservative substitutions on self-assembled peptide nanostructures, one amino acid residue (e.g., X₁ or X₃) in the amino acid sequence of the isolated peptide was replaced by a conservative substitution. For example, as shown in FIG. 14, in some embodiments, valine (Val) at the X₁ position was replaced by alanine (Ala), leucine (Leu), isoleucine (Ile); while in some embodiments, glycine (Gly) was replaced by alanine (Ala).

Each peptide was dissolved in an organic solvent (e.g., but not limited to, DMSO) at about 380 mg/mL and injected in cold saline solution at about 2-4° C., resulting in a final peptide concentration of about 25 mg/mL. As shown in FIG. 14, a conservative substitution present in the peptide construct can generate peptide nanostructures (e.g., peptide nanoparticles) of different dimensions and/or size distributions. For example, nanoparticles generated from IPGYG peptides were more monodisperse than the ones generated from the other peptide constructs.

### Example 10

### Effects of Peptide Constructs and/or Peptide Nanostructures on Cell Viability

The viability of cells incubated with various concentrations of peptide constructs and/or resulting peptide nanoparticles were evaluated. For example, murine breast cancer cells (e.g., 4T1 and M6 cells) were cultured with Y peptides (and/or resulting Y peptide nanoparticles) or YF peptides (and/or resulting YF peptide nanoparticles) and it was found that greater than 80% of the cells were viable after at least about 24 hours or longer (e.g., at least about 1 week or longer) in culture (Data not shown).

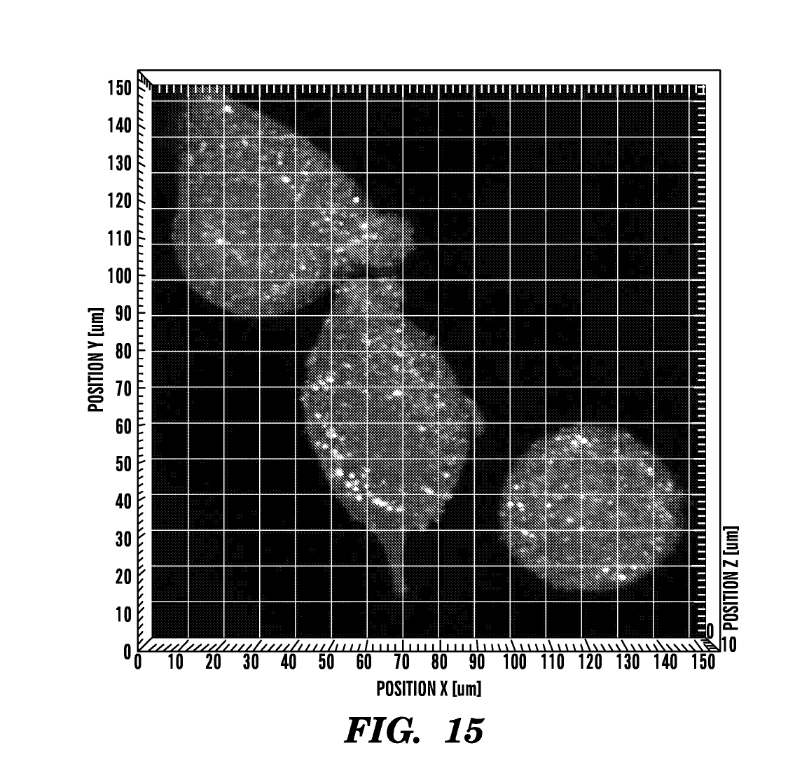

Furthermore, the ability of these peptide constructs to be taken up by cells was also evaluated. The cells were incubated with the peptide nanoparticles described herein at room temperature. As shown in FIG. 15, the peptide nanoparticles were taken up into the intracellular compartment of NMuMg normal mouse mammary gland cells at approximately ˜500 nanoparticles/cell.

The Examples described herein show that the novel class of short, self-assembling peptides described herein can form nanostructures that can be tuned to various sizes from nanometer to micrometer scale with a desired degree of polydispersity. For example, in some embodiments, the short, self-assembling peptides described herein can form nanostructures that can be tuned to various sizes with monodisperse or near-monodisperse size distribution. In some embodiments, the stability of the peptide nanoparticles described herein can be also tunable by varying, e.g., but not limited to amino acid sequence of the peptides, self-assembly condition (e.g., temperature, and/or pH), and/or formulation mediu. In some embodiments, the peptide nanoparticles can be used to encapsulate and/or stabilize any agent of interest, e.g., but not limited to, hydrophobic molecules, hydrophilic molecules, proteins, nucleic acid molecules (e.g., DNA, and RNA including, e.g., mRNA, tRNA, RNAi, siRNA, microRNA, or any other art-recognized RNA or RNA-like molecules), nucleotides, biologics, drugs or therapeutic agents, or any combinations thereof. In some embodiments, the peptide nanoparticles can be used to encapsulate a labile agent and stabilize the activity of the labile agent during storage and/or transportation, and/or upon administration of the labile agent to a subject.

### Exemplary Materials and Methods Used in Examples 1-10

Materials.

High grade reagents and anhydrous solvents were purchased and used without any further purification unless indicated otherwise. All peptide sequences shown in Tables 1-2 were synthesized by solid phase peptide chemistry using Fmoc Chemistry. The peptide equences were purified by HPLC using a C18 5 μm 120 A 4.6*150 mm column in 0.1% TFA/H2O (buffer A) and 0.09% TFA in 80% ACN/20% H₂O (buffer B).

Nanoparticle Formulation.

Each peptide was dissolved in distilled deionized water at varying concentrations, which can, in part, control particle size. Mixtures of ˜80 mg/mL, ˜50 mg/mL, and ˜20 mg/mL peptide concentration were each stirred vigorously (e.g., using a magnetic stirrer) for about 10 mins at room temperature or at about 4° C. Nanoparticles were measured, e.g., by dynamic light scattering (DLS), to be in the range from about 50 nm to about 2 μm.

In addition or alternatively, particle size was controlled by using a solvent precipitation method. For example, a stock solution of the peptides described herein at a high concentration (e.g., about 400 mg/mL) was prepared in an organic solvent (e.g., DMSO) and then slowly added to distilled deionized water or a buffered solution (e.g., PBS) while vigorously stirring.

Dynamic Light Scattering (DLS).

A zeta particle size analyzer (Malvern instruments, UK) operating with a HeNe laser, and a 173° back scattering detector was used to determine the size distribution of the nanoparticles. Samples were prepared at 80 mg/mL, 50 mg/mL, and 20 mg/mL in water and measured directly by dynamic light scattering measurement (n=3 per condition). Malvern instrument software or Microsoft Excel was used to analyze the acquired data.

Transmission Electron Microscopy (TEM).

A JEOL 1400 TEM microscope (JEOL, Peabody, Mass., USA) was used to characterize the morphology of the peptide nanoparticles. About 5 μL of nanoparticle solutions was added onto Formvar 400 mesh copper grids. After ˜5 minutes, the excess solution was wicked by filter paper and the sample was washed with water. The sample was then stained with 0.75% uranyl formate (Polysciences Inc, PA, USA) and air dried for about 5 mins prior to imaging.

Cell Viability and Proliferation.

To assess effects of the peptides and/or peptide nanostructures described herein on cell viability, cells were grown to confluence in gelatin 96-well plates, following which they were either left untreated or treated with blank or one or more embodiments of the peptide nanoparticles (˜25 mg/ml) for about 18 hours. CellTiter-Blue® reagent was then added to each well and, following 4 hour incubation at ˜37° C., the fluorescence signal was measured using a fluorescence multiwell plate reader (Victor3™, PerkinElmer, Mass., USA). All fluorescent intensity measurements were then normalized with respect to the untreated 4T1 mouse mammary carcinoma cells.

Encapsulation of Hydrophobic and Hydrophilic Dyes in Peptide Nanostructures.

The peptide nanostructures (e.g., peptide nanoparticles (NP)) were visualized by fluorescence microscopy using the hydrophobic dye, Nile Red, which has a strong emission at ˜525 nm when present in a lipid-rich environment and excited at ˜485 nm, or the hydrophilic dyes, calcein (excitation/emission 495 nm/515 nm) and FITC-Dextran (excitation/emission 495 nm/521 nm). For example, to encapsulate a hydrophobic and/or hydrophilic dye in peptide nanostructure, one or more embodiments of the peptides described herein (e.g., ˜25 mg/mL) were dissolved in distilled deionized water containing about 0.5 mg/mL Nile Red and/or 2.0 mg/mL calcein, thus forming peptide nanostructures with the dye of interest encapsulated therein. An aliquot of ˜10 μL solution was then added to a glass cover slip for visualization using fluorescence microscopy (TIRF DM1600).

Cell Encapsulation Study.

To evaluate the ability of one or more embodiments of the peptides described herein to be taken up by cells and thus the utility for intracellular delivery of drugs, Alexa 647 (A647) dye were encapsulated in YF and Y peptide nanostructures as described herein and incubated with various cell types, for example, using the following example protocol as described below.

Two self-assembling peptides of interest, Y and YF, were prepared at concentrations of about 388 mg/ml in an organic solvent (e.g., DMSO). To formulate the dye-loaded particles, an aliquot of the peptide solution (˜20 μL) was added to the A647 dye solution prepared in DMSO (e.g., ˜0.4 μL containing A647 dye at ˜2 mg/mL). The peptide-dye solution was then gently mixed (e.g., with a pipette) and allowed to sit at room temperature, e.g., for about 5 minutes. It was then transferred into a cold buffered solution (e.g., about 300 μL of cold PBS) and gently mixed. DLS measurements of particle size can be taken from these samples. An aliquot of the peptide-dye solution was then added to an appropriate cell culture medium (e.g., High Glucose DMEM, F12K depending on cell types) to prepare the solution delivered to the cells.

Cell uptake of peptide nanoparticles was assessed in the following cell lines: A549, 3T3, M6, NMuMg, and EpH4. The cells were seeded at a density of about 80,000 cells/ml medium onto 10 mm MatTek dishes at a volume of about 1 mL. Upon incubation at ˜37° C. for about 72 hours, e.g., to achieve semi-confluence for ease of imaging, the cell medium was replaced with the peptide-dosed medium for incubation at ˜37° C., e.g., for about 1-3 hours. Following the incubation, the cells were washed twice with a buffered solution (e.g., PBS) to remove any peptide nanoparticles on the outside surface of the cells in the MatTek dishes and the cells were thex fixed with a 4% paraformaldehyde solution. The cells were subsequently stained with 1×HCS CellMask Green/Blue and 1×HCS NuclearMask Blue and mounted in Prolong Gold for fluorescence imaging, e.g., on a Leica SP5× MP Inverted Confocal Microscope.

Statistical Analysis.

All data are obtained from multiple replicates, as indicated in the respective procedures, and expressed as mean±SEM. Statistical significance was determined using analysis of variance (ANOVA; InStat®, GraphPad Software Inc.). Results were considered significant if p<0.01.

### REFERENCES

1 Henry, C. M. DRUG DELIVERY. Chemical & Engineering News Archive 80, 39-47, doi:10.1021/cen-v080n034.p039 (2002).
2 Sallach, R. E. et al. Micelle density regulated by a reversible switch of protein secondary structure. Journal of the American Chemical Society 128, 12014-12019, doi:10.1021/ja0638509 (2006).
3 Jordan, S. W. et al. The effect of a recombinant elastin-mimetic coating of an ePTFE prosthesis on acute thrombogenicity in a baboon arteriovenous shunt. Biomaterials 28, 1191-1197, doi:10.1016/j.biomaterials.2006.09.048 (2007).
4 Caves, J. M. & Chaikof, E. L. The evolving impact of microfabrication and nanotechnology on stent design. Journal of vascular surgery 44, 1363-1368, doi:10.1016/j.jvs.2006.08.046 (2006).
5 Estella-Hermoso de Mendoza, A., Campanero, M. A., Mollinedo, F. & Blanco-Prieto, M. J. Lipid nanomedicines for anticancer drug therapy. Journal of biomedical nanotechnology 5, 323-343 (2009).
6 Farrell, D., Ptak, K., Panaro, N. J. & Grodzinski, P. Nanotechnology-based cancer therapeutics—promise and challenge—lessons learned through the NCI Alliance for Nanotechnology in Cancer. Pharmaceutical research 28, 273-278, doi:10.1007/s11095-010-0214-7 (2011).
7 Garcia, A. et al. Microfabricated engineered particle systems for respiratory drug delivery and other pharmaceutical applications. Journal of drug delivery 2012, 941243, doi:10.1155/2012/941243 (2012).
8 Gebauer, M. & Skerra, A. Engineered protein scaffolds as next-generation antibody therapeutics. Current opinion in chemical biology 13, 245-255, doi:10.1016/j.cbpa.2009.04.627 (2009).
9 Hubbell, J. A. & Chilkoti, A. Chemistry. Nanomaterials for drug delivery. Science (New York, N.Y.) 337, 303-305, doi:10.1126/science.1219657 (2012).
10 Loo, Y., Zhang, S. & Hauser, C. A. From short peptides to nanofibers to macromolecular assemblies in biomedicine. Biotechnology advances 30, 593-603, doi:10.1016/j.biotechadv.2011.10.004 (2012).
11 Branco, M. C., Sigano, D. M. & Schneider, J. P. Materials from peptide assembly: towards the treatment of cancer and transmittable disease. Current opinion in chemical biology 15, 427-434, doi:10.1016/j.cbpa.2011.03.021 (2011).
12 Zhu, J. & Marchant, R. E. Design properties of hydrogel tissue-engineering scaffolds. Expert review of medical devices 8, 607-626, doi:10.1586/erd.11.27 (2011).
13 Vrhovski, B. & Weiss, A. S. Biochemistry of tropoelastin. European journal of biochemistry/FEBS 258, 1-18 (1998).
14 Almine, J. F. et al. Elastin-based materials. Chemical Society reviews 39, 3371-3379, doi:10.1039/b919452p (2010).
15 Akagawa, M. & Suyama, K. Mechanism of formation of elastin crosslinks. Connective tissue research 41, 131-141 (2000).
16 Reiser, K., McCormick, R. J. & Rucker, R. B. Enzymatic and nonenzymatic cross-linking of collagen and elastin. FASEB journal: official publication of the Federation of American Societies for Experimental Biology 6, 2439-2449 (1992).
17 Mithieux, S. M. & Weiss, A. S. Elastin. Advances in protein chemistry 70, 437-461, doi:10.1016/50065-3233(05)70013-9 (2005).
18 Chen, T. H., Bae, Y. & Furgeson, D. Y. Intelligent biosynthetic nanobiomaterials (IBNs) for hyperthermic gene delivery. Pharmaceutical research 25, 683-691, doi:10.1007/s11095-007-9382-5 (2008).
19 Wu, Y., MacKay, J. A., McDaniel, J. R., Chilkoti, A. & Clark, R. L. Fabrication of elastin-like polypeptide nanoparticles for drug delivery by electrospraying. Biomacromolecules 10, 19-24, doi:10.1021/bm801033f (2009).
20 MacEwan, S. R. & Chilkoti, A. Elastin-like polypeptides: biomedical applications of tunable biopolymers. Biopolymers 94, 60-77, doi:10.1002/bip.21327 (2010).
21 Hassouneh, W., Christensen, T. & Chilkoti, A. Elastin-like polypeptides as a purification tag for recombinant proteins. Current protocols in protein science/editorial board, John E. Coligan . . . [et al.] Chapter 6, Unit 6 11, doi:10.1002/0471140864.ps0611s61 (2010).
22 Aluri, S., Pastuszka, M. K., Moses, A. S. & MacKay, J. A. Elastin-like peptide amphiphiles form nanofibers with tunable length. Biomacromolecules 13, 2645-2654, doi:10.1021/bm300472y (2012).
23 Machado, R., Bessa, P. C., Reis, R. L., Rodriguez-Cabello, J. C. & Casal, M. Elastin-based nanoparticles for delivery of bone morphogenetic proteins. Methods Mol Biol 906, 353-363, doi:10.1007/978-1-61779-953-2_{—}29 (2012).
24 Osborne, J. L., Farmer, R. & Woodhouse, K. A. Self-assembled elastin-like polypeptide particles. Acta biomaterialia 4, 49-57, doi:10.1016/j.actbio.2007.07.007 (2008).
25 Fujita, Y., Mie, M. & Kobatake, E. Construction of nanoscale protein particle using temperature-sensitive elastin-like peptide and polyaspartic acid chain. Biomaterials 30, 3450-3457, doi:10.1016/j.biomaterials.2009.03.012 (2009).
26 Kim, W., Thevenot, J., Ibarboure, E., Lecommandoux, S. & Chaikof, E. L. Self-assembly of thermally responsive amphiphilic diblock copolypeptides into spherical micellar nanoparticles. Angew Chem Int Ed Engl 49, 4257-4260, doi:10.1002/anie.201001356 (2010).
27 Verdine, G. L. & Hilinski, G. J. Stapled peptides for intracellular drug targets. Methods in enzymology 503, 3-33, doi:10.1016/b978-0-12-396962-0.00001-x (2012).
28 Wise, S. G., Mithieux, S. M. & Weiss, A. S. Engineered tropoelastin and elastin-based biomaterials. Advances in protein chemistry and structural biology 78, 1-24, doi:10.1016/S1876-1623(08)78001-5 (2009).
29 Wu, X., Levenston, M. E. & Chaikof, E. L. A constitutive model for protein-based materials. Biomaterials 27, 5315-5325, doi:10.1016/j.biomaterials.2006.06.003 (2006).
30 Sugahara, K. N. et al. Coadministration of a tumor-penetrating peptide enhances the efficacy of cancer drugs. Science (New York, N.Y.) 328, 1031-1035, doi:10.1126/science.1183057 (2010).
31 S Zhang, X. Z., L Spirio. in Scaffolding in Tissue Engineering (ed Ma and Elisseeff) 217-238 (CRC Press, 2005).
32 Annabi, N., Mithieux, S. M., Weiss, A. S. & Dehghani, F. Cross-linked open-pore elastic hydrogels based on tropoelastin, elastin and high pressure CO2. Biomaterials 31, 1655-1665, doi:10.1016/j.biomaterials.2009.11.051 (2010).

Content of all patents and other publications identified herein is expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

We claim:

1.-145. (canceled)

146. A composition comprising an aggregate of self-assembling peptides, wherein the self-assembling peptides each consists essentially of:
an amino acid sequence of (Y₁-X₁-X₂-X₃-X₄-X₃-Y₂)ₙ; and
at least one entity conjugated to the amino acid sequence,
wherein:
X₁ is valine (Val) or a conservative substitution thereof;
X₂ is proline (Pro) or a conservative substitution thereof;
X₃ is glycine (Gly) or a conservative substitution thereof;
X₄ in each nth unit is independently an amino acid residue, wherein when n is 4, at least one X₄ is not valine;
Y₁ and Y₂ are each independently a linker, wherein the linker is selected from a bond, one amino acid residue or a group of amino acid residues, wherein when Y₁ and Y₂ are each independently one amino acid residue or a group of amino acid residues, the combined amino acid sequences of Y₁ and Y₂ does not comprise a sequence of (VPGX₄G);
n is an integer from 1 to 10; and
the entity is selected from a group consisting of —H, —OH, a chemical functional group, a ligand, an active agent, a therapeutic agent, a binding molecule, a coupling molecule, a labeling agent, a peptide-modifying molecule, and a solid substrate, wherein when the amino acid sequence is a repeated sequence of (VPGVG), the solid substrate is not a biodegradable non-amino acid moiety.

147. The composition of claim 146, wherein the amino acid sequence is (Y₁-Val-Pro-Gly-X₄-Gly-Y₂)ₙ, wherein each amino acid residue is independently a D-amino acid or a L-amino acid.

148. The composition of claim 146, wherein the X₄ is selected from the group consisting of phenylalanine (Phe), isoleucine (Ile), leucine (Leu), tyrosine (Tyr), tryptophan (Trp), valine (Val), lysine (Lys), histidine (His), methionine (Met), a non-standard amino acid, a side-chain modified amino acid, and a derivative thereof.

149. The composition of claim 146, wherein n is an integer of 1, 2 or 3.

150. The composition of claim 146, wherein the amino acid sequence is selected from the group consisting of

| | |
|---|---|
| a. | Val-Pro-Gly-Phe-Gly-Val-Pro-Gly-Phe-Gly; |
| b. | Val-Pro-Gly-Ile-Gly-Val-Pro-Gly-Leu-Gly; |
| c. | Val-Pro-Gly-Tyr-Gly-Val-Pro-Gly-Phe-Gly; |
| d. | Val-Pro-Gly-Phe-Gly-Val-Pro-Gly-Tyr-Gly; |
| e. | Val-Pro-Gly-Trp-Gly-Val-Pro-Gly-Phe-Gly; |
| f. | Val-Pro-Gly-Phe-Gly-Val-Pro-Gly-Trp-Gly; |
| g. | Val-Pro-Gly-Tyr-Gly-Val-Pro-Gly-Tyr-Gly; |
| h. | Val-Pro-Gly-Trp-Gly-Val-Pro-Gly-Trp-Gly; |
| i. | Val-Pro-Gly-Phe-Gly; |
| j. | Val-Pro-Gly-Tyr-Gly; |
| k. | Val-Pro-Gly-Trp-Gly; |
| l. | Val-Pro-Ala-Tyr-Gly; |
| m. | Ala-Pro-Gly-Tyr-Gly; |
| n. | Ile-Pro-Gly-Tyr-Gly; |
| and | |
| o. | Leu-Pro-Gly-Tyr-Gly. |

151. The composition of claim 146, wherein the chemical functional group is selected from the group consisting of alkyne, halogens, alcohol, ketone, aldehyde, acyl halide, carbonate, carboxylate, carboxylic acid, ester, hydroperoxide, peroxide, ether, hemiacetal, hemiketal, acetal, ketal, acetal, orthoester, amide, amines, imine, imide, azide, azo compound, cyanates, nitrate, nitrile, nitrite, nitro compound, nitroso compound, pyridine, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, borinic ester, and a combination of two or more thereof.

152. The composition of claim 146, wherein the peptide-modifying molecule includes a polypeptide sequence comprising amino acids Pro, Ala, and Ser; a hydroxyethyl starch (HES) derivative; and a combination of two or more thereof.

153. The composition of claim 146, wherein the ligand is selected from a group consisting of a cell surface receptor ligand, a ligand, an antibody or a portion thereof, an antibody-like molecule, an enzyme, an antigen, a small molecule, a protein, a peptide, a peptidomimetic, a nucleic acid molecule, a carbohydrate, an aptamer, a cytokine, a lectin, a lipid, a plasma albumin, and a combination of two or more thereof.

154. The composition of claim 146, wherein the binding molecule is selected from the group consisting of biotin, avidin, streptavidin, immunoglobulin, protein A, protein G, hormone, receptor, receptor antagonist, receptor agonist, enzyme, enzyme cofactor, enzyme inhibitor, a charged molecule, carbohydrate, lectin, steroid, and a combination of two or more thereof.

155. The composition of claim 146, wherein the solid substrate is selected from the group consisting of a gold particle, a silver particle, a magnetic particle, a quantum dot, a fullerene, a carbon tube, a nanowire, a nanofibril, a grapheme, a polymer, collagen, albumin, silk, hyaluronic acid, and a combination of two or more thereof.

156. The composition of claim 146, further comprising an active agent distributed in the aggregate.

157. The composition of claim 146, wherein the aggregate is in a form of a particle, a fiber, a rod, a ring, a vesicle, a prism, a gel, a hollow particle, or a combination of two or more thereof.

158. The composition of claim 146, wherein the aggregate is porous.

159. The composition of claim 146, wherein the aggregate has a size of about 10 nm to about 500 μm.

160. The composition of claim 146, wherein the self-assembling peptides aggregate in an aqueous solvent.

161. The composition of claim 146, wherein the aggregate is stimuli-responsive.

162. A method of modulating release of an active agent comprising:
exposing a stimulus-responsive composition of claim 161 to at least one stimulus, wherein the composition comprises an active agent distributed in the aggregate of self-assembling peptides,
thereby releasing the active agent from the composition upon exposure of the composition to the stimulus.

163. The method of claim 162, wherein the exposure of the composition to the stimulus induces a change in size, pore size and/or porosity of the aggregate, a change in interaction between the aggregate and at least one component of the composition, or a combination of two or more thereof.

164. The method of claim 162, wherein said at least one stimulus is selected from the group consisting of a change in light intensity and/or wavelength, a change in pH, a change in temperature, a change in humidity, and a combination of two or more thereof.

165. A method of inducing gel formation comprising:
exposing a solution or suspension of protein or polymer molecules to at least one stimulus, wherein the protein or polymer molecules are each conjugated to at least one self-assembling peptide, wherein the self-assembling peptide consists essentially of:
an amino acid sequence of (Y₁-X₁-X₂-X₃-X₄-X₃-Y₂)ₙ; and
at least one entity conjugated to the amino acid sequence,
wherein:
X₁ is valine (Val) or a conservative substitution thereof;
X₂ is proline (Pro) or a conservative substitution thereof;
X₃ is glycine (Gly) or a conservative substitution thereof;
X₄ in each nth unit is independently an amino acid residue;
Y₁ and Y₂ are each independently a linker, wherein the linker is selected from a bond, one amino acid residue or a group of amino acid residues;
n is an integer from 1 to 10; and
the entity is selected from a group consisting of —H, —OH, a chemical functional group, a ligand, an active agent, a therapeutic agent, a binding molecule, a coupling molecule, a labeling agent, a peptide-modifying molecule, and a solid substrate;
thereby inducing aggregation of the self-assembling peptides to form a gel from the protein or polymer solution upon exposure to the stimulus.

166. The method of claim 165, wherein said at least one stimulus is selected from the group consisting of a change in light intensity and/or wavelength, a change in pH, a change in temperature, a change in humidity, and a combination of two or more thereof.
